SEQUENCE LISTING (11 sequences)

SEQ ID NO: 1 - DNA, 5940 bp, Mus musculus
atggcccaggcactgctggtacccccgggacctgagagcttccgccttttcactcgagaatctcttgctgctatcgaaaagcgtgctgcagaagagaaagccaagaaacccaagaaagaacaagacattgacgatgagaacaagccaaagccaaacagtgacttggaagctgggaagaaccttccatttatctatggagacattcctccagagatggtgtcggagcctctggaggacctggacccctactacgtcagtaagaaaacttttgtagtgttgaataaagggaaggcaatttttcgattcagtgccacctccgccttgtatattttaactccactaaaccccgttaggaaaattgctattaagattttggtacactctttattcagcatgcttatcatgtgcactattttgaccaactgtgtatttatgacattgagcaatcctcctgactggacgaagaatgtagagtacacattcactgggatctatacctttgagtcacttataaagatcttggccagaggattctgcttagaagatttcacatttcttcgtgacccatggaactggctggatttcagtgtcatcgtgatggcatatgtgacagagtttgtggacctgggcaatgtctcagcgctgagaacgttcagagttctccgagcattgaaaacaatatcagtcattccaggtttaaagaccatcgtgggggccctgatccagtcggtgaagaagctgtctgacgtcatgatactcactgtgttctgtctgagcgtctttgctctcatcgggctgcagctcttcatgggcaacctgaggaataaatgcttgcagtggcctccaagcgattctgcttttgagatcaacactacttcctacttcaatggcacaatggactcaaatgggacatttgttaatgtaacaatgagcacattcaactggaaggactatatcgcagatgacagccacttttatgttttggatggacaaaaagatcctttactttgtggaaatgggtccgatgcaggacaatgtccagaagggtacatctgtgtgaaggctggacgaaaccccaactacggttacacgagctttgacacatttagctgggccttcttatcgctgtttcgactcatgactcaagactactgggagaacctttaccagttgacattacgtgcagctgggaaaacctacatgatctttttcgtcctggtaattttcttgggctcattttatttggtgaacttgatcctggctgtggtggccatggcctatgaggaacaaaatcaggccacactggaggaggctgagcagaaagaggcggagtttcagcagatgttggagcagttgaaaaagcagcaagaggaggctcaggcggtggcagctgcctcagcagcgtccagagacttcagtgggataggagggttaggagaacttctggagagttcttcagaagcttccaagttgagctccaagagtgccaaggagtggaggaatcggaggaagaagaggagacagagggagcacttggagggaaaccacagacccgaaggagacaggtttcccaagtcggaatcagaagacagcgtcaagcgaaggagtttcctgttctccctggatgggaacccgctgagcggcgacaagaagctgtgctctccccatcagtctctcttgagtatccgtggctccctgttttccccaagacgcaatagcaaaacgagcattttcagcttcagaggtcgggcgaaggacgtggggtctgagaatgactttgcggatgatgaacacagcacctttgaagatagcgagagcaggagagactcactgtttgtgccgcacagacctggagagcgacgcaacagtaacgttagtcaggccagtatgtcatccaggatggtgccagggcttccagccaatgggaagatgcacagcactgtggattgcaatggtgtggtttccttgggtaccaccactgaaacagaagtcaggaagagaaggctaagttcttaccagatctcgatggaaatgctggaggattcctctgggagacaaagagccatgagcatagccagtatcctgaccaacacgatggaggaacttgaagaatctagacagaagtgtccaccatgctggtatagatttgccaatgtgtttttgatctgggactgttgtgattcatggttgaaagtaaagcatcttgtgaatttgattgtgatggatccatttgttgacctggccatcaccatctgcatcgtgttaaacacactgttcatggccatggagcactacccgatgacggagcagttcagcagtgtgctgacggtgggaaacctggtcttcaccgggatcttcacagccgagatggtcctgaaaatcatcgcaatggatccctattactatttccaagagggctggaatatctttgatggaattattgttagcctgagtttaatggagcttggcctggcaaacgtggaggggctgtccgtgcttcggtccttcagactgctgcgagtcttcaagttggcaaaatcctggcccacactgaatatgctcattaagatcatcggcaactcggtgggcgcactgggcaacctgaccctggtgctggccatcatcgtcttcatttttgccgtggtcggcatgcagctgtttggaaagagctacaaggagtgtgtttgcaagatcaatgaggactgcaagctcccgcgctggcacatgaacgacttcttccactccttcctgatagtgttccgcgtgctgtgtggggagtggatagagaccatgtgggactgcatggaggtcgcgggccagaccatgtgccttattgtgtttatgttggtcatggtgattgggaaccttgtggttctgaacctcttcctggccttattgttgagttcctttagttcagacaaccttgctgctacggacgatgataacgaaatgaacaacctccagatcgcggtgggaaggatgcaaaaggggattgattatgtgaaaaataagatacgggagtgcttccgaaaagcgttttttagaaagccgaaagtgatagaaatccacgaagggaacaaaatagacagctgcatgtccaataacacgggcgtagttgaaataagcaaagagcttaactaccttaaagacggtaacggaaccaccagtggcgtgggtactggaagcagtgtggagaaatacgtaattgatgaaaatgactacatgtcattcatcaacaaccccagcctcaccgtgacggtgccaattgccgtgggagagtctgactttgaaaatttaaacacggaagagtttagcagtgagtcagaactggaagaaagcaaggagaaattaaatgcaaccagctcttctgaaggaagcacagttgacgttgctccgccccgagaaggtgaacaagctgaaattgaacctgaggaggaccttaagccagaagcttgctttactgaaggatgcattaaaaaatttcccttctgccaagtaagtacggaagaaggtaaaggaaaaatctggtggaatcttaggaagacctgctatagcattgtggaacacaactggtttgagacgttcattgtgttcatgattctcctcagtagtggtgctttggcctttgaagatatatacattgagcaacggaagaccatcaaaaccatgctggagtatgctgacaaagtcttcacttacatcttcatcctggaaatgctcctcaaatgggtggcctatggatttcaaacctatttcaccaatgcctggtgctggttggacttcttgattgttgatgtttctttggttagcctggtggccaacgctcttggctattcagaacttggtgccatcaaatccctacggaccctgagagctctgaggccgctccgagccttatcccgctttgaaggcatgagggtggttgtgaacgctcttgttggtgcaatcccctccatcatgaatgtgctactggtgtgcctcatcttctggttaatctttagtatcatgggtgtgaatctgtttgctggaaagttctatcactgtgttaacatgacaacgggcagcatgttcgacatgagtgaagtcaacaatttcagcgactgtcaggctcttggcaagcaagcccgatggaagaatgtgaaagtcaactttgacaatgttggggctggctacctggcattgctgcaagtggccacattcaaaggctggatggatattatgtatgcagctgtggattcacgggacgtcaaactgcagcctgtatatgaagaaaatctgtacatgtatctgtactttgtcatcttcatcatctttgggtcgttcttcactctaaatctattcatcggcgtcatcatagacaacttcaaccagcagaagaagaagtttggaggtcaagacatctttatgacagaagagcagaaaaagtactacaatgcaatgaagaaacttggctccaaaaaacctcagaagcccatccctcgacctgcaaacaaatttcaaggaatggtctttgactttgtaaccagacaagtgtttgacatcagcatcatgatcctcatctgcctcaacatggtgaccatgatggtggaaacggacgaccagagcaaatacatgaccctggttttgtcccgaatcaacctggtattcatcgtcctcttcactggggagtttctgctgaagctcatctctctcagatactactacttcacgattggctggaacatctttgactttgtggtggtgattctctcaattgtaggaatgttccttgctgagctgatagagaagtattttgtgtctcctaccctgttccgagtcatccgcctggccaggattggacgaatcctacgcctgatcaaaggcgccaaggggatccgcacgctgctctttgctctgatgatgtcccttcctgcgctgttcaacatcggcctcctgcttttcctcgtcatgttcatctacgccatctttgggatgtccaactttgcctatgttaaaaaagaggctggaattgatgacatgttcaactttgagacttttggcaacagcatgatctgcctgttccaaatcaccacctctgcgggctgggatggactgttggcccccatcctcaacagtgcacctcctgactgtgaccctgatgcaattcaccctggaagctcagtgaagggagactgtgggaacccatctgtggggattttcttttttgtcagctacatcatcatatccttcctggttgtggtgaacatgtacattgctgtcatcctggagaacttcagcgttgccacagaagaaagtgcagagcccctgagtgaggacgactttgagatgttctacgaggtctgggagaagttcgaccctgacgccacccagttcatagagttctgcaagctctctgactttgcagctgccctggatcctcccctcctcatcgcaaagccaaacaaagtccagctcattgccatggacctgcccatggtgagtggagaccgcatccactgcctggacatcttatttgcttttacaaagcgggtcctgggtgagagtggagagatggatgcccttcgaatccagatggaagatcggttcatggcttccaatccctccaaggtctcttatgagcccattaccaccactctgaagcgcaaacaagaggaggtgtctgctgctatcattcagcgtaattacagatgttatcttttaaagcaaaggttaaaaaacatatcaaatacgtatgacaaagagacaatcaaggggaggattgtcttgcctataaaaggagatatggttattgacaaattaaatgggaattccaccccagaaaagacagatgggagttcctctaccacctcccctccttcctatgacagtgtaacaaaaccagataaggaaaagtttgagaaagacaaaccagaaaaagaaagcaaagggaaagaggtctga

SEQ ID NO: 2 - protein, 1979 aa, Mus musculus
MAQALLVPPGPESFRLFTRESLAAIEKRAAEEKAKKPKKEQDIDDENKPKPNSDLEAGKNLPFIYGDIPPEMVSEPLEDLDPYYVSKKTFVVLNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVDLGNVSALRTFRVLRALKTISVIPGLKTIVGALIQSVKKLSDVMILTVFCLSVFALIGLQLFMGNLRNKCLQWPPSDSAFEINTTSYFNGTMDSNGTFVNVTMSTFNWKDYIADDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGIGGLGELLESSSEASKLSSKSAKEWRNRRKKRRQREHLEGNHRPEGDRFPKSESEDSVKRRSFLFSLDGNPLSGDKKLCSPHQSLLSIRGSLFSPRRNSKTSIFSFRGRAKDVGSENDFADDEHSTFEDSESRRDSLFVPHRPGERRNSNVSQASMSSRMVPGLPANGKMHSTVDCNGVVSLGTTTETEVRKRRLSSYQISMEMLEDSSGRQRAMSIASILTNTMEELEESRQKCPPCWYRFANVFLIWDCCDSWLKVKHLVNLIVMDPFVDLAITICIVLNTLFMAMEHYPMTEQFSSVLTVGNLVFTGIFTAEMVLKIIAMDPYYYFQEGWNIFDGIIVSLSLMELGLANVEGLSVLRSFRLLRVFKLAKSWPTLNMLIKIIGNSVGALGNLTLVLAIIVFIFAVVGMQLFGKSYKECVCKINEDCKLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKIRECFRKAFFRKPKVIEIHEGNKIDSCMSNNTGVVEISKELNYLKDGNGTTSGVGTGSSVEKYVIDENDYMSFINNPSLTVTVPIAVGESDFENLNTEEFSSESELEESKEKLNATSSSEGSTVDVAPPREGEQAEIEPEEDLKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNMTTGSMFDMSEVNNFSDCQALGKQARWKNVKVNFDNVGAGYLALLQVATFKGWMDIMYAAVDSRDVKLQPVYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQSKYMTLVLSRINLVFIVLFTGEFLLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDAIHPGSSVKGDCGNPSVGIFFFVSYIIISFLVVVNMYIAVILENFSVATEESAEPLSEDDFEMFYEVWEKFDPDATQFIEFCKLSDFAAALDPPLLIAKPNKVQLIAMDLPMVSGDRIHCLDILFAFTKRVLGESGEMDALRIQMEDRFMASNPSKVSYEPITTTLKRKQEEVSAAIIQRNYRCYLLKQRLKNISNTYDKETIKGRIVLPIKGDMVIDKLNGNSTPEKTDGSSSTTSPPSYDSVTKPDKEKFEKDKPEKESKGKEV

SEQ ID NO: 3 - DNA, 6071 bp, Mus musculus
tgaaaagatggcccaggcactgctggtacccccgggacctgagagcttccgccttttcactcgagaatctcttgctgctatcgaaaagcgtgctgcagaagagaaagccaagaaacccaagaaagaacaagacattgacgatgagaacaagccaaagccaaacagtgacttggaagctgggaagaaccttccatttatctatggagacattcctccagagatggtgtcggagcctctggaggacctggacccctactacgtcagtaagaaaacttttgtagtgttgaataaagggaaggcaatttttcgattcagtgccacctccgccttgtatattttaactccactaaaccccgttaggaaaattgctattaagattttggtacactctttattcagcatgcttatcatgtgcactattttgaccaactgtgtatttatgacattgagcaatcctcctgactggacgaagaatgtagagtacacattcactgggatctatacctttgagtcacttataaagatcttggccagaggattctgcttagaagatttcacatttcttcgtgacccatggaactggctggatttcagtgtcatcgtgatggcatatgtgacagagtttgtggacctgggcaatgtctcagcgctgagaacgttcagagttctccgagcattgaaaacaatatcagtcattccaggtttaaagaccatcgtgggggccctgatccagtcggtgaagaagctgtctgacgtcatgatactcactgtgttctgtctgagcgtctttgctctcatcgggctgcagctcttcatgggcaacctgaggaataaatgcttgcagtggcctccaagcgattctgcttttgagatcaacactacttcctacttcaatggcacaatggactcaaatgggacatttgttaatgtaacaatgagcacattcaactggaaggactatatcgcagatgacagccacttttatgttttggatggacaaaaagatcctttactttgtggaaatgggtccgatgcaggacaatgtccagaagggtacatctgtgtgaaggctggacgaaaccccaactacggttacacgagctttgacacatttagctgggccttcttatcgctgtttcgactcatgactcaagactactgggagaacctttaccagttgacattacgtgcagctgggaaaacctacatgatctttttcgtcctggtaattttcttgggctcattttatttggtgaacttgatcctggctgtggtggccatggcctatgaggaacaaaatcaggccacactggaggaggctgagcagaaagaggcggagtttcagcagatgttggagcagttgaaaaagcagcaagaggaggctcaggcggtggcagctgcctcagcagcgtccagagacttcagtgggataggagggttaggagaacttctggagagttcttcagaagcttccaagttgagctccaagagtgccaaggagtggaggaatcggaggaagaagaggagacagagggagcacttggagggaaaccacagacccgaaggagacaggtttcccaagtcggaatcagaagacagcgtcaagcgaaggagtttcctgttctccctggatgggaacccgctgagcggcgacaagaagctgtgctctccccatcagtctctcttgagtatccgtggctccctgttttccccaagacgcaatagcaaaacgagcattttcagcttcagaggtcgggcgaaggacgtggggtctgagaatgactttgcggatgatgaacacagcacctttgaagatagcgagagcaggagagactcactgtttgtgccgcacagacctggagagcgacgcaacagtaacgttagtcaggccagtatgtcatccaggatggtgccagggcttccagccaatgggaagatgcacagcactgtggattgcaatggtgtggtttccttgggtaccaccactgaaacagaagtcaggaagagaaggctaagttcttaccagatctcgatggaaatgctggaggattcctctgggagacaaagagccatgagcatagccagtatcctgaccaacacgatggaggaacttgaagaatctagacagaagtgtccaccatgctggtatagatttgccaatgtgtttttgatctgggactgttgtgattcatggttgaaagtaaagcatcttgtgaatttgattgtgatggatccatttgttgacctggccatcaccatctgcatcgtgttaaacacactgttcatggccatggagcactacccgatgacggagcagttcagcagtgtgctgacggtgggaaacctggtcttcaccgggatcttcacagccgagatggtcctgaaaatcatcgcaatggatccctattactatttccaagagggctggaatatctttgatggaattattgttagcctgagtttaatggagcttggcctggcaaacgtggaggggctgtccgtgcttcggtccttcagactgctgcgagtcttcaagttggcaaaatcctggcccacactgaatatgctcattaagatcatcggcaactcggtgggcgcactgggcaacctgaccctggtgctggccatcatcgtcttcatttttgccgtggtcggcatgcagctgtttggaaagagctacaaggagtgtgtttgcaagatcaatgaggactgcaagctcccgcgctggcacatgaacgacttcttccactccttcctgatagtgttccgcgtgctgtgtggggagtggatagagaccatgtgggactgcatggaggtcgcgggccagaccatgtgccttattgtgtttatgttggtcatggtgattgggaaccttgtggttctgaacctcttcctggccttattgttgagttcctttagttcagacaaccttgctgctacggacgatgataacgaaatgaacaacctccagatcgcggtgggaaggatgcaaaaggggattgattatgtgaaaaataagatacgggagtgcttccgaaaagcgttttttagaaagccgaaagtgatagaaatccacgaagggaacaaaatagacagctgcatgtccaataacacgggcgtagttgaaataagcaaagagcttaactaccttaaagacggtaacggaaccaccagtggcgtgggtactggaagcagtgtggagaaatacgtaattgatgaaaatgactacatgtcattcatcaacaaccccagcctcaccgtgacggtgccaattgccgtgggagagtctgactttgaaaatttaaacacggaagagtttagcagtgagtcagaactggaagaaagcaaggagaaattaaatgcaaccagctcttctgaaggaagcacagttgacgttgctccgccccgagaaggtgaacaagctgaaattgaacctgaggaggaccttaagccagaagcttgctttactgaaggatgcattaaaaaatttcccttctgccaagtaagtacggaagaaggtaaaggaaaaatctggtggaatcttaggaagacctgctatagcattgtggaacacaactggtttgagacgttcattgtgttcatgattctcctcagtagtggtgctttggcctttgaagatatatacattgagcaacggaagaccatcaaaaccatgctggagtatgctgacaaagtcttcacttacatcttcatcctggaaatgctcctcaaatgggtggcctatggatttcaaacctatttcaccaatgcctggtgctggttggacttcttgattgttgatgtttctttggttagcctggtggccaacgctcttggctattcagaacttggtgccatcaaatccctacggaccctgagagctctgaggccgctccgagccttatcccgctttgaaggcatgagggtggttgtgaacgctcttgttggtgcaatcccctccatcatgaatgtgctactggtgtgcctcatcttctggttaatctttagtatcatgggtgtgaatctgtttgctggaaagttctatcactgtgttaacatgacaacgggcagcatgttcgacatgagtgaagtcaacaatttcagcgactgtcaggctcttggcaagcaagcccgatggaagaatgtgaaagtcaactttgacaatgttggggctggctacctggcattgctgcaagtggccacattcaaaggctggatggatattatgtatgcagctgtggattcacgggacgtcaaactgcagcctgtatatgaagaaaatctgtacatgtatctgtactttgtcatcttcatcatctttgggtcgttcttcactctaaatctattcatcggcgtcatcatagacaacttcaaccagcagaagaagaagtttggaggtcaagacatctttatgacagaagagcagaaaaagtactacaatgcaatgaagaaacttggctccaaaaaacctcagaagcccatccctcgacctgcaaacaaatttcaaggaatggtctttgactttgtaaccagacaagtgtttgacatcagcatcatgatcctcatctgcctcaacatggtgaccatgatggtggaaacggacgaccagagcaaatacatgaccctggttttgtcccgaatcaacctggtattcatcgtcctcttcactggggagtttctgctgaagctcatctctctcagatactactacttcacgattggctggaacatctttgactttgtggtggtgattctctcaattgtaggaatgttccttgctgagctgatagagaagtattttgtgtctcctaccctgttccgagtcatccgcctggccaggattggacgaatcctacgcctgatcaaaggcgccaaggggatccgcacgctgctctttgctctgatgatgtcccttcctgcgctgttcaacatcggcctcctgcttttcctcgtcatgttcatctacgccatctttgggatgtccaactttgcctatgttaaaaaagaggctggaattgatgacatgttcaactttgagacttttggcaacagcatgatctgcctgttccaaatcaccacctctgcgggctgggatggactgttggcccccatcctcaacagtgcacctcctgactgtgaccctgatgcaattcaccctggaagctcagtgaagggagactgtgggaacccatctgtggggattttcttttttgtcagctacatcatcatatccttcctggttgtggtgaacatgtacattgctgtcatcctggagaacttcagcgttgccacagaagaaagtgcagagcccctgagtgaggacgactttgagatgttctacgaggtctgggagaagttcgaccctgacgccacccagttcatagagttctgcaagctctctgactttgcagctgccctggatcctcccctcctcatcgcaaagccaaacaaagtccagctcattgccatggacctgcccatggtgagtggagaccgcatccactgcctggacatcttatttgcttttacaaagcgggtcctgggtgagagtggagagatggatgcccttcgaatccagatggaagatcggttcatggcttccaatccctccaaggtctcttatgagcccattaccaccactctgaagcgcaaacaagaggaggtgtctgctgctatcattcagcgtaattacagatgttatcttttaaagcaaaggttaaaaaacatatcaaatacgtatgacaaagagacaatcaaggggaggattgtcttgcctataaaaggagatatggttattgacaaattaaatgggaattccaccccagaaaagacagatgggagttcctctaccacctcccctccttcctatgacagtgtaacaaaaccagataaggaaaagtttgagaaagacaaaccagaaaaagaaagcaaagggaaagaggtctgagagaatcaaaagtaaaaaaacaaaacaaaaaaaatttcaaaaatttaaaaaggaaacaaagaaatgtctttgtaatcaattgtttacagcctctgaaggtaaagtgtccgtgtcaactggactc

SEQ ID NO: 4 - DNA, 9123 bp, Homo sapiens
agcgaagcggaggcataagcagagaggattctggaaaggtctctttgttttcttatccacagagaaagaaagaaaaaaaattgtaactaatttgtaaacctctgtggtcaaaaaaaaaaaaaaaaaaaaagctgaacagctgccagaggaagacacgttataccctaaccatcttggatgctgggctttgttatgctgtaattcataaggctctgttttatcagagattatggagcaagaaaactgaagccaagccacatcaaggtttgacagggatgagatacctgtcaaggattcatagtagagtggcttactgggaaaggagcaaagaatctcttctagggatattgtaagaataaatgagataattcacagaagggacctggagcttttccggaaaaaggtgctgtgactatctaaggtaattcgtatgcaagaagctacacgtaattaaatgtgcaggatgaaaagatggcacaggcactgttggtacccccaggacctgaaagcttccgcctttttactagagaatctcttgctgctatcgaaaaacgtgctgcagaagagaaagccaagaagcccaaaaaggaacaagataatgatgatgagaacaaaccaaagccaaatagtgacttggaagctggaaagaaccttccatttatttatggagacattcctccagagatggtgtcagagcccctggaggacctggatccctactatatcaataagaaaacttttatagtaatgaataaaggaaaggcaattttccgattcagtgccacctctgccttgtatattttaactccactaaaccctgttaggaaaattgctatcaagattttggtacattctttattcagcatgcttatcatgtgcactattttgaccaactgtgtatttatgaccttgagcaaccctcctgactggacaaagaatgtagagtacacattcactggaatctatacctttgagtcacttataaaaatcttggcaagagggttttgcttagaagattttacgtttcttcgtgatccatggaactggctggatttcagtgtcattgtgatggcatatgtgacagagtttgtggacctgggcaatgtctcagcgttgagaacattcagagttctccgagcactgaaaacaatttcagtcattccaggtttaaagaccattgtgggggccctgatccagtcggtaaagaagctttctgatgtgatgatcctgactgtgttctgtctgagcgtgtttgctctcattgggctgcagctgttcatgggcaatctgaggaataaatgtttgcagtggcccccaagcgattctgcttttgaaaccaacaccacttcctactttaatggcacaatggattcaaatgggacatttgttaatgtaacaatgagcacatttaactggaaggattacattggagatgacagtcacttttatgttttggatgggcaaaaagaccctttactctgtggaaatggctcagatgcaggccagtgtccagaaggatacatctgtgtgaaggctggtcgaaaccccaactatggctacacaagctttgacacctttagctgggctttcctgtctctatttcgactcatgactcaagactattgggaaaatctttaccagttgacattacgtgctgctgggaaaacatacatgatattttttgtcctggtcattttcttgggctcattttatttggtgaatttgatcctggctgtggtggccatggcctatgaggagcagaatcaggccaccttggaagaagcagaacaaaaagaggccgaatttcagcagatgctcgaacagcttaaaaagcaacaggaagaagctcaggcagttgcggcagcatcagctgcttcaagagatttcagtggagtaggtgggttaggagagctgttggaaagttcttcagaagcatcaaagttgagttccaaaggtgctaaagaatggaggaaccggaggaagaaaagaagacagagagagcaccttgaaggaaacaacaaaggagagagagacagctttcccaaatccgaatctgaagacagcgtcaaaagaagcagcttccttttctccatggatggaaacagactgaccagtgacaaaaaattctgctcccctcatcagtctctcttgagtatccgtggctccctgttttccccaagacgcaatagcaaaacaagcattttcagtttcagaggtcgggcaaaggatgttggatctgaaaatgactttgctgatgatgaacacagcacatttgaagacggcgaaagcaggagagactcactgtttgtgccgcacagacatggagagcgacgcaacagtaacgttagtcaggccagtatgtcatccaggatggtgccagggcttccagcaaatgggaagatgcacagcactgtggattgcaatggtgtggtttccttggtgggtggaccttcagctctaacgtcacctactggacaacttcccccagagggcaccaccactgaaacggaagtcagaaagagaaggttaagctcttaccagatttcaatggagatgctggaggattcctctggaaggcaaagagccgtgagcatagccagcattctgaccaacacaatggaagaacttgaagaatctagacagaaatgtccgccatgctggtatagatttgccaatgtgttcttgatctgggactgctgtgatgcatggttaaaagtaaaacatcttgtgaatttaattgttatggatccatttgttgatcttgccatcactatttgcattgtcttaaataccctctttatggccatggagcactaccccatgactgagcaattcagtagtgtgttgactgtaggaaacctggtctttactgggattttcacagcagaaatggttctcaagatcattgccatggatccttattactatttccaagaaggctggaatatctttgatggaattattgtcagcctcagtttaatggagcttggtctgtcaaatgtggagggattgtctgtactgcgatcattcagactgcttagagttttcaagttggcaaaatcctggcccacactaaatatgctaattaagatcattggcaattctgtgggggctctaggaaacctcaccttggtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggccaaaccatgtgccttattgttttcatgttggtcatggtcattggaaaccttgtggttctgaacctctttctggccttattattgagttcatttagctcagacaaccttgctgctactgatgatgacaatgaaatgaataatctgcagattgcagtaggaagaatgcaaaagggaattgattatgtgaaaaataagatgcgggagtgtttccaaaaagccttttttagaaagccaaaagttatagaaatccatgaaggcaataagatagacagctgcatgtccaataatactggaattgaaataagcaaagagcttaattatcttagagatgggaatggaaccaccagtggtgtaggtactggaagcagtgttgaaaaatacgtaatcgatgaaaatgattatatgtcattcataaacaaccccagcctcaccgtcacagtgccaattgctgttggagagtctgactttgaaaacttaaatactgaagagttcagcagtgagtcagaactagaagaaagcaaagagaaattaaatgcaaccagctcatctgaaggaagcacagttgatgttgttctaccccgagaaggtgaacaagctgaaactgaacccgaagaagactttaaaccggaagcttgttttactgaagggtgtattaaaaagtttccattctgtcaagtaagtacagaagaaggcaaagggaagatctggtggaatcttcgaaaaacctgctacagtattgttgagcacaactggtttgagactttcattgtgttcatgatccttctcagtagtggtgcattggcctttgaagatatatacattgaacagcgaaagactatcaaaaccatgctagaatatgctgacaaagtctttacctatatattcattctggaaatgcttctcaaatgggttgcttatggatttcaaacatatttcactaatgcctggtgctggctagatttcttgatcgttgatgtttctttggttagcctggtagccaatgctcttggctactcagaactcggtgccatcaaatcattacggacattaagagctttaagacctctaagagccttatcccggtttgaaggcatgagggtggttgtgaatgctcttgttggagcaattccctctatcatgaatgtgctgttggtctgtctcatcttctggttgatctttagcatcatgggtgtgaatttgtttgctggcaagttctaccactgtgttaacatgacaacgggtaacatgtttgacattagtgatgttaacaatttgagtgactgtcaggctcttggcaagcaagctcggtggaaaaacgtgaaagtaaactttgataatgttggcgctggctatcttgcactgcttcaagtggccacatttaaaggctggatggatattatgtatgcagctgttgattcacgagatgttaaacttcagcctgtatatgaagaaaatctgtacatgtatttatactttgtcatctttatcatctttgggtcattcttcactctgaatctattcattggtgtcatcatagataacttcaaccagcagaaaaagaagtttggaggtcaagacatctttatgacagaggaacagaaaaaatattacaatgcaatgaagaaacttggatccaagaaacctcagaaacccatacctcgcccagcaaacaaattccaaggaatggtctttgattttgtaaccagacaagtctttgatatcagcatcatgatcctcatctgcctcaacatggtcaccatgatggtggaaacggatgaccagggcaaatacatgaccctagttttgtcccggatcaacctagtgttcattgttctgttcactggagaatttgtgctgaagctcgtttccctcagacactactacttcactataggctggaacatctttgactttgtggtggtgattctctccattgtaggtatgtttctggctgagatgatagaaaagtattctgtgtcccctaccttgttccgagtgatccgtcttgccaggattggccgaatcctacgtctgatcaaaggagcaaaggggatccgcacgctgctctttgctttgatgatgtcccttcctgcgttgtttaacatcggcctcctgctcttcctggtcatgtttatctatgccatctttgggatgtccaactttgcctatgttaaaaaggaagctggaattgatgacatgttcaactttgagacctttggcaacagcatgatctgcttgttccaaattacaacctctgctggctgggatggattgctagcacctattcttaatagtgcaccacccgactgtgaccctgacacaattcaccctggcagctcagttaagggagaccgtggggacccatctgttgggattttcttttttgtcagttacatcatcatatccttcctggttgtggtgaacatgtacatcgcggtcatcctggagaacttcagtgttgctactgaagaaagtgcagagcccctgagtgaggatgactttgagatgttctatgaggtttgggaaaagtttgatcccgatgcgacccagtttatagagttctctaaactctctgattttgcagctgccctggatcctcctcttctcatagcaaaacccaacaaagtccagcttattgccatggatctgcccatggtcagtggtgaccggatccactgtcttgatattttatttgcctttacaaagcgtgttttgtgtgagagtggagagatggatgcccttcgaatacagatggaagacaggtttatggcatcaaacccctccaaagtctcttatgagcctattacaaccactttgaaacgtaaacaagaggaggtgtctgccgctatcattcagcgtaatttcagatgttatcttttaaagcaaaggttaaaaaatatatcaagtaactataacaaagaggcaattaaagggaggattgacttacctataaaacaagacatgattattgacaaactaaatgggaactccactccagaaaaaacagatgggagttcctctaccacccctcctccttcctatgatagtgtaacaaaaccagacaaggaaaagtttgagaaagacaaaccagaaaaagaaagcaaaggaaaagaggtcagagaaaatcaaaagtaaaaagaaacaaagaattatctttgtgatcaattgtttacagcctatgaaggtaaagtatatgtgtcaactggacttcaagaggaggtccatgccaaactgactgttttaacaaatactcatagtcagtgcctatacaagacagtgaagtgacctctctgtcactgcaactctgtgaagcagggtatcaacgttgacaagaggttgctgtttttattaccagctgacactgctgaggagaaacccaatggctacctagactatagggatagttgtgcaaagtgaacattgtaactacaccaaacacctttagtacagtccttgcatccattctatttttaacttccatatctgccatatttttacaaaatttgttctagtgcatttccatggtccccaattcatagtttattcataatgctatgtcactatttttgtaaatgaggtttacgttgaagaaacagtatacaagaaccctgtctctcaaatgatcagacaaaggtgttttgccagagagataaaatttttgctcaaaaccagaaaaagaattgtaatggctacagtttcagttacttccattttctagatggctttaattttgaaagtattttagtctgttatgtttgtttctatctgaacagttatgtgcctgtaaagtctcctctaatatttaaaggattatttttatgcaaagtattctgtttcagcaagtgcaaattttattctaagtttcagagctctatatttaatttaggtcaaatgctttccaaaaagtaatctaataaatccattctagaaaaatatatctaaagtattgctttagaatagttgttccactttctgctgcagtattgctttgccatcttctgctctcagcaaagctgatagtctatgtcaattaaataccctatgttatgtaaatagttattttatcctgtggtgcatgtttgggcaaatatatatatagcctgataaacaacttctattaaatcaaatatgtaccacagtgtatgtgtcttttgcaagcttccaacagggatgtatcctgtatcattcattaaacatagtttaaaggctatcactaatgcatgttaatattgcctatgctgctctattttactcaatccattcttcacaagtcttggttaaagaatgtcacatattggtgatagaatgaattcaacctgctctgtccattatgtcaagcagaataatttgaagctatttacaaacacctttacttttgcacttttaattcaacatgagtatcatatggtatctctctggatttcaaggaaacacactggatactgcctactgacaaaacctattcttcatattttgctaaaaatatgtctaaaacttgtttaaatataaataatgtaaaaatataatcaactttatttgtcagcattttgtacataagaaaattattttcaggttgatgacatcacaatttattttactttatgcttttgcttttgatttttaatcacaattccaaacttttgaatccataagatttttcaatggataatttcctaaaataaaagttagataatgggttttatggatttctttgttataatatattttctaccattccaataggagatacattggtcaaacactcaaacctagatcattttctaccaactatggttgcctcaatataaccttttattcatagatgtttttttttattcaacttttgtagtatttacgtatgcagactagtcttatttttttaattcctgctgcactaaagctattacaaatataacatggactttgttctttttagccatgaacaaagtggcaaagttgtgcaattacctaacatgatataaatttttgttttttgcacaaaccaaaagtttaatgttaattctttttacaaaactatttactgtagtgtattgaagaactgcatgcagggaattgctattgctaaaaagaatggtgagctacgtcattattgagccaaaagaataaatttcattttttattgcatttcacttattgggctctggggttttttgtttttgttttttgctgttggcagtttaaaatatatataattaataaaacctgtgcttgatctgacatttgtatacataaaagtttacatgaattttacaacaaactagtgcatgattcaccaagcagtactacagaacaaaggcaaattaaaagcagctttgtgaacttttatgtgtgcaaaggatcaagttcacatgttccaactttcaggtttgataataatagtagtaaccacctacaatagctttcaatttcaattaactcccttggctataagcatctaaactcatcttctttcaatataattgatgctatctcctaattacttggtggctaataaatgttacattctttgttacttaaatgcattatataaactcctatgtatacataaggtattaatgatatagttattgagaatttatattaacttttttttcaagaacccttggatttatgtgaggtcaaaaccaaactcttattctcagtggaaaactccagttgtaatgcatatttttaaagacaatttggatctaaatatgtatttcataattctcccataataaattatataaggtggaaaaaaaaaaaaaaaaaaaaaaaaaaaaa

SEQ ID NO: 5 - protein, 2000 aa, Homo sapiens
MAQALLVPPGPESFRLFTRESLAAIEKRAAEEKAKKPKKEQDNDDENKPKPNSDLEAGKNLPFIYGDIPPEMVSEPLEDLDPYYINKKTFIVMNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVDLGNVSALRTFRVLRALKTISVIPGLKTIVGALIQSVKKLSDVMILTVFCLSVFALIGLQLFMGNLRNKCLQWPPSDSAFETNTTSYFNGTMDSNGTFVNVTMSTFNWKDYIGDDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGVGGLGELLESSSEASKLSSKGAKEWRNRRKKRRQREHLEGNNKGERDSFPKSESEDSVKRSSFLFSMDGNRLTSDKKFCSPHQSLLSIRGSLFSPRRNSKTSIFSFRGRAKDVGSENDFADDEHSTFEDGESRRDSLFVPHRHGERRNSNVSQASMSSRMVPGLPANGKMHSTVDCNGVVSLVGGPSALTSPTGQLPPEGTTTETEVRKRRLSSYQISMEMLEDSSGRQRAVSIASILTNTMEELEESRQKCPPCWYRFANVFLIWDCCDAWLKVKHLVNLIVMDPFVDLAITICIVLNTLFMAMEHYPMTEQFSSVLTVGNLVFTGIFTAEMVLKIIAMDPYYYFQEGWNIFDGIIVSLSLMELGLSNVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKMRECFQKAFFRKPKVIEIHEGNKIDSCMSNNTGIEISKELNYLRDGNGTTSGVGTGSSVEKYVIDENDYMSFINNPSLTVTVPIAVGESDFENLNTEEFSSESELEESKEKLNATSSSEGSTVDVVLPREGEQAETEPEEDFKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNMTTGNMFDISDVNNLSDCQALGKQARWKNVKVNFDNVGAGYLALLQVATFKGWMDIMYAAVDSRDVKLQPVYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQGKYMTLVLSRINLVFIVLFTGEFVLKLVSLRHYYFTIGWNIFDFVVVILSIVGMFLAEMIEKYSVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDTIHPGSSVKGDRGDPSVGIFFFVSYIIISFLVVVNMYIAVILENFSVATEESAEPLSEDDFEMFYEVWEKFDPDATQFIEFSKLSDFAAALDPPLLIAKPNKVQLIAMDLPMVSGDRIHCLDILFAFTKRVLCESGEMDALRIQMEDRFMASNPSKVSYEPITTTLKRKQEEVSAAIIQRNFRCYLLKQRLKNISSNYNKEAIKGRIDLPIKQDMIIDKLNGNSTPEKTDGSSSTTPPPSYDSVTKPDKEKFEKDKPEKESKGKEVRENQK

SEQ ID NO: 6 - DNA, 8976 bp, Homo sapiens
agcgaagcggaggcataagcagagaggattctggaaaggtctctttgttttcttatccacagagaaagaaagaaaaaaaattgtaactaatttgtaaacctctgtggtcaaaaaaaaaaaaaaaaaaaaagctgaacagctgccagaggaagacacgttataccctaaccatcttggatgctgggctttgttatgctgtaattcataaggctctgttttatcagagattatggagcaagaaaactgaagccaagccacatcaaggtttgacagggatgagatacctgtcaaggattcatagtagagtggcttactgggaaaggagcaaagaatctcttctagggatattgtaagaataaatgagataattcacagaagggacctggagcttttccggaaaaaggtgctgtgactatctaaggtaattcgtatgcaagaagctacacgtaattaaatgtgcaggatgaaaagatggcacaggcactgttggtacccccaggacctgaaagcttccgcctttttactagagaatctcttgctgctatcgaaaaacgtgctgcagaagagaaagccaagaagcccaaaaaggaacaagataatgatgatgagaacaaaccaaagccaaatagtgacttggaagctggaaagaaccttccatttatttatggagacattcctccagagatggtgtcagagcccctggaggacctggatccctactatatcaataagaaaacttttatagtaatgaataaaggaaaggcaattttccgattcagtgccacctctgccttgtatattttaactccactaaaccctgttaggaaaattgctatcaagattttggtacattctttattcagcatgcttatcatgtgcactattttgaccaactgtgtatttatgaccttgagcaaccctcctgactggacaaagaatgtagagtacacattcactggaatctatacctttgagtcacttataaaaatcttggcaagagggttttgcttagaagattttacgtttcttcgtgatccatggaactggctggatttcagtgtcattgtgatggcatatgtgacagagtttgtggacctgggcaatgtctcagcgttgagaacattcagagttctccgagcactgaaaacaatttcagtcattccaggtttaaagaccattgtgggggccctgatccagtcggtaaagaagctttctgatgtgatgatcctgactgtgttctgtctgagcgtgtttgctctcattgggctgcagctgttcatgggcaatctgaggaataaatgtttgcagtggcccccaagcgattctgcttttgaaaccaacaccacttcctactttaatggcacaatggattcaaatgggacatttgttaatgtaacaatgagcacatttaactggaaggattacattggagatgacagtcacttttatgttttggatgggcaaaaagaccctttactctgtggaaatggctcagatgcaggccagtgtccagaaggatacatctgtgtgaaggctggtcgaaaccccaactatggctacacaagctttgacacctttagctgggctttcctgtctctatttcgactcatgactcaagactattgggaaaatctttaccagttgacattacgtgctgctgggaaaacatacatgatattttttgtcctggtcattttcttgggctcattttatttggtgaatttgatcctggctgtggtggccatggcctatgaggagcagaatcaggccaccttggaagaagcagaacaaaaagaggccgaatttcagcagatgctcgaacagcttaaaaagcaacaggaagaagctcaggcagttgcggcagcatcagctgcttcaagagatttcagtggagtaggtgggttaggagagctgttggaaagttcttcagaagcatcaaagttgagttccaaaggtgctaaagaatggaggaaccggaggaagaaaagaagacagagagagcaccttgaaggaaacaacaaaggagagagagacagctttcccaaatccgaatctgaagacagcgtcaaaagaagcagcttccttttctccatggatggaaacagactgaccagtgacaaaaaattctgctcccctcatcagtctctcttgagtatccgtggctccctgttttccccaagacgcaatagcaaaacaagcattttcagtttcagaggtcgggcaaaggatgttggatctgaaaatgactttgctgatgatgaacacagcacatttgaagacagcgaaagcaggagagactcactgtttgtgccgcacagacatggagagcgacgcaacagtaacggcaccaccactgaaacggaagtcagaaagagaaggttaagctcttaccagatttcaatggagatgctggaggattcctctggaaggcaaagagccgtgagcatagccagcattctgaccaacacaatggaagaacttgaagaatctagacagaaatgtccgccatgctggtatagatttgccaatgtgttcttgatctgggactgctgtgatgcatggttaaaagtaaaacatcttgtgaatttaattgttatggatccatttgttgatcttgccatcactatttgcattgtcttaaataccctctttatggccatggagcactaccccatgactgagcaattcagtagtgtgttgactgtaggaaacctggtctttactgggattttcacagcagaaatggttctcaagatcattgccatggatccttattactatttccaagaaggctggaatatctttgatggaattattgtcagcctcagtttaatggagcttggtctgtcaaatgtggagggattgtctgtactgcgatcattcagactgcttagagttttcaagttggcaaaatcctggcccacactaaatatgctaattaagatcattggcaattctgtgggggctctaggaaacctcaccttggtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggccaaaccatgtgccttattgttttcatgttggtcatggtcattggaaaccttgtggttctgaacctctttctggccttattattgagttcatttagctcagacaaccttgctgctactgatgatgacaatgaaatgaataatctgcagattgcagtaggaagaatgcaaaagggaattgattatgtgaaaaataagatgcgggagtgtttccaaaaagccttttttagaaagccaaaagttatagaaatccatgaaggcaataagatagacagctgcatgtccaataatactggaattgaaataagcaaagagcttaattatcttagagatgggaatggaaccaccagtggtgtaggtactggaagcagtgttgaaaaatacgtaatcgatgaaaatgattatatgtcattcataaacaaccccagcctcaccgtcacagtgccaattgctgttggagagtctgactttgaaaacttaaatactgaagagttcagcagtgagtcagaactagaagaaagcaaagagaaattaaatgcaaccagctcatctgaaggaagcacagttgatgttgttctaccccgagaaggtgaacaagctgaaactgaacccgaagaagactttaaaccggaagcttgttttactgaagggtgtattaaaaagtttccattctgtcaagtaagtacagaagaaggcaaagggaagatctggtggaatcttcgaaaaacctgctacagtattgttgagcacaactggtttgagactttcattgtgttcatgatccttctcagtagtggtgcattggcctttgaagatatatacattgaacagcgaaagactatcaaaaccatgctagaatatgctgacaaagtctttacctatatattcattctggaaatgcttctcaaatgggttgcttatggatttcaaacatatttcactaatgcctggtgctggctagatttcttgatcgttgatgtttctttggttagcctggtagccaatgctcttggctactcagaactcggtgccatcaaatcattacggacattaagagctttaagacctctaagagccttatcccggtttgaaggcatgagggtggttgtgaatgctcttgttggagcaattccctctatcatgaatgtgctgttggtctgtctcatcttctggttgatctttagcatcatgggtgtgaatttgtttgctggcaagttctaccactgtgttaacatgacaacgggtaacatgtttgacattagtgatgttaacaatttgagtgactgtcaggctcttggcaagcaagctcggtggaaaaacgtgaaagtaaactttgataatgttggcgctggctatcttgcactgcttcaagtggccacatttaaaggctggatggatattatgtatgcagctgttgattcacgagatgttaaacttcagcctgtatatgaagaaaatctgtacatgtatttatactttgtcatctttatcatctttgggtcattcttcactctgaatctattcattggtgtcatcatagataacttcaaccagcagaaaaagaagtttggaggtcaagacatctttatgacagaggaacagaaaaaatattacaatgcaatgaagaaacttggatccaagaaacctcagaaacccatacctcgcccagcaaacaaattccaaggaatggtctttgattttgtaaccagacaagtctttgatatcagcatcatgatcctcatctgcctcaacatggtcaccatgatggtggaaacggatgaccagggcaaatacatgaccctagttttgtcccggatcaacctagtgttcattgttctgttcactggagaatttgtgctgaagctcgtttccctcagacactactacttcactataggctggaacatctttgactttgtggtggtgattctctccattgtaggtatgtttctggctgagatgatagaaaagtattctgtgtcccctaccttgttccgagtgatccgtcttgccaggattggccgaatcctacgtctgatcaaaggagcaaaggggatccgcacgctgctctttgctttgatgatgtcccttcctgcgttgtttaacatcggcctcctgctcttcctggtcatgtttatctatgccatctttgggatgtccaactttgcctatgttaaaaaggaagctggaattgatgacatgttcaactttgagacctttggcaacagcatgatctgcttgttccaaattacaacctctgctggctgggatggattgctagcacctattcttaatagtgcaccacccgactgtgaccctgacacaattcaccctggcagctcagttaagggagaccgtggggacccatctgttgggattttcttttttgtcagttacatcatcatatccttcctggttgtggtgaacatgtacatcgcggtcatcctggagaacttcagtgttgctactgaagaaagtgcagagcccctgagtgaggatgactttgagatgttctatgaggtttgggaaaagtttgatcccgatgcgacccagtttatagagttctctaaactctctgattttgcagctgccctggatcctcctcttctcatagcaaaacccaacaaagtccagcttattgccatggatctgcccatggtcagtggtgaccggatccactgtcttgatattttatttgcctttacaaagcgtgttttgtgtgagagtggagagatggatgcccttcgaatacagatggaagacaggtttatggcatcaaacccctccaaagtctcttatgagcctattacaaccactttgaaacgtaaacaagaggaggtgtctgccgctatcattcagcgtaatttcagatgttatcttttaaagcaaaggttaaaaaatatatcaagtaactataacaaagaggcaattaaagggaggattgacttacctataaaacaagacatgattattgacaaactaaatgggaactccactccagaaaaaacagatgggagttcctctaccacctctcctccttcctatgatagtgtaacaaaaccagacaaggaaaagtttgagaaagacaaaccagaaaaagaaagcaaaggaaaagaggtcagagaaaatcaaaagtaaaaagaaacaaagaattatctttgtgatcaattgtttacagcctatgaaggtaaagtatatgtgtcaactggacttcaagaggaggtccatgccaaactgactgttttaacaaatactcatagtcagtgcctatacaagacagtgaagtgacctctctgtcactgcaactctgtgaagcagggtatcaacgttgacaagaggttgctgtttttattaccagctgacactgctgaggagaaacccaatggctacctagactatagggatagttgtgcaaagtgaacattgtaactacaccaaacacctttagtacagtccttgcatccattctatttttaacttccatatctgccatatttttacaaaatttgttctagtgcatttccatggtccccaattcatagtttattcataatgctatgtcactatttttgtaaatgaggtttacgttgaagaaacagtatacaagaaccctgtctctcaaatgatcagacaaaggtgttttgccagagagataaaatttttgctcaaaaccagaaaaagaattgtaatggctacagtttcagttacttccattttctagatggctttaattttgaaagtattttagtctgttatgtttgtttctatctgaacagttatgtgcctgtaaagtctcctctaatatttaaaggattatttttatgcaaagtattctgtttcagcaagtgcaaattttattctaagtttcagagctctatatttaatttaggtcaaatgctttccaaaaagtaatctaataaatccattctagaaaaatatatctaaagtattgctttagaatagttgttccactttctgctgcagtattgctttgccatcttctgctctcagcaaagctgatagtctatgtcaattaaataccctatgttatgtaaatagttattttatcctgtggtgcatgtttgggcaaatatatatatagcctgataaacaacttctattaaatcaaatatgtaccacagtgtatgtgtcttttgcaagcttccaacagggatgtatcctgtatcattcattaaacatagtttaaaggctatcactaatgcatgttaatattgcctatgctgctctattttactcaatccattcttcacaagtcttggttaaagaatgtcacatattggtgatagaatgaattcaacctgctctgtccattatgtcaagcagaataatttgaagctatttacaaacacctttacttttgcacttttaattcaacatgagtatcatatggtatctctctggatttcaaggaaacacactggatactgcctactgacaaaacctattcttcatattttgctaaaaatatgtctaaaacttgtttaaatataaataatgtaaaaatataatcaactttatttgtcagcattttgtacataagaaaattattttcaggttgatgacatcacaatttattttactttatgcttttgcttttgatttttaatcacaattccaaacttttgaatccataagatttttcaatggataatttcctaaaataaaagttagataatgggttttatggatttctttgttataatatattttctaccattccaataggagatacattggtcaaacactcaaacctagatcattttctaccaactatggttgcctcaatataaccttttattcatagatgtttttttttattcaacttttgtagtatttacgtatgcagactagtcttatttttttaattcctgctgcactaaagctattacaaatataacatggactttgttctttttagccatgaacaaagtggcaaagttgtgcaattacctaacatgatataaatttttgttttttgcacaaaccaaaagtttaatgttaattctttttacaaaactatttactgtagtgtattgaagaactgcatgcagggaattgctattgctaaaaagaatggtgagctacgtcattattgagccaaaagaataaatttcattttttattgcatttcacttattgggctctggggttttttgtttttgttttttgctgttggcagtttaaaatatatataattaataaaacctgtgcttgatctgacatttgtatacataaaagtttacatgaattttacaacaaactagtgcatgattcaccaagcagtactacagaacaaaggcaaattaaaagcagctttgtgaacttttatgtgtgcaaaggatcaagttcacatgttccaactttcaggtttgataataatagtagtaaccacctacaatagctttcaatttcaattaactcccttggctataagcatctaaactcatcttctttcaatataattgatgctatctcctaattacttggtggctaataaatgttacattctttgttacttaaatgcattatataaactcctatgtatacataaggtattaatgatatagttattgagaatttatattaacttttttttcaagaacccttggatttatgtgaggtcaaaaccaaactcttattctcagtggaaaactccagttgtaatgcatatttttaaagacaatttggatctaaatatgtatttcataattctcccataataaattatataaggtggaaaaaaaaaaaaaaaaaaaaaaaaaaaaa

SEQ ID NO: 7 - protein, 1951 aa, Homo sapiens [sequence omitted]

SEQ ID NO: 8 - DNA, 6822 bp, Rattus norvegicus
cagtgttttgtcgtttgcgcaatggcgtgtgtctgccagtagatggcagtgacacgttgagtgccgccaaccttttcttttttctttctttttttttttttttccccttccagggccgttttctgatatatgttgggtaccatagagtgaatctcagaacaggaagcggaggcataagcagagaggattccggaaaggtctctttgttttcatgtccacagagaaagcaagggggaaaaattgaatgtaatttgcaaatccctgtggcccaaatctgaagaactacagggggtggcaccgtccattctaaccatcttggatgctgtcctttgttgagctgtgattcctaaggctctccatcaggcaattcttatgcaagaagctaaacgtaattaaatgtgcaggatgaaaagatggcccaggcactgctggtacccccgggacctgagagcttccgccttttcactcgagaatctcttgctgctatcgaaaagcgtgctgcagaagagaaagccaagaaacccaagaaagagcaagacattgacgatgagaacaaaccaaagccaaacagcgacttggaagctgggaagaaccttccatttatctatggagacattcctccagagatggtgtcagagcccctggaggacctggacccctactatgtcagtaagaaaacttttgtagtgttgaataaagggaaggcgatttttcgattcagcgccacctccgccctgtatattttaactccgctaaaccctgttaggaaaattgccattaagattttggtacactctttgttcagcatgcttatcatgtgcactattttgaccaactgtgtatttatgacgttgagtaatcctcccgactggacaaagaatgtagagtatacgttcactgggatctatacctttgagtcacttataaagatcttggcaagagggttttgcttagaagatttcactttcctccgtgacccatggaactggctggatttcagtgtcatcgtgatggcatatgtgacagagtttgtggacctgggcaatgtctcagcgctgagaacgttcagagttctccgagcattgaaaacaatatcagtcattccaggtttaaagaccatcgtgggggccctgatccagtccgtgaagaagctgtccgacgtcatgatcctcaccgtgttctgtctcagtgtctttgctctaatcgggctgcagctcttcatgggcaacctgaggaataaatgctcgcagtggcccccgagcgattcggcttttgaaaccaacactacttcctacttcaatggcacaatggattcaaatgggacatttgttaatgtaacaatgagcactttcaactggaaggattatatcgcagatgacagtcacttttatgtcttggatggacaaaaagatcctttactctgtggaaatggctccgatgcaggacaatgtccagaagggtacatctgtgtgaaggctggacgaaaccccaactacggctacacaagctttgacaccttcagctgggccttcttgtccctgtttcgactcatgactcaggactactgggagaatctttaccagttgacattgcgtgcagctgggaaaacctacatgatatttttcgtcctggtaattttcttgggctcgttttatttggtgaacttgatcctggctgtggtggccatggcctatgaggagcagaaccaggccacactggaggaggctgaacagaaggaggcagagtttcagcagatgctggagcaactgaagaagcagcaggaggaggctcaggcagtggctgcagcctccgcggcatccagagacttcagtggaataggagggttaggagaacttctggagagttcttcagaagcttccaagttgagctccaagagtgctaaggagtggaggaaccggaggaagaagaggagacagagggaacacttggagggaaaccacagagccgatggagacaggtttcccaagtcggaatcggaagacagtgtcaaacgaagaagcttcctgctctccctggatggcaacccgctgactggtgacaagaagctgtgctctccccaccagtctctcttgagtatccgtggctccctgttttccccaagacgcaatagcaaaacgagcattttcagcttcagaggtcgggcgaaggacgtggggtctgagaatgactttgcagacgatgagcacagcaccttcgaggacagcgagagcaggagagactccctgtttgtgccgcacagacctggagagcgacgcaacagtaacggtaccaccactgaaacggaagtcaggaagagaaggctaagttcttaccagatttcaatggaaatgctggaggattcctctggaagacaaagatccatgagcatagccagtatcctgaccaacaccatggaggaacttgaagaatctagacagaagtgcccaccatgctggtatagattcgccaatgtgtttttgatctgggactgctgtgatgcatggttaaaagtgaagcatcttgtgaatttaattgtgatggatccatttgttgatcttgccataacaatttgcatcgtattaaatacactgttcatggccatggagcactatcccatgacccagcagttcagcagtgtgctgactgtgggaaacctggtcttcactgggatcttcacagccgaaatggtccttaaaatcattgccatggacccctattattatttccaagagggctggaatattttcgatggaattattgttagcctgagtttaatggagctaggcctggcaaatgtggaggggctgtctgtgcttcggtccttcagactgctccgagtcttcaagttggcaaagtcctggcccacactgaacatgctcattaagatcatcggcaactcggtgggcgcactgggcaacctgaccctggtgctggccatcatcgtcttcatttttgccgtggtcggcatgcagctgtttggaaagagctacaaggagtgtgtctgcaagatcaatgtggactgcaagctgccgcgctggcacatgaacgacttcttccactccttcctgatcgtgttccgagtgctgtgtggggagtggatagagaccatgtgggactgcatggaggtcgcgggccagaccatgtgccttattgtgttcatgttggtcatggtgattgggaaccttgtggttctgaacctctttctggccttattgttgagttcctttagttcagataaccttgctgctactgacgatgataacgaaatgaacaacctccagatcgcggtgggaaggatgcaaaagggaattgattttgtgaaaaataagatacgggagtgcttccgaaaagcgtttttcagaaagccgaaagtgatagaaatccaagaaggcaacaaaatagacagctgcatgtccaataacacgggcatcgaaataagcaaagagcttaactaccttaaagacggtaatggaaccaccagcggcgtgggaaccggaagcagtgtggaaaaatacgtaatcgatgaaaatgactacatgtcattcataaacaatcccagcctcaccgtgactgtgccaattgctgtgggagagtctgactttgaaaatttaaatacggaagagttcagcagtgagtcagaattggaagaaagtaaggagaaattaaatgcaaccagctcttctgaaggaagcacagttgatgttgctccaccccgagaaggtgaacaagcagaaattgaacctgaggaggaccttaagccagaagcttgttttactgaagggtgcattaaaaaattccccttctgtcaagtaagtacagaagaaggtaaaggaaaaatatggtggaatcttaggaagacatgctacagcattgtggagcacaactggtttgagacattcattgtgttcatgattctcctcagtagtggcgctttggcctttgaggatatatacattgagcaacgaaagacgatcaagaccatgctggagtatgcagacaaggtcttcacgtacatcttcatcctggagatgctcctcaaatgggtggcctatggatttcaaacctatttcaccaatgcctggtgctggttggacttcctgatcgttgatgtttctttggttagcctggtagccaatgctcttggttactcagaacttggtgccatcaaatccctacggacactgagagctctgaggccgctccgagccttatcccgctttgaaggcatgagggtggttgtaaatgctcttgttggtgcaattccctccatcatgaatgtgttattggtgtgtctcatcttctggctgatttttagcatcatgggtgtgaatctgtttgctggaaagttctatcactgtgttaacacgacaacaggcaacatgtttgaaataaaagaagtgaacaatttcagtgactgtcaggctcttggcaagcaagcccggtggaagaatgtgaaagtcaactttgacaacgttggggctggctacctggcattgctgcaagtggccacattcaaaggctggatggacatcatgtatgcagctgttgattcgcgggacgtcaaactgcagcccatatatgaagaaaacctgtacatgtacctgtactttgtcatcttcatcatcttcggctcgttcttcactctaaatctattcatcggtgtcatcatagacaacttcaaccagcagaagaagaagtttggaggtcaagacatctttatgacagaagaacagaagaaatactacaatgcaatgaagaagctcggctcaaagaaacctcagaagcccatccctcggcctgcaaacaaatttcaagggatggtctttgattttgtaaccagacaagtgtttgacatcagcatcatgatcctcatctgcctcaacatggtgaccatgatggtggaaacggatgaccagagcaaatacatgaccctggttttgtcccgaatcaacctagtgttcattgtcctcttcactggggagtttctgctgaagctcatctccctcagatactactacttcacgatagggtggaacatctttgactttgtggtggtgattctctcgattgtaggaatgtttctcgcagagctgatagagaagtatttcgtgtcccctaccctgttccgagtcatccgcctggccaggattggacgaatcctacgcctgatcaaaggcgccaaggggatccgcactctgctctttgctttgatgatgtcccttcctgcgctgttcaacatcggcctcctgcttttcctggtcatgttcatctacgccatctttgggatgtccaactttgcctatgttaaaaaagaggctggaattgatgacatgttcaactttgagacttttggcaacagcatgatctgcttgttccaaatcaccacctctgccggctgggacggactgctggcccccatcctcaacagcgcacctcccgactgtgaccccgatgcaattcaccctggaagctcggtgaagggggactgtgggaacccatccgtggggattttcttttttgtcagctacatcatcatatccttcctggtggtggtgaacatgtacatcgctgtcatcctggagaacttcagcgtcgccaccgaagaaagtgcagagcccctgagtgaggacgactttgagatgttctacgaggtctgggagaagttcgaccctgacgccactcagttcatagagttctgcaagctttctgactttgcagctgccctggatcctcccctcctcatcgcaaagccaaacaaagtccagctcattgccatggacctgcccatggtgagtggagaccgcatccactgcctggacatcttgtttgcttttacaaagcgggtcctgggcgagagtggagagatggacgctcttcgaatccagatggaagatcgcttcatggcttccaacccctccaaggtctcttatgagcccattaccaccaccctgaaacggaaacaagaggaggtgtctgctgctatcattcagcgtaattatagatgttatcttttaaagcaacggttaaaaaacatatcgagtaaatacgacaaagagacaatcaagggaaggattgacttgcctataaaaggagatatggttattgacaaattgaatgggaattccaccccagaaaagacggatgggagttcctccacaacctctcctccttcctatgacagtgtaacaaaaccagataaggaaaagtttgagaaagacaaaccagaaaaagaaatcaaagggaaagaggtcagagagaatcaaaagtaaaaagagacaaagaaatgtctttgtaatcaattgtttacagcctctgaaggtaaagtatccgtgtcaactggactctaaggagaggtccatgccaaactgactgtttcaacaaatactcaaggtcagtgcctataccagacagtgacctctgtcactgccactctgtgagacagggtatcaacattgacaagaggttgctgcttccattaccagctgacactgctgaggagaactccattgtgcaagtgacccgtcatcatgcccccaaactccattagtacaacgctcctgtcatctatttttaacattcacatttgccatatttttacaaaatctgtcccagtgtatcttcctggtccccacttcatagtctgttcataatactatgtcactatttttgtaaatgaagtttacgttaagggaaaatatatatataagaatcccatgttgctaagtccacaagtttctccagtaatcataaaaaaatattttgcctgagagatgaaattattgctcaaaacaaaaaaaaataaattctaatgttaacagtttc

SEQ ID NO: 9 - protein, 1951 aa, Rattus norvegicus
MAQALLVPPGPESFRLFTRESLAAIEKRAAEEKAKKPKKEQDIDDENKPKPNSDLEAGKNLPFIYGDIPPEMVSEPLEDLDPYYVSKKTFVVLNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVDLGNVSALRTFRVLRALKTISVIPGLKTIVGALIQSVKKLSDVMILTVFCLSVFALIGLQLFMGNLRNKCSQWPPSDSAFETNTTSYFNGTMDSNGTFVNVTMSTFNWKDYIADDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGIGGLGELLESSSEASKLSSKSAKEWRNRRKKRRQREHLEGNHRADGDRFPKSESEDSVKRRSFLLSLDGNPLTGDKKLCSPHQSLLSIRGSLFSPRRNSKTSIFSFRGRAKDVGSENDFADDEHSTFEDSESRRDSLFVPHRPGERRNSNGTTTETEVRKRRLSSYQISMEMLEDSSGRQRSMSIASILTNTMEELEESRQKCPPCWYRFANVFLIWDCCDAWLKVKHLVNLIVMDPFVDLAITICIVLNTLFMAMEHYPMTQQFSSVLTVGNLVFTGIFTAEMVLKIIAMDPYYYFQEGWNIFDGIIVSLSLMELGLANVEGLSVLRSFRLLRVFKLAKSWPTLNMLIKIIGNSVGALGNLTLVLAIIVFIFAVVGMQLFGKSYKECVCKINVDCKLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDFVKNKIRECFRKAFFRKPKVIEIQEGNKIDSCMSNNTGIEISKELNYLKDGNGTTSGVGTGSSVEKYVIDENDYMSFINNPSLTVTVPIAVGESDFENLNTEEFSSESELEESKEKLNATSSSEGSTVDVAPPREGEQAEIEPEEDLKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNTTTGNMFEIKEVNNFSDCQALGKQARWKNVKVNFDNVGAGYLALLQVATFKGWMDIMYAAVDSRDVKLQPIYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQSKYMTLVLSRINLVFIVLFTGEFLLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDAIHPGSSVKGDCGNPSVGIFFFVSYIIISFLVVVNMYIAVILENFSVATEESAEPLSEDDFEMFYEVWEKFDPDATQFIEFCKLSDFAAALDPPLLIAKPNKVQLIAMDLPMVSGDRIHCLDILFAFTKRVLGESGEMDALRIQMEDRFMASNPSKVSYEPITTTLKRKQEEVSAAIIQRNYRCYLLKQRLKNISSKYDKETIKGRIDLPIKGDMVIDKLNGNSTPEKTDGSSSTTSPPSYDSVTKPDKEKFEKDKPEKEIKGKEVRENQK

SEQ ID NO: 10 - DNA, 3494 bp, Mus musculus
tttttatatctgtgaggtgacgaaagaaaaagaaaaagaaaaagaagaaatgcaaataaaaaatgttgagccccagttaatgtcaggattttaatcaattcaattcttattggaggaactactttaaagcatgtagaagctgaggatagtggtcatatctctaacaccagtacttgggaggtgaagccagaagaactagcctagatttactctcagatatattgtaagtttgaggctagcctgggctacatgaaactgtatctcatccaacacataaaaaaagtttgatatatttggtatattaaacaatgatataacaaaatgtttagaattatagcaaaaataattggtaaaagtacctactatatatatatatatatatacatatatacatatatatgtatacatatatacatatatgtattttttaaatttaacataagcttttaaaaatcgaattatcttaccttttaaagagaaatgtaattcatatttcaacattcctttcagtttctagagtagaatctgaagtgacagttgatgcattagtagtgtctgttgtgtgcatctgtctgtcattccatccacgctttgcccacctgtaattagagaccctttcacacaggcaaggggcccagaggtctgcggcagtctgtaattaaagtattaatttttcttaggtcgatctaaaatgaaaataagtaaatctaaaattagatttataaagaaaaaaatgtgctgaaataacgacttgatttacaaccctaatattattatcacctttttgtttggaatctacattatgatcaactcataaaataatagtctagcttccaagtgtactacatttccttttatttcattacagctgcgagtcttcaagttggcaaaatcctggcccacactgaatatgctcattaagatcatcggcaactcggtgggcgcactgggcaacctgaccctggtgctggccatcatcgtcttcatttttgccgtggtcggcatgcagctgtttggaaagagctacaaggagtgtgtttgcaagatcaacgaggactgcaagctcccgcgctggcacatgaacgacttcttccactccttcctgatagtgttccgcgtgctgtgtggggagtggatagagaccatgtgggactgcatggaggtcgcgggccagaccatgtgccttattgtgtttatgttggtcatggtgattgggaaccttgtggttctgaaccttttcctggccttattgttgagttcctttagctcagacaaccttgctgctacggacgatgataacgaaatgaacaacctccagatcgcggtgggaaggatgcaaaaggggattgattatgtgaaaaataagatacgggagtgcttccgaaaagccttttttagaaagccgaaagtgatagaaatccacgaagggaacaaaatagacagctgcatgtccaataacacgggcgtagttgaaataagcaaagagcttaactaccttaaagacggtaacggaaccaccagtggcgtgggtactggaagcagtgtggagaaatacgtaattgatgaaaatgactacatgtcattcatcaacaaccccagcctcaccgtgacggtgccaattgccgtgggagagtctgactttgaaaatttaaacacggaagagtttagcagtgagtcagaactggaagaaagcaaggaggtaagcgcgcttttcagttcaggccaccacttttgcgcaatctacacagtatggctcctctgtattgttttttgcctgtgaaggtgcttaatcaacagaaagaaatgggttgggtgtgtagctcaagggataggatacttagcatgcatgacgtccttgtagtacccaacaccacacccaaaagcaacttaaacgtagataaagttggaacatcatgagaaatgggtaagttcgtttaaatataactttcccataggaaatcacaacttgagcgattaacatgttcattttcttgataactgaaagcctgatttaatatgcaatcctaaacaaaagagatactatttaacacatttttccctcgctagttatttgttttagaaaactcaatgaactatattttaatggtggtatgccatcactgtgaaaaatttacatagtctttattcacatttaagtgttttatttaccattttgtttactaggaagggtcaagccatactgatatttttcctattaaagatgagcaaacctatgacattttctacattaccttaaaaatactgaagattaacatttgatgattatagtggatctacagatgggaaaatttccgtcatatgagtagttgatcgaatgaaaagagaaaattttcatatgctatgatttgagctcatttgggtctaatgacaatagcttagtgacattaccttcaatattttattattatttatttatggcctttggatgtaacagctggaggttaatctagcacagatggtgttataaaggatctctagctggagggaatcctcaacaccctggaacagaccaatgatgtttgattctatggattctcttggagcttttgaaaaccagttacaccaagataatttgctctagaaatccttattagaagtttatggcatcacaactcacagtggtcccattcttttcatattttagaatatgttttaaaactaaaaaacatatgaacaaaatgaggtagcaatgtttattttcacagttgcacactaattgtgcatagcacatctaattcacgttgctcatcctagtagctaagactcaattctggctttattttctatcacctggcgtagaattgacttacaaaggaaccctagaggactagcaggcattcactgactattttgtccaattcggtcaaagacagtctttatcaagtggttcccaacaaactcataaagtcattaaaactctatttaattgatcttacaggccggtgttaaattcttaggcgtttaagaatgggaagggaggttaaatgacagaaaattgtaataggcctcatatttgggtcacatcgttaaaaagtggaaagatactgttctccaaaggagagccagggttgataatgagctcctgttactcaggcgagtggagagcaggagacagggcaaaggatgtgccggtgtgtctggaagctccagactgcagccttgcagagtaactcggagcttggaattgtctgcatggtcacgaaggagtgctctaaaacagaagcaattagaaaggggtatcctaggaacatctgttattaaagagtaaaggacgttagactgagagataatggagattaacttacacactgttatagcaatgagatggtgaaattctgggccaaaataatagagaaccaagcgtaattggcaatttaacagacctcaagaatctgaggaatcagaaagtggcaattccactgtctgataagggcttctgaggaaggtcaccaggtttgtatggaaaggaattc

SEQ ID NO: 11 - protein, 289 aa, Mus musculus
MLIKIIGNSVGALGNLTLVLAIIVFIFAVVGMQLFGKSYKECVCKINEDCKLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKIRECFRKAFFRKPKVIEIHEGNKIDSCMSNNTGVVEISKELNYLKDGNGTTSGVGTGSSVEKYVIDENDYMSFINNPSLTVTVPIAVGESDFENLNTEEFSSESELEESKEVSALFSSGHHFCAIYTVWLLCIVFCL

(19) United States
(12) Patent Application Publication
(10) Pub. No.: US 2008/0069773 A1
(43) Pub. Date: Mar. 20, 2008

(54) Novel Sodium Channel

(75) Inventors: Rodrigo Franco, Westford, MA (US); Angela Hornsten, Stow, MA (US)

Correspondence Address: KIRKPATRICK & LOCKHART PRESTON GATES ELLIS LLP;(FORMERLY KIRKPATRICK&LOCKHART NICHOLSON GRAHAM), STATE STREET FINANCIAL CENTER, ONE LINCOLN STREET, BOSTON, MA, 02111-2950, US

(73) Assignee: WYETH, Madison, NJ (US)

(21) Appl. No.: 10/582,531

(22) PCT Filed: Dec. 10, 2004
(86) PCT No.: PCT/US04/41668

§ 371 Date: Jun. 21, 2007

Related U.S. Application Data
(60) Provisional application No. 60/529,404, filed on Dec. 12, 2003.

(51) Int. Cl. C07K 14/435; A61K 45/00; A61K 49/00; C12N 1/00; C12N 15/63; G01N 33/68; C12N 5/00; C12N 15/12; A61P 25/00; C07H 21/04

(52) U.S. Cl. 424/9.2; 435/243; 435/320.1; 435/375; 436/86; 514/789; 530/350; 536/23.1; 536/23.5

(57) ABSTRACT

Novel sodium channel nucleic acids and polypeptides are disclosed herein. Methods of using the novel nucleic acids and polypeptides are also disclosed.

### CROSS-REFERENCE TO RELATED APPLICATIONS

This application claims priority to U.S. Provisional Application Ser. No. 60/529,404, filed Dec. 12, 2003, the contents of which are expressly incorporated herein by reference.

Each of the applications and patents cited in this text, as well as documents or references cited in each of the applications and patents (including during the prosecution of each issued patent; “application cited document”) and each of the PCT and foreign applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference. Documents incorporated by reference into this text or any teaching therein can be used in the practice of this invention.

### BACKGROUND

All cells rely on the regulated movement of inorganic ions across cell membranes to perform essential physiological functions. Electrical excitability, synaptic plasticity, and signal transduction are examples of processes in which changes in ion concentration play a important role. In general, the ion channels that permit these changes are proteinaceous pores consisting of one or multiple subunits, each containing two or more membrane-spanning domains. Most ion channels have selectivity for specific ions, primarily Na⁺, K⁺, Ca²⁺, or Cl⁻, by virtue of physical preferences for size and charge. Electrochemical forces, rather than active transport, drive ions across membranes, thus a single channel may allow the passage of millions of ions per second. Channel opening, or “gating” is tightly controlled by changes in voltage or by ligand binding, depending on the subclass of channel. Ion channels are attractive therapeutic targets due to their involvement in many physiological processes, yet the generation of drugs with specificity for particular channels in particular tissue types remains a major challenge.

Voltage-gated ion channels open in response to changes in membrane potential. For example, depolarization of excitable cells such as neurons result in a transient influx of Na⁺ ions, which propagates nerve impulses. This change in Na⁺ concentration is sensed by voltage-gated K⁺ channels which then allow an efflux of K⁺ ions. The efflux of K⁺ ions repolarizes the membrane. Other cell types rely on voltage-gated Ca²⁺ channels to generate action potentials. Voltage-gated ion channels also perform important functions in non-excitable cells, such as the regulation of secretory, homeostatic, and mitogenic processes. Ligand-gated ion channels can be opened by extracellular stimuli such as neurotransmitters (e.g., glutamate, serotonin, acetylcholine), or intracellular stimuli (e.g., cAMP, C²⁺, and phosphorylation).

Sodium (Na⁺) channels include voltage-gated and non-voltage-gated classes. Voltage-gated Na⁺ channels mediate a transient inward flow of Na⁺ ions required for regeneration of action potential in neurons. Voltage-gated Na⁺ channels can be further subdivided into subtypes; at least nine unique voltage-gated Na⁺ channel α subunits (Naᵥ1.1-1.9) have been cloned, and at least three associated β subunits have been cloned. The α subunits of voltage-gated Na⁺ channels are similar to the α subunits of voltage-gated Ca²⁺ channels, and contain four repeat regions, each containing six transmembrane domains. Voltage-gated Na⁺ channels are expressed in brain, muscle, heart, spinal cord, uterus, and sensory neurons. Alpha subunits associate with auxiliary subunits that regulate the function of the channels for example, by modifying the gating of the α subunit.

Genetic or pharmacological perturbations in ion channel function can have dramatic clinical consequences. Long QT syndrome, epilepsy, cystic fibrosis, and episodic ataxia are a few examples of heritable diseases resulting from mutations in ion channel subunits. Toxic side affects such as arrhythmia and seizure which are triggered by certain drugs are due to interference with ion channel function (Sirois and Atchison, Neurotoxicology, 17(1):63-84, 1996; Keating, M. T., Science 272:681-685, 1996). Drugs that modulate ion channel activity have applications in treatment of many pathological conditions, including pain, hypertension, angina pectoris, myocardial ischemia, asthma, bladder overactivity, alopecia, pain, heart failure, dysmenorrhea, type II diabetes, arrhythmia, graft rejection, seizure, convulsions, epilepsy, stroke, gastric hypermotility, psychoses, cancer, muscular dystrophy, and narcolepsy (Coghlan, M. J., et al., J. Med. Chem. 44:1627-1653, 2001; Ackerman. M. J., and Clapham, D. E., N. Eng. J. Med. 336:1575-1586, 1997). The growing number of identified ion channels and further understanding of their complexity will assist in future efforts at therapies that modify ion channel function.

### SUMMARY

Novel Na⁺ channel subunit polypeptides, nucleic acids, and fragments of the polypeptides and nucleic acids are provided herein. Also provided are methods of using the novel subunit polypeptides, nucleic acids, and fragments thereof.

In one aspect, the invention features an isolated sodium channel type III α subunit (mNaᵥ1.3 α subunit) polypeptide, wherein the polypeptide includes the amino acid sequence of SEQ ID NO:2. In one embodiment, the polypeptide essentially consists of the amino acid sequence of SEQ ID NO:2.

In another aspect, the invention features an isolated mNaᵥ1.3 α subunit polypeptide including at least 10 contiguous amino acids of SEQ ID NO:2, wherein the polypeptide includes one or more of the following amino acids: isoleucine 289, proline 518, serine 728, serine 1355, asparagine 1909, threonine 1910, and valine 1921.

In another aspect, the invention features an isolated mNaᵥ1.3 α subunit nucleic acid molecule that encodes a polypeptide described herein, e.g., a sodium channel type III α subunit (mNaᵥ1.3 α subunit) polypeptide including the amino acid sequence of SEQ ID NO:2. In one embodiment, the nucleic acid includes the nucleotide sequence of SEQ ID NO:1. In one embodiment, the nucleic acid molecule essentially consists of the nucleotide sequence of SEQ ID NO:1. In one embodiment, the nucleic acid is an allele of the nucleic acid sequence of SEQ ID NO:1.

In another aspect, the invention features a fragment of a mNaᵥ1.3 α subunit nucleic acid molecule that encodes a polypeptide described herein, e.g., a sodium channel type III α subunit (mNaᵥ1.3 α subunit) polypeptide including the amino acid sequence of SEQ ID NO:2. In one embodiment, the fragment encodes one or more of the following amino acids: isoleucine 289, proline 518, serine 728, serine 1355, asparagine 1909, threonine 1910, and valine 1921.

In another aspect, the invention features an expression vector including a nucleic acid encoding a mNaᵥ1.3 α subunit described herein, or a fragment thereof, operably linked to a promoter.

In another aspect, the invention features a host cell including a nucleic acid encoding a mNaᵥ1.3 α subunit described herein, or a fragment thereof.

In another aspect, the invention features an agent which preferentially binds to a mNaᵥ1.3 α subunit polypeptide, wherein the polypeptide includes the amino acid sequence of SEQ ID NO:2.

In another aspect, the invention features an agent which binds selectively to the mNaᵥ1.3 α subunit polypeptide, wherein the polypeptide includes the amino acid sequence of SEQ ID NO:2, and wherein the agent does not bind to a sodium channel type I or type II α subunit polypeptide. In one embodiment, the agent is a small molecule, a nucleic acid, or a protein. In one embodiment, the agent modulates a mNaᵥ1.3 α subunit polypeptide activity. In one embodiment, the agent is an antibody or antigen-binding fragment thereof. In one embodiment, the antibody is a polyclonal antibody or a monoclonal antibody.

In another aspect, the invention features a pharmaceutical composition including: an agent that binds selectively to a mNaᵥ1.3 α subunit polypeptide, wherein the polypeptide comprises the amino acid sequence of SEQ ID NO:2; and a pharmaceutically acceptable carrier.

In another aspect, the invention features a method for modulating a mNaᵥ1.3 α subunit polypeptide activity in a cell, the method including: providing a sodium channel including a mNaᵥ1.3 α subunit polypeptide, wherein the mNaᵥ1.3 α subunit polypeptide includes the amino acid sequence of SEQ ID NO:2; contacting the channel with an amount of a mNaᵥ1.3 α subunit polypeptide modulator effective to modulate an activity of the mNaᵥ1.3 α subunit polypeptide. In one embodiment, the modulator is a small molecule, a nucleic acid, or a protein.

In another aspect, the invention features a method for identifying an agent that modulates the activity of a mNaᵥ1.3 α subunit polypeptide, the method including: providing a first sodium channel comprising a mNaᵥ1.3 α subunit polypeptide, wherein the a mNaᵥ1.3 α subunit polypeptide includes the amino acid sequence of SEQ ID NO:2; contacting the channel with a test compound; and evaluating an activity of the sodium channel, wherein a change in activity relative to a reference value is an indication that the compound is an agent that modulates the channel.

In one embodiment, the test compound is a small molecule, a peptide, or a nucleic acid. In one embodiment, the sodium channel is contained within a biological sample. In one embodiment, the channel is contacted with multiple test compounds.

In one embodiment, the sample comprises a cell membrane. In one embodiment, the sample comprises a cell. In one embodiment, the cell is a eukaryotic cell. In one embodiment, the cell is Xenopus oocyte. In one embodiment, the cell is a mammalian cell.

In one embodiment, the activity comprises regulation of sodium concentration. In one embodiment, the evaluating comprises detecting sodium flux. In one embodiment, the contacting occurs under conditions that, in the absence of the test compound, cause a first amount of sodium flux. In one embodiment, the evaluating comprises using a Na⁺ flux assay. In one embodiment, the assay uses patch clamp electrophysiology. In one embodiment, the assay uses two electrode voltage clamp electrophysiology. In one embodiment, the assay comprises using a sodium-sensitive dye. In one embodiment, the assay is a high-throughput assay.

In one embodiment, the method further includes the steps of: providing a second sodium channel comprising a mNaᵥ1.3 α subunit polypeptide, wherein the mNaᵥ1.3 α subunit polypeptide is different that the first mNaᵥ1.3α subunit polypeptide (e.g., the second sodium channel is other than a mNaᵥ1.3 α subunit polypeptide comprising the amino acid sequence of SEQ ID NO:2); contacting the second sodium channel with the test compound; evaluating the activity of the second sodium channel.

The method can further include comparing the activity of the first sodium channel in the presence of the test compound to the activity of the second sodium channel in the presence of the test compound. In one embodiment, a plurality of sodium channels are provided.

In another aspect, the invention features a method for identifying an agent useful in the treatment of a disorder related to sodium current modulation, the method including: providing a sodium channel comprising a mNaᵥ1.3 α subunit polypeptide wherein the polypeptide includes the amino acid sequence of SEQ ID NO:2; contacting the channel with a test compound; and evaluating an activity of the channel, wherein a change in activity relative to a reference value is an indication that the test compound is an agent useful in a disorder related to sodium current. In one embodiment, the disorder is pain, paraesthesia, stroke, head trauma, a neurodegenerative disorder, or a disorder related to hyperexcitability of neurons.

The method can further include administering the compound in vivo (e.g., using an animal model). The method can further include modifying the compound for use in vivo. The method can further include evaluating modulation of a related human Naᵥ1.3 α subunit polypeptide by the compound.

In another aspect, the invention features a method for treating a subject having a disorder related to sodium channel current, the method comprising: identifying an agent that selectively binds a mNaᵥ1.3 α subunit polypeptide, and administering to a subject in need of such treatment a pharmacological agent which is selective for a sodium channel comprising a mNaᵥ1.3 α subunit polypeptide.

In one embodiment, the disorder is pain, paraesthesia, stroke, head trauma, a neurodegenerative disorder, or a disorder related to hyperexcitability of neurons.

### BRIEF DESCRIPTION OF THE DRAWINGS

FIG. 1 depicts an alignment of the amino acid sequences of a novel murine Naᵥ1.3 α subunit amino acid sequence with two human Naᵥ1.3 α subunit sequences and a rat Naᵥ1.3 α subunit sequence.

FIG. 2 depicts an alignment of the amino acid sequences of a novel murine Naᵥ1.3 α subunit amino acid sequence with an amino acid sequence encoded by a partial nucleotide sequence found under GenBank® Acc. No. NM_{—}018732. The predicted amino acid sequence from clone mNav1.3 wild-type is aligned with the published partial mouse Nav1.3 protein (accession number NM_{—}018732). Identical residues are highlighted and the stop codons are indicated in grey. The partial mouse mNav1.3 protein aligns with the clone mNav1.3 from amino acids 853 to 1115. The region from 264-289 may represent an alternative spliced region of mNav1.3.

FIG. 3 is a graph depicting sodium currents in Xenopus oocytes transfected with a novel murine Naᵥ1.3 α subunit, and depolarized at a range of voltages.

FIG. 4 is a graph depicting the average current/voltage relationship in Xenopus oocytes transfected with a novel murine Naᵥ1.3 α subunit, which were depolarized at a range of voltages.

FIG. 5 depicts SEQ ID NOs:1-11.

### DETAILED DESCRIPTION

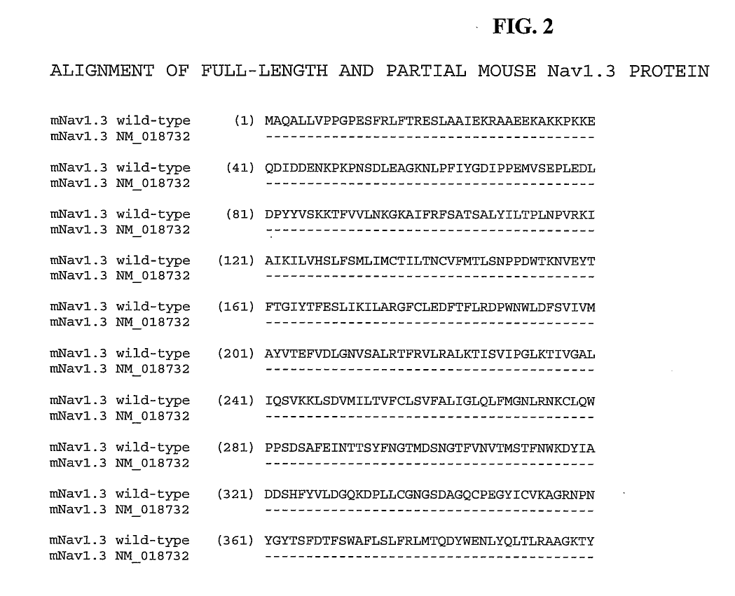

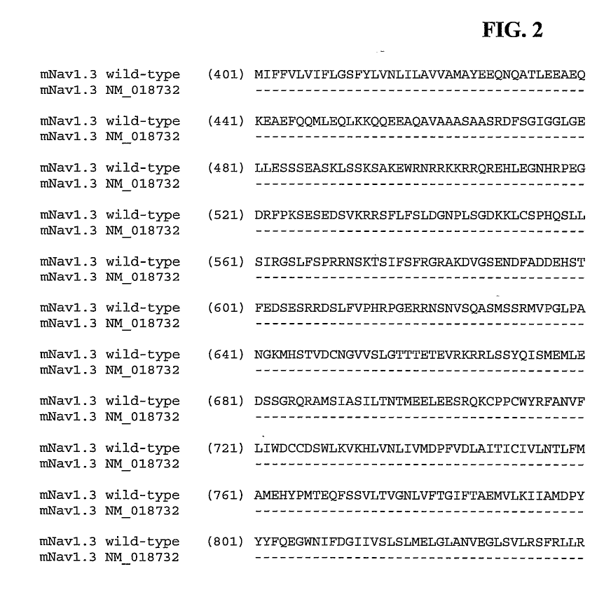

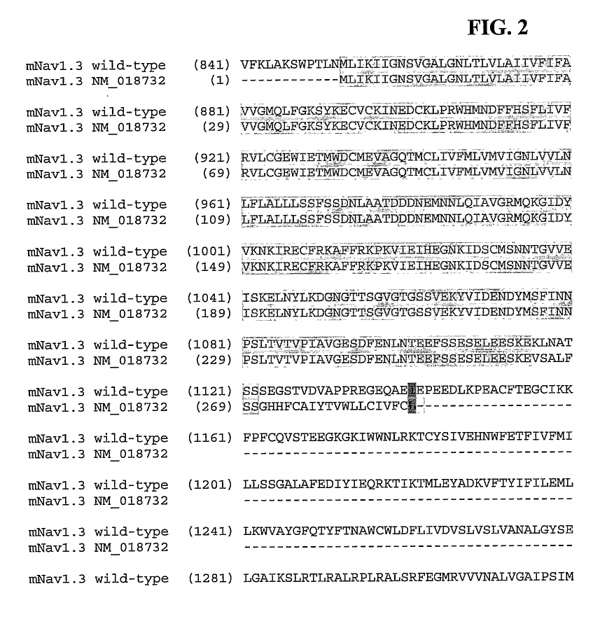

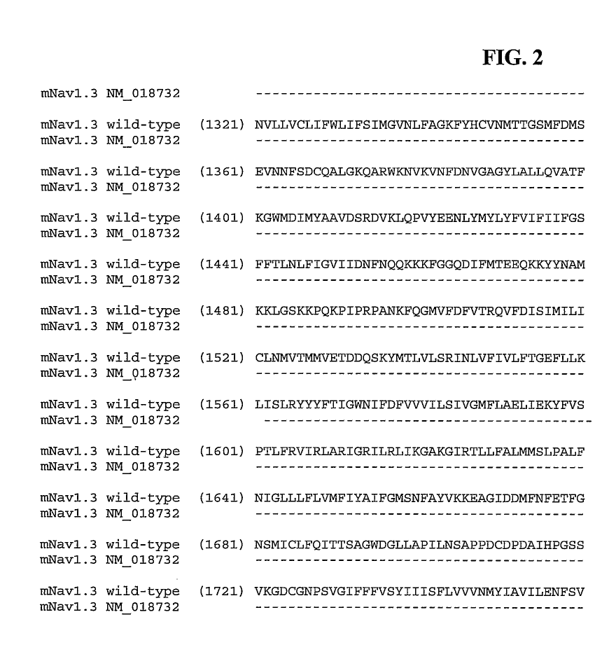

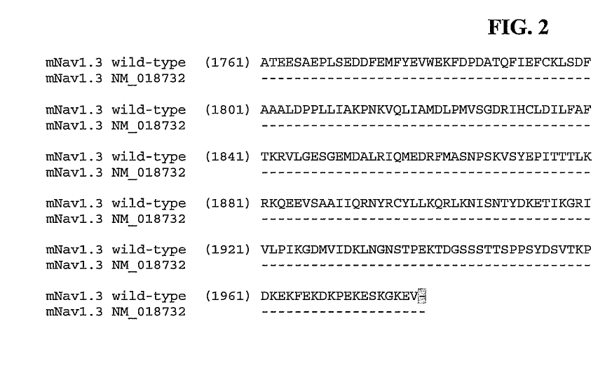

The invention is based, in part, on the identification of a novel Na⁺ channel type III α (Naᵥ1.3) subunit. The novel cDNA was isolated from murine brain tissue. The coding sequence of the cDNA (SEQ ID NO:1) corresponds to nucleotide 8 through nucleotide 5947 of the sequence shown in Table 1. The predicted amino acid sequence of the novel subunit is shown in SEQ ID NO:2. Related human and rat subunit nucleotide sequences are found in GenBank® under accession numbers NM_{—}006922, AF225986, and NM_{—}013119 (SEQ ID NO:4, SEQ ID NO:6, and SEQ ID NO:8). A fragment of a related murine Na⁺ channel cDNA has been cloned and is found under GenBank® accession no. NM_{—}018732 (SEQ ID NO:10). An alignment of SEQ ID NO:2 with this sequence is shown in FIG. 2.

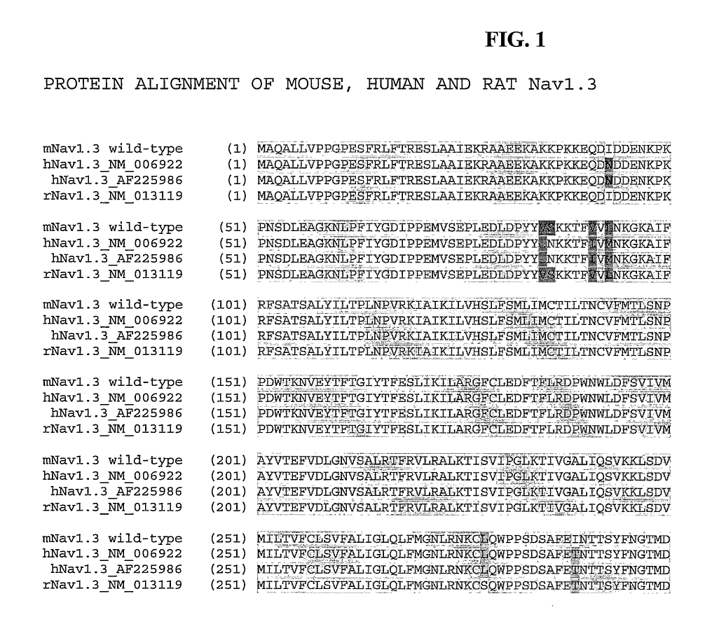

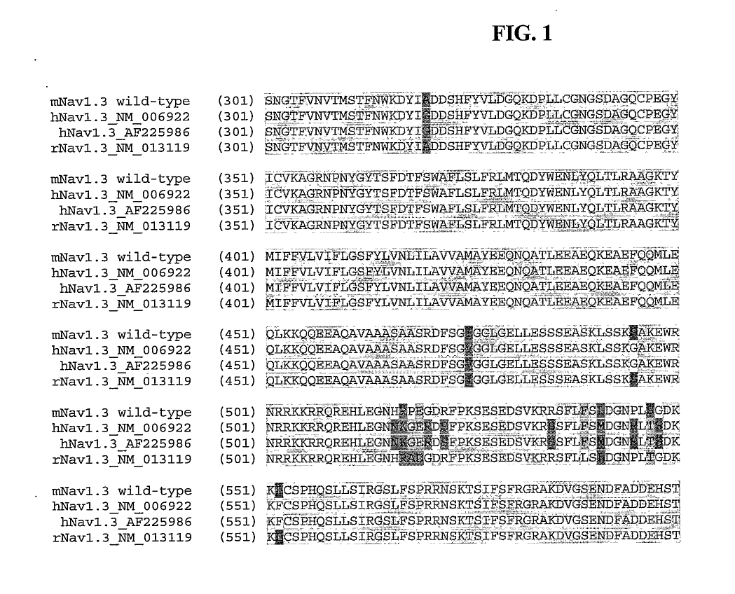

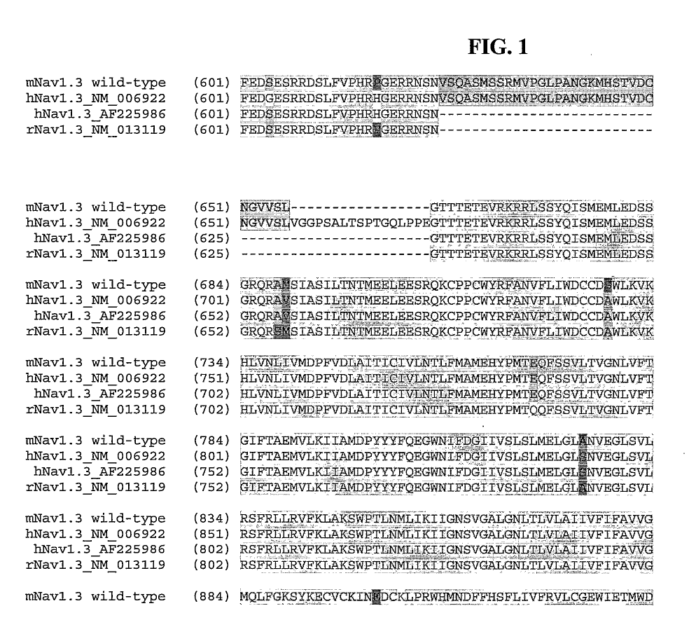

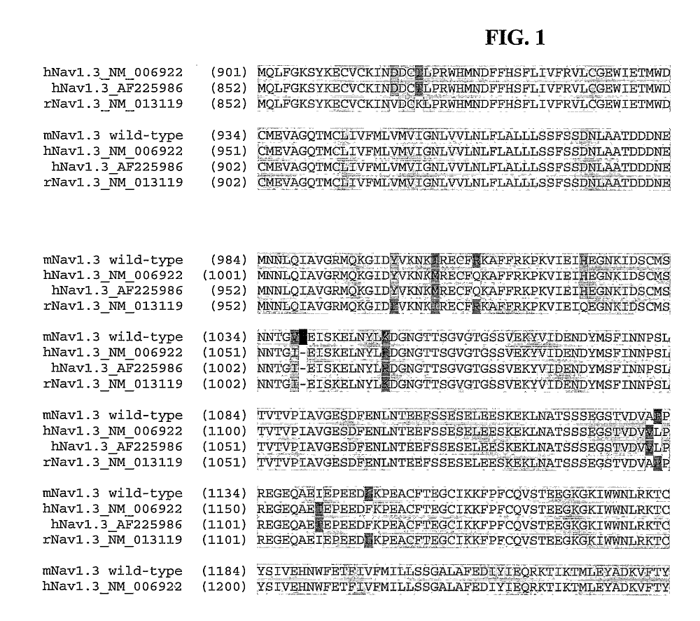

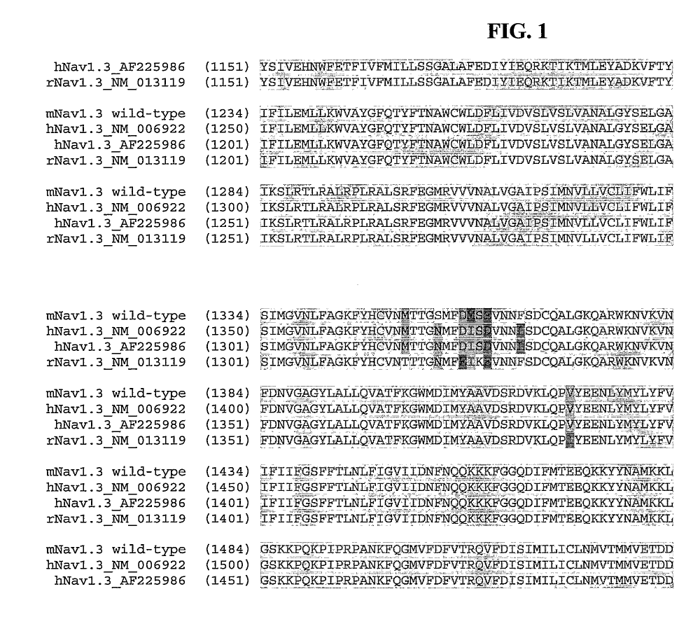

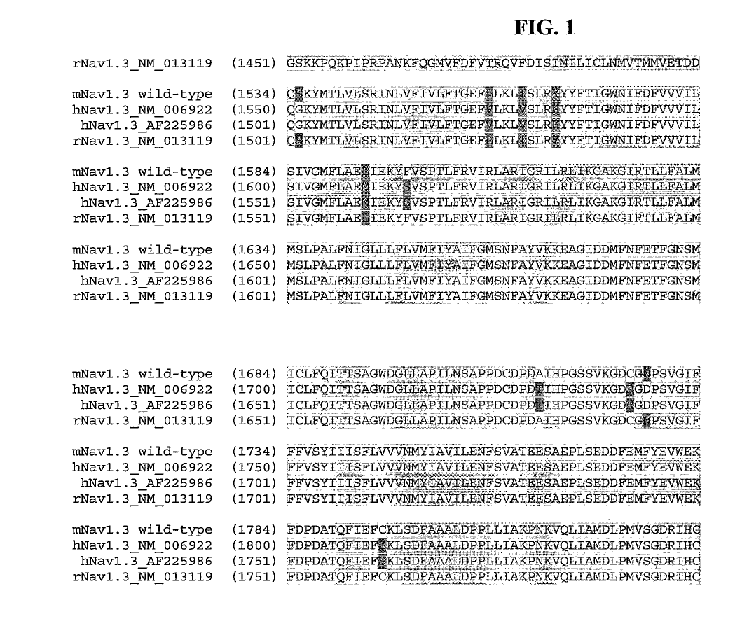

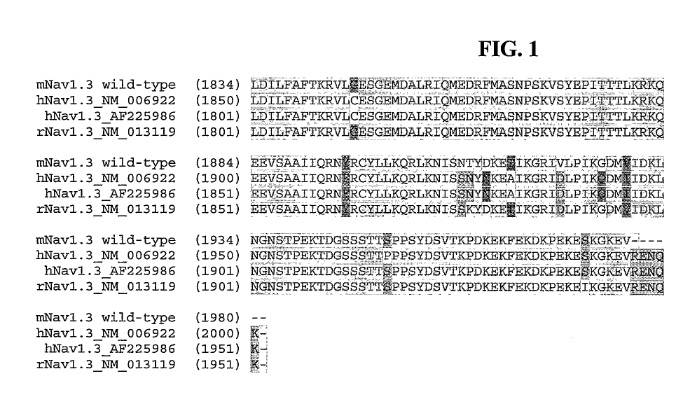

The novel murine nucleic acid sequence contains differences as compared to the related human and rat sequences. These differences are predicted to encode a unique polypeptide sequence. An alignment of the predicted amino acid sequence of the novel mouse Na⁺ channel α subunit and related rat and human subunits is depicted in FIG. 1.

### Voltage-Gated Na⁺ Channels

Voltage-gated Na⁺ channels are multisubunit transmembrane proteins having an α subunit of approximately 260 kilodaltons (kD), a β1 subunit of approximately 35 kD, and a β2 subunit of approximately 35 kD. These channels mediate the influx of sodium ions into cells. The α subunit forms the voltage-sensitive, pore-forming part of the channel, and the β subunits regulate gating of the α subunit and cell-cell interactions. The α subunit of Na⁺ channels contains four repeated domains (I-IV). Each of these repeated domains contains six transmembrane segments (S1-S6) (Reviewed in Baker, Md., and Wood, J N. Trends in Pharm Sci. 22(1):27-31, 2001).

Voltage-gated Na⁺ channels undergo cycles of resting (polarized; closed channel; activateable), open (depolarized; open channel; activated) and closed (depolarized, closed channel; inactivated) states in response to changes in membrane polarization. Most voltage-gated Na⁺ channels close rapidly (i.e., within milliseconds) of opening. Charged regions of the ion-conducting α subunit are sensitive to changes in membrane polarization.

Na⁺ channels enable production and propagation of electrical impulses in excitable cells. For example, Na⁺ channels expressed in primary sensory neurons (e.g., dorsal root ganglion (DRG) neurons, trigeminal neurons) produce a depolarizing upstroke in response to stimulation, which in turn is required for transmission of sensory information.

Subtypes of Na⁺ channels can be distinguished based on their sensitivity to inhibition by tetrodotoxin (TTX), a guanidinium toxin expressed by Puffer fish. TTX blocks the activity of Naᵥ1.1, Naᵥ1.2, and Naᵥ1.3 α subunit channels at concentrations in the nanomolar range. All three types of channels are expressed in brain. Disregulation of levels of Na⁺ channels in response to injury can lead to hyperexcitability of neuronal Na⁺ channels. This type of disregulation is thought to contribute to chronic pain. For example, axonal transection causes downregulation of certain Na⁺ channels and upregulation of Naᵥ1.3 α subunit channels (reviewed in Waxman, S. G., Nature Rev. 2:652-659, 2001), which leads to inappropriate repetitive firing of neurons.

As used herein, a “sodium channel α subunit” or “Na⁺ channel α subunit” refers to a protein which is involved in receiving, conducting, and transmitting signals, in a cell, such as a neuronal cell, e.g., a dorsal root ganglion. “Murine Naᵥ1.3 α subunit” or “mNaᵥ1.3 α subunit” refers to a murine type III α subunit described herein.

As used herein, a “Na⁺ channel mediated activity” refers to an activity, function, or response that involves a Na⁺ channel, e.g., a Na⁺ channel in a brain cell or a muscle cell. Na⁺ channel mediated activities are activities involved in receiving, conducting, and transmitting signals in, for example, the nervous system (e.g., the central nervous system and the peripheral nervous system), muscle tissue, cardiac tissue, and other cells and tissues. Na⁺ channel mediated activities include, for example, regulation of Na⁺ influx into cells and transmission of sensory stimuli. Also, as used interchangeably herein a “Na⁺ channel α subunit activity”, “mNaᵥ1.3 channel activity”, “biological activity of Na⁺ channel α subunit” or “functional activity of a Na⁺ channel α subunit”, refers to an activity, function, or response of a Na⁺ channel α subunit protein, polypeptide or nucleic acid molecule.

Isolated proteins of the present invention, e.g., mNaᵥ1.3 α subunit proteins described herein, have an amino acid sequence sufficiently homologous to the amino acid sequence of SEQ ID NO:2 or are encoded by a nucleotide sequence sufficiently homologous to SEQ ID NO:1. As used herein, the term “sufficiently homologous” refers to a first amino acid or nucleotide sequence which contains a sufficient or minimum number of identical or equivalent (e.g., an amino acid residue—which has a similar side chain) amino acid residues or nucleotides to a second amino acid or nucleotide sequence such that the first and second amino acid or nucleotide sequences share common structural domains or motifs or a common functional activity. All degenerate variants of the nucleotide sequence of SEQ ID NO:1 which encode SEQ ID NO:2 are considered to be “sufficiently homologous” to SEQ ID NO:1.

Accordingly, another embodiment of the invention features isolated mNaᵥ1.3 α subunit proteins and polypeptides, fragments, and variants thereof having mNaᵥ1.3 α subunit activity.

The novel mNaᵥ1.3 α subunit coding sequence (SEQ ID NO:1), which is approximately 5940 nucleotides in length, encodes a protein which is approximately 1980 amino acid residues in length. The gene encoding this novel subunit is expressed in the brain. Naᵥ1.3 channels are expressed, e.g., in brain, heart, and skeletal muscle. An Naᵥ1.3 channel can be expressed at low or high levels in a given tissue. Some Naᵥ1.3 channels expressed in neurons are upregulated following neuronal injury (Waxman, S G, et al. Brain Res. 8886:5-14, 2000; Kim, C H, et al. Mol. Brain. Res. 95:153-161, 2001).

Various aspects of the invention are described in further detail in the following subsections.

### Isolated Nucleic Acid Molecules

One aspect of the invention pertains to isolated nucleic acid molecules that encode mNaᵥ1.3 α subunit polypeptides or biologically active portions thereof, as well as nucleic acid fragments sufficient for use as hybridization probes to identify nucleic acid molecules that encode related isoforms of the mNaᵥ1.3 α subunit described herein and fragments for use as PCR primers for the amplification or mutation of mNaᵥ1.3 α subunit nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one that is separated from other nucleic acid molecules which are present in the natural source of the nucleic acid. An “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated mNaᵥ1.3 α subunit nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of SEQ ID NO:1, or a portion thereof, can be isolated using standard molecular biology techniques and the sequence information provided herein. Using all or a portion of the nucleic acid sequence of SEQ ID NO:1 as a hybridization probe, mNaᵥ1.3 α subunit nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to Na⁺ channel α subunit nucleotide sequences can be prepared by standard synthetic techniques, e.g., using an automated DNA synthesizer.

In another embodiment, an isolated nucleic acid molecule of the invention comprises a mNaᵥ1.3 α subunit nucleic acid molecule which is a complement of the nucleotide sequence shown in SEQ ID NO:1, or a portion of these nucleotide sequences. A nucleic acid molecule which is complementary to the nucleotide sequence shown in SEQ ID NO:1 is one which is sufficiently complementary to the nucleotide sequence shown in SEQ ID NO:1 such that it can hybridize to the nucleotide sequence shown in SEQ ID NO:1.

In still another embodiment, an isolated nucleic acid molecule of the present invention comprises a nucleotide sequence which is at least about 75%, 85%, 95% or more homologous to the entire length of the nucleotide sequence shown in SEQ ID NO:1, or a portion of this nucleotide sequence.

Moreover, the nucleic acid molecule of the invention can comprise only a portion of the nucleic acid sequence of SEQ ID NO:1, for example a fragment which can be used as a probe or primer or a fragment encoding a biologically active portion of a mNaᵥ1.3 α subunit protein. The nucleotide sequence determined from the cloning of the mNaᵥ1.3 α subunit cDNA allows for the generation of probes and primers designed for use in identifying and/or cloning related isoforms, as well as homologues from other species. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, of an anti-sense sequence of SEQ ID NO:1, or of a naturally occurring allelic variant or mutant of SEQ ID NO:1.

Probes based on the mNaᵥ1.3 α subunit nucleotide sequences can be used to detect transcripts encoding related isoforms. The probe can further include a label group attached thereto, e.g., the label group can be a radioisotope, a fluorescent compound, an enzyme, or an enzyme co-factor. Such probes can be used as a part of a diagnostic test kit for identifying cells or tissue which express or misexpress a mNaᵥ1.3 α subunit protein, such as by measuring a level of a mNaᵥ1.3 α subunit-encoding nucleic acid in a sample of cells from a subject e.g., detecting mNaᵥ1.3 α subunit mRNA levels or determining whether a genomic mNaᵥ1.3 α subunit gene has been mutated or deleted in a region that would affect expression of the mNaᵥ1.3 α subunit isoform.

A nucleic acid fragment encoding a “biologically active portion of a mNaᵥ1.3 α subunit protein” can be prepared by isolating a portion of the nucleotide sequence of SEQ ID NO:1, which encodes a polypeptide having a mNaᵥ1.3 α subunit biological activity (the biological activities of the mNaᵥ1.3 α subunit proteins are described herein), expressing the encoded portion of the mNaᵥ1.3 α subunit protein (e.g., by recombinant expression in vitro) and assessing the activity of the encoded portion of the mNaᵥ1.3 α subunit protein.

The invention further encompasses nucleic acid molecules that differ from the nucleotide sequence shown in SEQ ID NO:1, due to degeneracy of the genetic code and thus encode the same mNaᵥ1.3 α subunit proteins as those encoded by the nucleotide sequence shown in SEQ ID NO:1. In another embodiment, an isolated nucleic acid molecule of the invention has a nucleotide sequence encoding a protein having an amino acid sequence shown in SEQ ID NO:2.

In addition to the mNaᵥ1.3 α subunit nucleotide sequences shown in SEQ ID NO:1, it will be appreciated by those skilled in the art that DNA sequence polymorphisms that lead to changes in the amino acid sequences of the mNaᵥ1.3 α subunit proteins may exist within a population. Such genetic polymorphism in the mNaᵥ1.3 α subunit genes may exist among individuals within a population due to natural allelic variation. As used herein, the terms “gene” and “recombinant gene” refer to nucleic acid molecules comprising an open reading frame encoding a mNaᵥ1.3 α subunit protein. Such natural allelic variations can typically result in 1-5% variance in the nucleotide sequence of a mNaᵥ1.3 α subunit gene. Any and all such nucleotide variations and resulting amino acid polymorphisms in mNaᵥ1.3 α subunit genes that are the result of natural allelic variation and that do not alter the functional activity of a mNaᵥ1.3 α subunit protein are intended to be within the scope of the invention.

Moreover, nucleic acid molecules encoding other mNaᵥ1.3 α subunit channel family members and thus which have a nucleotide sequence which differs from the mNaᵥ1.3 α subunit sequences of SEQ ID NO:1 are intended to be within the scope of the invention. For example, another mNaᵥ1.3 α subunit cDNA can be identified based on the nucleotide sequence of a mNaᵥ1.3 α subunit (e.g., SEQ ID NO:1). Moreover, nucleic acid molecules encoding mNaᵥ1.3 α subunit proteins from different species, and thus which have a nucleotide sequence which differs from the mNaᵥ1.3 α subunit sequences of SEQ ID NO:1 are intended to be within the scope of the invention.

Nucleic acid molecules corresponding to natural allelic variants and homologues of the mNaᵥ1.3 α subunit cDNAs of the invention can be isolated based on their homology to the mNaᵥ1.3 α subunit nucleic acids disclosed herein using the cDNAs disclosed herein, or a portion thereof, as a hybridization probe according to standard hybridization techniques under stringent hybridization conditions.

Accordingly, in another embodiment, an isolated nucleic acid molecule of the invention is at least 15, 20, 25, 30 or more nucleotides in length and hybridizes under stringent conditions to the nucleic acid molecule comprising the nucleotide sequence of SEQ ID NO:1. Preferably, the molecule hybridizes under highly stringent conditions. In other embodiments, the nucleic acid is at least 30, 300, 500, 700, 850, 950, or 2000 nucleotides in length. As used herein, the term “hybridizes under stringent conditions” is intended to describe conditions for hybridization and washing under which nucleotide sequences at least 60%, 85%, or 95% homologous to each other typically remain hybridized to each other. Hybridization conditions are known to those skilled in the art and can be found in Current Protocols in Molecular Biology, John Wiley & Sons, N.Y., 6.3 α subunit.1-6.3 α subunit.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by a wash in 0.2×SSC, 0.1% SDS at 65° C.

An isolated nucleic acid molecule of the invention that hybridizes under moderate or highly stringent conditions to the sequence of SEQ ID NO:1 can correspond to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of the mNaᵥ1.3 α subunit sequences that may exist in the population, the skilled artisan will further appreciate that changes can be introduced by mutation into the nucleotide sequences of SEQ ID NO:1, thereby leading to changes in the amino acid sequence of the encoded mNaᵥ1.3 α subunit proteins, without altering the functional ability of the mNaᵥ1.3 α subunit proteins. For example, nucleotide substitutions leading to amino acid substitutions at “non-essential” amino acid residues can be made in the sequence of SEQ ID NO:1. A “non-essential” amino acid residue is a residue that can be altered from the wild-type sequence of mNaᵥ1.3 α subunit without altering the biological activity, whereas an “essential” amino acid residue is required for biological activity. For example, amino acid residues that are conserved among the mNaᵥ1.3 α subunit proteins of the present invention, are predicted to be particularly unamenable to alteration. Furthermore, additional amino acid residues that are conserved between the mNaᵥ1.3 α subunit proteins of the present invention and other mNaᵥ1.3 α subunit channel subunits are not likely to be amenable to alteration.

Accordingly, another aspect of the invention pertains to nucleic acid molecules encoding mNaᵥ1.3 α subunit proteins that contain changes in amino acid residues that are not essential for activity. Such mNaᵥ1.3 α subunit proteins differ in amino acid sequence from SEQ ID NO:2, yet retain biological activity. Biological activity can be measured by an assay described herein, e.g., a Na⁺ channel activity assay, e.g., a Na⁺ influx assay. In one embodiment, the isolated nucleic acid molecule comprises a nucleotide sequence encoding a protein, wherein the protein comprises an amino acid sequence at least about 65%, 75%, 85%, 95% or more homologous to SEQ ID NO:2.

An isolated nucleic acid molecule encoding a mNaᵥ1.3 α subunit protein homologous to the protein of SEQ ID NO:2 can be created by introducing one or more nucleotide substitutions, additions or deletions into the nucleotide sequence of SEQ ID NO:1 such that one or more amino acid substitutions, additions or deletions are introduced into the encoded protein. Mutations can be introduced into SEQ ID NO:1 by standard techniques, such as site-directed mutagenesis and PCR-mediated mutagenesis. Conservative amino acid substitutions can be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a mNaᵥ1.3 α subunit protein is preferably replaced with another amino acid residue from the same side chain family. Following mutagenesis of SEQ ID NO:1, the encoded protein can be expressed and the activity of the protein can be determined.

A mutant mNaᵥ1.3 α subunit protein can be assayed for the ability to (1) interact with a non mNaᵥ1.3 α subunit protein molecule, e.g., Na⁺ channel β1 or β2 subunits, or TTX; or (2) modulate membrane excitability.

In addition to the nucleic acid molecules encoding mNaᵥ1.3 α subunit proteins described above, another aspect of the invention pertains to isolated nucleic acid molecules that are antisense thereto. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire mNaᵥ1.3 α subunit coding strand, or to only a portion thereof. In one embodiment, an antisense nucleic acid molecule is antisense to a “coding region” of the coding strand of a nucleotide sequence encoding mNaᵥ1.3 α subunit. The term “coding region” refers to the region of the nucleotide sequence comprising codons which are translated into amino acid residues (e.g., the coding region of a mnaᵥ1.3 α subunit corresponds to SEQ ID NO:1). In another embodiment, the antisense nucleic acid molecule is antisense to a “noncoding region” of the coding strand of a nucleotide sequence encoding a mNaᵥ1.3 α subunit. The term “noncoding region” refers to 5′ and 3′ sequences that flank the coding region that are not translated into amino acids (i.e., also referred to as 5′ and 3′ untranslated regions).

Given the coding strand sequences encoding a mNaᵥ1.3 α subunit disclosed herein (e.g., SEQ ID NO:1), antisense nucleic acids of the invention can be designed according to the rules of Watson and Crick base pairing. The antisense nucleic acid molecule can be complementary to the entire coding region of mNaᵥ1.3 α subunit mRNA, but more preferably is an oligonucleotide which is antisense to only a portion of the coding or noncoding region of mNaᵥ1.3 α subunit mRNA. For example, the antisense oligonucleotide can be complementary to the region surrounding the translation start site of a mNaᵥ1.3 α subunit mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uraci 1,5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, and 2,6-diaminopurine. Alternatively, the antisense nucleic acid can be produced biologically using an expression vector into which a nucleic acid has been subdloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a mNaᵥ1.3 α subunit protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are envisioned.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. Nucleic Acids. Res. 15:6625-6641, 1987). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. FEBS Lett. 215:327-330, 1987).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach Nature 334:585-591, 1988)) can be used to catalytically cleave mNaᵥ1.3 α subunit mRNA transcripts to thereby inhibit translation of mNaᵥ1.3 α subunit mRNA. A ribozyme having specificity for a mNaᵥ1.3 α subunit-encoding nucleic acid can be designed based upon the nucleotide sequence of a mNaᵥ1.3 α subunit cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a mNaᵥ1.3 α subunit-encoding mRNA. See, e.g., Cech et al. U.S. Pat. No. 4,987,071; and Cech et al. U.S. Pat. No. 5,116,742. Alternatively, mNaᵥ1.3 α subunit mRNA can be used to select a catalytic RNA having a specific ribonuclease activity from a pool of RNA molecules. See, e.g., Bartel, D. and Szostak, J. W. Science 261:1411-1418, 1993.

In another embodiment of the present invention, RNA interference (RNAi) can be used to inhibit the expression of a mNaᵥ1.3 α subunit protein. Various inhibitory RNAi molecules can be identified and those that inhibit expression of a mNaᵥ1.3 α subunit can be formulated as pharmaceutical compositions to be administered in the methods of treatment described herein.

RNAi is a term used to refer to the mechanism by which a particular mRNA is degraded in host cells. To inhibit an mRNA, double-stranded RNA (dsRNA) corresponding to a portion of the gene to be silenced (e.g., a gene encoding a mNaᵥ1.3 α subunit polypeptide) is introduced into a cell. The dsRNA is digested into 21-25 nucleotide-long duplexes called short interfering RNAs (or siRNAs), which bind to a nuclease complex to form what is known as the RNA-induced silencing complex (or RISC). The RISC targets the homologous transcript by base pairing interactions between one of the siRNA strands and the endogenous mRNA. It then cleaves the mRNA about 12 nucleotides from the 3′ terminus of the siRNA (see Sharp et al., Genes Dev. 15:485-490, 2001, and Hammond et al., Nature Rev. Gen. 2:110-119, 2001).

RNAi has proven successful in human cells, including human embryonic kidney and HeLa cells (see, e.g., Elbashir et al., Nature 411:494-498, 2001). Gene silencing can be induced in mammalian cells by enforcing endogenous expression of RNA hairpins (see Paddison et al., Proc. Natl. Acad. Sci. USA 99:1443-1448, 2002) or by transfection of small (21-23 nt) dsRNA (reviewed in Caplen, Trends in Biotech. 20:49-51, 2002).

RNAi technology utilizes standard molecular biology methods. The dsRNA (which, here, for example, would correspond to the sequence encoding a mNaᵥ1.3 α subunit polypeptide) can be produced by standard methods (e.g., by simultaneously transcribing both strands of a template DNA corresponding to a mNaᵥ1.3 α subunit sequence with T7 RNA polymerase; the RNA can also be chemically synthesized or recombinantly produced). Kits for producing dsRNA are available commercially (from, e.g., New England Biolabs, Inc). The RNA used to mediate RNAi can include synthetic or modified nucleotides, such as phosphorothioate nucleotides. Methods of transfecting cells with dsRNA or with plasmids engineered to make dsRNA are also routine in the art.

Gene silencing effects similar to those observed with RNAi have been reported in mammalian cells transfected with an mRNA-cDNA hybrid construct (Lin et al., Biochem. Biophys. Res. Comm. 281:639-644, 2001). Accordingly, mRNA-cDNA hybrids containing mNaᵥ1.3 α subunit sequence, as well as duplexes that contain mNaᵥ1.3 α subunit sequence (e.g., duplexes containing 21-23 bp monomers), are within the scope of the present invention. The hybrids and duplexes can be tested for activity according to the assays described herein (i.e., they can serve as the test agents), and those that exhibit inhibitory activity can be used to treat patients who have, or who may develop, a disease or condition associated with a mNaᵥ1.3 α subunit activity, e.g., neuropathic pain.

The dsRNA molecules of the invention (double-stranded RNA molecules corresponding to portions of a mnaᵥ1.3 α subunit gene) can vary in a number of ways. For example, they can include a 3′ hydroxyl group and, as noted above, can contain strands of 21, 22, or 23 consecutive nucleotides. Moreover, they can be blunt ended or include an overhanging end at either the 3′ end, the 5′ end, or both ends. For example, at least one strand of the RNA molecule can have a 3′ overhang from about 1 to about 6 nucleotides (e.g., 1-5, 1-3, 2-4 or 3-5 nucleotides (whether pyrimidine or purine nucleotides) in length. Where both strands include an overhang, the length of the overhangs may be the same or different for each strand. To further enhance the stability of the RNA duplexes, the 3′ overhangs can be stabilized against degradation (by, e.g., including purine nucleotides, such as adenosine or guanosine nucleotides or replacing pyrimidine nucleotides by modified analogues (e.g., substitution of uridine 2 nucleotide 3′ overhangs by 2′-deoxythymidine is tolerated and does not affect the efficiency of RNAi). The single stranded mNaᵥ1.3 α subunit RNA molecules that make up the duplex or hybrid inhibitor, or that act simply as antisense RNA oligonucleotides, are also within the scope of the invention. Any dsRNA can be used in the methods of the present invention, provided it has sufficient homology to a target gene of interest, e.g., a mNaᵥ1.3 α subunit gene, to mediate RNAi. While duplexes having 21-23 nucleotides are described above, the invention is not so limited; there is no upper limit on the length of the dsRNA that can be used (e.g., the dsRNA can range from about 21 base pairs of the gene to the full length of the gene or more (e.g., 50-100, 100-250, 250-500, 500-1000, or over 1000 base pairs).

When these nucleic acids are administered to a human, they can reduce mNaᵥ1.3 α subunit mRNA levels, thereby inhibiting expression of a mNaᵥ1.3 α subunit. The cell or organism is maintained under conditions in which mNaᵥ1.3 α subunit mRNA is degraded, thereby mediating RNAi in the cell or organism. Alternatively, cells can be obtained from the individual, treated ex vivo, and re-introduced into the individual.

In yet another embodiment, the mNaᵥ1.3 α subunit nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. Bioorganic & Medicinal Chemistry 4 (1): 5-23, 1996). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. supra; Perry-O'Keefe et al. Proc. Natl. Acad. Sci. 93: 14670-675, 1996.

PNAs of mNaᵥ1.3 α subunit nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of mNaᵥ1.3 α subunit nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B., supra)); or as probes or primers for DNA sequencing or hybridization (Hyrup B. et al., supra; Perry-O'Keefe, supra).

In another embodiment, PNAs of mNaᵥ1.3 α subunit can be modified, (e.g., to enhance their stability or cellular uptake), by attaching lipophilic or other helper groups techniques of drug delivery known in the art.

In other embodiments, the oligonucleotide may include other appended groups such as peptides (e.g., for targeting host cell receptors in vivo), or agents facilitating transport across the cell membrane (see, e.g., Letsinger et al. Proc. Natl. Acad. Sci. US. 86:6553-6556, 1989; Lemaitre et al. Proc. Natl. Acad. Sci. USA 84:648-652, 1987; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. Bio-Techniques 6:958-976, 1988) or intercalating agents (See, e.g., Zon Pharm. Res. 5:539-549, 1988). To this end, the oligonucleotide may be conjugated to another molecule, (e.g., a peptide, hybridization triggered cross-linking agent, transport agent, or hybridization-triggered cleavage agent).

### Isolated mNaᵥ1.3 Proteins and Anti-mNaᵥ1.3 Antibodies

One aspect of the invention pertains to isolated mNaᵥ1.3 α subunit proteins, and biologically active portions thereof, as well as polypeptide fragments suitable for use as immunogens to raise anti-mNaᵥ1.3 α subunit antibodies. In one embodiment, native mNaᵥ1.3 α subunit proteins can be isolated from cells or tissue sources by an appropriate purification scheme using standard protein purification techniques. In another embodiment, mNaᵥ1.3 α subunit proteins are produced by recombinant DNA techniques. Alternative to recombinant expression, a mNaᵥ1.3 α subunit protein or polypeptide can be synthesized chemically using standard peptide synthesis techniques.

An “isolated” or “purified” protein or biologically active portion thereof is substantially free of cellular material or other contaminating proteins from the cell or tissue source from which the mNaᵥ1.3 α subunit protein is derived, or substantially free from chemical precursors or other chemicals when chemically synthesized. The language “substantially free of cellular material” includes preparations of mNaᵥ1.3 α subunit protein in which the protein is separated from cellular components of the cells from which it is isolated or recombinantly produced. In one embodiment, the language “substantially free of cellular material” includes preparations of mNaᵥ1.3 α subunit protein having less than about 30% (by dry weight) of non-mNaᵥ1.3 α subunit protein (also referred to herein as a “contaminating protein”), more preferably less than about 20%, 10%, or 5% of non-mNaᵥ1.3 α subunit protein. When the mNaᵥ1.3 α subunit protein or biologically active portion thereof is recombinantly produced, it is also preferably substantially free of culture medium, i.e., culture medium represents less than about 20%, 10%, or 5% of the volume of the protein preparation.

The language “substantially free of chemical precursors or other chemicals” includes preparations of mNaᵥ1.3 α subunit protein in which the protein is separated from chemical precursors or other chemicals that are involved in the synthesis of the protein. In one embodiment, the language “substantially free of chemical precursors or other chemicals” includes preparations of mNaᵥ1.3 α subunit protein having less than about 30%, 20%, 10%, or 5% (by dry weight) of chemical precursors or non-mNaᵥ1.3 α subunit chemicals.

As used herein, a “biologically active portion” of a mNaᵥ1.3 α subunit protein includes a fragment of a mNaᵥ1.3 α subunit protein which participates in an interaction between a mNaᵥ1.3 α subunit molecule and a non mNaᵥ1.3 α subunit molecule. Biologically active portions of a mNaᵥ1.3 α subunit proteins include peptides comprising amino acid sequences sufficiently homologous to or derived from the amino acid sequence of the mNaᵥ1.3 α subunit protein, e.g., the amino acid sequence shown in SEQ ID NO:2, which include less amino acids than the full length mNaᵥ1.3 α subunit proteins, and exhibit at least one activity of a mNaᵥ1.3 α subunit protein. Typically, biologically active portions comprise a domain or motif with at least one activity of the mNaᵥ1.3 α subunit protein, e.g., binding of a β1 or β2 Na⁺ channel subunit. Biologically active portions of a mNaᵥ1.3 α subunit protein can be used as targets for developing agents which modulate a Na⁺ channel mediated activity.

In one embodiment, a biologically active portion of a mNaᵥ1.3 α subunit protein comprises at least one transmembrane domain. Biologically active portions of mNaᵥ1.3 α subunit proteins mediate a mnaᵥ1.3 α subunit activity and include one or more features of a mNaᵥ1.3 α subunit protein. Biologically active portions, in which other regions of the protein are deleted, can be prepared by recombinant techniques and evaluated for one or more of the functional activities of a native mNaᵥ1.3 α subunit protein.

In one embodiment, the mNaᵥ1.3 α subunit protein has an amino acid sequence shown in SEQ ID NO:2, or is substantially homologous to SEQ ID NO:2, and retains the functional activity of the protein of SEQ ID NO:2, yet differs in amino acid sequence due to natural allelic variation or mutagenesis, as described in detail in the section on nucleotides.

Accordingly, in another embodiment, the mNaᵥ1.3 α subunit protein is a protein which comprises an amino acid sequence at least about 50%, 75%, 85%, 95%, 99% or more homologous to SEQ ID NO:2.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). The length of a reference sequence aligned for comparison purposes can be at least 50%, even 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

The comparison of sequences and determination of percent homology between two sequences can be accomplished using a mathematical algorithm. A, non-limiting example of a mathematical algorithm utilized for the comparison of sequences is the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength=12 to obtain nucleotide sequences homologous to mNaᵥ1.3 α subunit nucleic acid molecules of the invention. BLAST protein searches can be performed with the XBLAST program, score=50, wordlength=3 to obtain amino acid sequences homologous to mNaᵥ1.3 α subunit protein molecules of the invention. To obtain gapped alignments for comparison purposes, Gapped BLAST can be utilized as described in Altschul et al., Nucleic Acids Res. 25(17): 3389-3402, 1997. When utilizing BLAST and Gapped BLAST programs, the default parameters of the respective programs (e.g., XBLAST and NBLAST) can be used. See the website for the National Center for Biotechnology Information. Another non-limiting example of a mathematical algorithm utilized for the comparison of sequences is the algorithm of Myers and Miller, CABlOS (1989). Such an algorithm is incorporated into the ALIGN program (version 2.0) which is part of the GCG sequence alignment software package. When utilizing the ALIGN program for comparing amino acid sequences, a PAMI120 weight residue table, a gap length penalty of 12, and a gap penalty of 4 can be used.

The invention also provides mNaᵥ1.3 α subunit chimeric or fusion proteins. As used herein, a mnaᵥ1.3 α subunit “chimeric protein” or “fusion protein” comprises a mNaᵥ1.3 α subunit polypeptide operatively linked to a non-mNaᵥ1.3 α subunit polypeptide. A “mNaᵥ1.3 α subunit polypeptide” refers to a polypeptide having an amino acid sequence corresponding to mNaᵥ1.3 α subunit, whereas a “non-mNaᵥ1.3 α subunit polypeptide” refers to a polypeptide having an amino acid sequence corresponding to a protein which is not substantially homologous to the mNaᵥ1.3 α subunit protein, e.g., a protein which is different from the mNaᵥ1.3 α subunit protein and which is derived from the same or a different organism, or a protein which does not contain one or more of the features of the mNaᵥ1.3 α subunit proteins described herein. Within a mNaᵥ1.3 α subunit fusion protein the mNaᵥ1.3 α subunit polypeptide can correspond to all or a portion of a mNaᵥ1.3 α subunit protein. In a specific embodiment, a mNaᵥ1.3 α subunit fusion protein comprises at least one biologically active portion of a mNaᵥ1.3 α subunit protein. In another specific embodiment, a mNaᵥ1.3 α subunit fusion protein comprises at least two biologically active portions of a mNaᵥ1.3 α subunit protein. Within the fusion protein, the term “operatively linked” is intended to indicate that the mNaᵥ1.3 α subunit polypeptide and the non-mNaᵥ1.3 α subunit polypeptide are fused in-frame to each other. The non-mNaᵥ1.3 α subunit polypeptide can be fused to the N-terminus or C-terminus of the mNaᵥ1.3 α subunit polypeptide.

For example, in one embodiment, the fusion protein is a GST-mNaᵥ1.3 α subunit fusion protein in which the mNaᵥ1.3 α subunit sequences are fused to the C-terminus of the GST sequences. Such fusion proteins can facilitate the purification of recombinant mNaᵥ1.3 α subunit.

In another embodiment, the fusion protein is a mNaᵥ1.3 α subunit protein containing a heterologous signal sequence at its N-terminus. In certain host cells (e.g., mammalian host cells), expression of mNaᵥ1.3 α subunit can be increased through use of a heterologous signal sequence.

The mNaᵥ1.3 α subunit fusion proteins of the invention can be incorporated into pharmaceutical compositions and administered to a subject in vivo. The mNaᵥ1.3 α subunit fusion proteins can be used to affect the bioavailability of a mNaᵥ1.3 α subunit substrate. Use of mNaᵥ1.3 α subunit fusion proteins may be useful therapeutically for the treatment of disorders related to Na⁺ channel activity, e.g., neuropathic pain.

Moreover, the mNaᵥ1.3 α subunit-fusion proteins of the invention can be used as immunogens to produce anti-mNaᵥ1.3 α subunit antibodies in a subject, to purify mNaᵥ1.3 or subunit ligands and in screening assays to identify molecules which inhibit the interaction of mNaᵥ1.3 α subunit with a mNaᵥ1.3 α subunit substrate.

A mNaᵥ1.3 α subunit chimeric or fusion protein of the invention can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. The fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A mNaᵥ1.3 α subunit-encoding nucleic acid can be cloned into such an expression vector such that the fusion moiety is linked in-frame to the mNaᵥ1.3 α subunit protein.

The present invention also pertains to variants of the mNaᵥ1.3 α subunit proteins that function as either mNaᵥ1.3 α subunit agonists (mimetics) or as mNaᵥ1.3 α subunit antagonists. Variants of the mNaᵥ1.3 α subunit proteins can be generated by mutagenesis, e.g., discrete point mutation or truncation of a mNaᵥ1.3 α subunit protein. An agonist of the mNaᵥ1.3 α subunit proteins can retain substantially the same, or a subset, of the biological activities of the naturally occurring form of a mNaᵥ1.3 α subunit protein. An antagonist of a mNaᵥ1.3 α subunit protein can inhibit one or more of the activities of the naturally occurring form of the mNaᵥ1.3 α subunit protein by, for example, competitively modulating a Na⁺ channel mediated activity of a mNaᵥ1.3 α subunit protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the mNaᵥ1.3 α subunit protein.

In one embodiment, variants of a mNaᵥ1.3 α subunit protein which function as either mNaᵥ1.3 α subunit agonists (mimetics) or as mNaᵥ1.3 α subunit antagonists can be identified by screening combinatorial libraries of mutants, e.g., truncation mutants, of a mNaᵥ1.3 α subunit protein for mNaᵥ1.3 α subunit protein agonist or antagonist activity. In one embodiment, a variegated library of mNaᵥ1.3 α subunit variants is generated by combinatorial mutagenesis at the nucleic acid level and is encoded by a variegated gene library. A variegated library of mNaᵥ1.3 α subunit variants can be produced by, for example, enzymatically ligating a mixture of synthetic oligonucleotides into gene sequences such that a degenerate set of potential mNaᵥ1.3 α subunit sequences is expressible as individual polypeptides, or alternatively, as a set of larger fusion proteins (e.g., for phage display) containing the set of mNaᵥ1.3 α subunit sequences therein. There are a variety of methods that can be used to produce libraries of potential mNaᵥ1.3 α subunit variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential mNaᵥ1.3 α subunit sequences. Methods for synthesizing degenerate oligonucleotides are known in the art (see, e.g., Narang, S. A. Tetrahedron 39:3, 1983; Itakura et al. Annu. Rev. Biochem. 53:323, 1984; Itakura et al. Science 198:1056, 1984; Ike et al. Nucleic Acid Res. 11:477, 1983.

In addition, libraries of fragments of a mNaᵥ1.3 α subunit protein coding sequence can be used to generate a variegated population of mNaᵥ1.3 α subunit fragments for screening and subsequent selection of variants of a mNaᵥ1.3 α subunit protein.

The mNaᵥ1.3 α subunit proteins described herein can be directly or indirectly labeled with a detectable substance to facilitate detection of the bound or unbound binding agent. Suitable detectable substances include various enzymes, prosthetic groups, fluorescent materials, luminescent materials and radioactive materials.

An isolated mNaᵥ1.3 α subunit protein, or a portion or fragment thereof, can be used as an immunogen to generate antibodies that bind mNaᵥ1.3 α subunit using standard techniques for polyclonal and monoclonal antibody preparation. A full-length mNaᵥ1.3 α subunit protein can be used or, alternatively, the invention provides antigenic peptide fragments of mNaᵥ1.3 α subunit for use as immunogens. The antigenic peptide of mNaᵥ1.3 α subunit comprises the amino acid sequence of SEQ ID NO:2, or at least 8 amino acid residues of the amino acid sequence of SEQ ID NO:2, and encompasses an epitope of mNaᵥ1.3 α subunit such that an antibody raised against the peptide forms a specific immune complex with mNaᵥ1.3 α subunit. Preferably, the antigenic peptide comprises at least 10, 15, 20 or 30 amino acid residues.

A mNaᵥ1.3 α subunit immunogen typically is used to prepare antibodies by immunizing a suitable subject, (e.g., rabbit, goat, mouse or other mammal) with the immunogen. An appropriate immunogenic preparation can contain, for example, recombinantly expressed mNaᵥ1.3 α subunit protein or a chemically synthesized mNaᵥ1.3 α subunit polypeptide. The preparation can further include an adjuvant, such as Freund's complete or incomplete adjuvant, or similar immunostimulatory agent. Immunization of a suitable subject with an immunogenic mNaᵥ1.3 α subunit preparation induces a polyclonal anti-mNaᵥ1.3 α subunit antibody response.

Accordingly, another aspect of the invention pertains to anti-mNaᵥ1.3 α subunit antibodies. The term “antibody” as used herein refers to immunoglobulin molecules and immunologically active portions of immunoglobulin molecules, i.e., molecules that contain an antigen binding site which specifically binds (immunoreacts with) an antigen, such as mNaᵥ1.3 α subunit. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind mNaᵥ1.3 α subunit. The term “monoclonal antibody” or “monoclonal antibody composition”, as used herein, refers to a population of antibody molecules that contain only one species of an antigen binding site capable of immunoreacting with a particular epitope of mNaᵥ1.3 α subunit. A monoclonal antibody composition thus typically displays a single binding affinity for a particular mNaᵥ1.3 α subunit protein with which it immunoreacts.

Polyclonal anti-mNaᵥ1.3 α subunit antibodies can be prepared as described above by immunizing a suitable subject with a mNaᵥ1.3 α subunit immunogen. The anti-mNaᵥ1.3 α subunit antibody titer in the immunized subject can be monitored over time by standard techniques, such as with an enzyme linked immunosorbent assay (ELISA) using immobilized mNaᵥ1.3 α subunit. If desired, the antibody molecules directed against mNaᵥ1.3 α subunit can be isolated from the mammal (e.g., from the blood) and further purified by known techniques, such as protein A chromatography to obtain the IgG fraction. At an appropriate time after immunization, e.g., when the anti-mNaᵥ1.3 α subunit antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. J. Immunol. 127:539-46, 1981; Brown et al. J. Biol. Chem. 255:4980-83, 1980; Yeh et al. Proc. Natl. Acad. Sci. USA 76:2927-31, 1976; and Yeh et al. Int. J. Cancer 29:269-75, 1982), the more recent human B cell hybridoma technique (Kozbor et al. Immunol Today 4:72, 1983), the EBV-hybridoma technique (Cole et al. Monoclonal Antibodies and Cancer Therapy, Alan R. Liss, Inc., pp. 77-96, 1985) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner Yale J. Biol. Med., 54:387-402, 1981; M. L. Gefter et al. Somatic Cell Genet. 3:231-36, 1977). Briefly, an immortal cell line (typically a myeloma) is fused to lymphocytes (typically splenocytes) from a mammal immunized with a mNaᵥ1.3 α subunit immunogen as described above, and the culture supernatants of the resulting hybridoma cells are screened to identify a hybridoma producing a monoclonal antibody that binds mNaᵥ1.3 α subunit.

Any of the many well known protocols used for fusing lymphocytes and immortalized cell lines can be applied for the purpose of generating an anti-mNaᵥ1.3 α subunit monoclonal antibody (see, e.g., G. Galfre et al. Nature 266:55052, 1977; Gefter et al. Somatic Cell Genet., cited supra; Lerner, Yale J. Biol. Med., cited supra; Kenneth, Monoclonal Antibodies, cited supra). Moreover, the skilled worker will appreciate that there are many variations of such methods which also would be useful.

Alternative to preparing monoclonal antibody-secreting hybridomas, a monoclonal anti-mNaᵥ1.3 α subunit antibody can be identified and isolated by screening a recombinant combinatorial immunoglobulin library (e.g., an antibody phage display library) with mNaᵥ1.3 α subunit to thereby isolate immunoglobulin library members that bind mNaᵥ1.3 α subunit. Kits for generating and screening phage display libraries are commercially available (e.g., the Pharmacia Recombinant Phage Antibody System, Catalog No. 27-9400-01). Additionally, examples of methods and reagents particularly amenable for use in generating and screening antibody display libraries can be found in, for example, Ladner et al. U.S. Pat. No. 5,223,409; Kang et al. PCT International Publication No. WO 92/18619; and McCafferty et al. Nature 348:552-554, 1990.

Additionally, recombinant anti-mNaᵥ1.3 α subunit antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. Science 240:1041-1043, 1988; Liu et al. Proc. Natl. Acad. Sci. USA 84:3439-3443, 1987; Liu et al. J. Immunol. 139:3521-3526, 1987; Sun et al. Proc. Natl. Acad. Sci. USA 84:214-218, 1987; Nishimura et al. Canc. Res. 47:999-1005, 1987; Wood et al. Nature 314:446-449, 1985; and Shaw et al. J. Natl. Cancer Inst. 80:1553-1559, 1988); Morrison, S. L. Science 229:1202-1207, 1985; Oi et al. BioTechniques 4:214, 1986; Winter U.S. Pat. No. 5,225,539; Jones et al. Nature 321:552-525, 1986; Verhoeyan et al. Science 239:1534, 1988; and Beidler et al. J. Immunol. 141:4053-4060, 1988.

An anti-mNaᵥ1.3 α subunit antibody (e.g., monoclonal antibody) can be used to isolate mNaᵥ1.3 α subunit by standard techniques, such as affinity chromatography or immunoprecipitation. An anti-mNaᵥ1.3 α subunit antibody can facilitate the purification of natural mNaᵥ1.3 α subunit from cells and of recombinantly produced mNaᵥ1.3 α subunit expressed in host cells. Moreover, an anti-mNaᵥ1.3 α subunit antibody can be used to detect mNaᵥ1.3 α subunit protein (e.g., in a cellular lysate or cell supernatant) in order to evaluate the abundance and pattern of expression of the mNaᵥ1.3 α subunit protein. Anti-mNaᵥ1.3 α subunit antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ³⁵ S, or ³H.

### Recombinant Expression Vectors and Host Cells

Another aspect of the invention pertains to vectors, preferably expression vectors, containing a nucleic acid encoding a mNaᵥ1.3 α subunit protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other types of vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. Herein, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., mNaᵥ1.3 α subunit proteins, mutant forms of mNaᵥ1.3 α subunit proteins, fusion proteins, and the like).

The recombinant expression vectors of the invention can be designed for expression of mNaᵥ1.3 α subunit proteins in prokaryotic or eukaryotic cells. For example, mNaᵥ1.3 α subunit proteins can be expressed in bacterial cells such as E. coli, insect cells (using baculovirus expression vectors), yeast cells, amphibian cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins.

Purified fusion proteins can be utilized in mNaᵥ1.3 α subunit activity assays, (e.g., direct assays or competitive assays described in detail below), or to generate antibodies specific for mNaᵥ1.3 α subunit, for example.

In another embodiment, the mNaᵥ1.3 α subunit expression vector is a yeast expression vector. Examples of vectors for expression in yeast S. cerivisae include pYepSec1 (Baldari, et al., (1987) EMBO J. 6:229-234), pMFa (Kurjan and Herskowitz, Cell 30:933-943, 1982), pJRY88 (Schultz et al., Gene 54:113-123, 1987), pYES2 (Invitrogen Corporation, San Diego, Calif.), and picZ (InVitrogen Corp, San Diego, Calif.).

Alternatively, mNaᵥ1.3 α subunit proteins can be expressed in insect cells using baculovirus expression vectors. Baculovirus vectors available for expression of proteins in cultured insect cells (e.g., Sf 9 cells) include the pAc series (Smith et al. Mol. Cell. Biol. 3:2156-2165, 1983) and the pVL series (Lucklow and Summers Virol. 170:31-39, 1989).

In yet another embodiment, a nucleic acid of the invention is expressed in mammalian cells using a mammalian expression vector. Examples of mammalian expression vectors include pCDM8 (Seed, B. Nature 329:840, 1987) and pMT2PC (Kaufman et al. EMBO J. 6:187-195, 1987). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. Genes Dev. 1:268-277, 1987), lymphoid-specific promoters (Calame and Eaton Adv. Immunol. 43:235-275, 1988), in particular promoters of T cell receptors (Winoto and Baltimore EMBO J. 8:729-733, 1989) and immunoglobulins (Banerji et al. Cell 33:729-740, 1983; Queen and Baltimore Cell 33:741-748, 1983), neuron-specific promoters (e.g., the neurofilament promoter; Byrne and Ruddle Proc. Natl. Acad. Sci. USA 86:5473-5477, 1989), pancreas-specific promoters (Edlund et al. Science 230:912-916, 1985), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss Science 249:374-379, 1990 and the α-fetoprotein promoter (Campes and Tilghman Genes Dev. 3:537-546, 1989).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to mNaᵥ1.3 α subunit mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Trends in Genet., Vol. 1(1) 1986.

Another aspect of the invention pertains to host cells into which a nucleic acid, e.g., a mNaᵥ1.3 α subunit mRNA, or a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a mNaᵥ1.3 α subunit protein can be expressed in bacterial cells such as E. coli, insect cells, yeast cells, Xenopus cells, e.g., Xenopus oocytes, or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed, Cold Spring Harbor Laboratory, Cold Spring Harbor Laboratory Press, Cold Spring Harbor, N.Y., 1989), and other laboratory manuals. Nucleic acids can also be introduced by microinjection.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Selectable markers include those which confer resistance to drugs, such as G418, hygromycin and methotrexate. Nucleic acid encoding a selectable marker can be introduced into a host cell on the same vector as that encoding a mNaᵥ1.3 α subunit protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) a mNaᵥ1.3 α subunit protein. Accordingly, the invention further provides methods for producing a mNaᵥ1.3 α subunit protein using the host cells of the invention.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

### Uses and Methods of the Invention

The nucleic acid molecules, proteins, protein homologues, and antibodies described herein can be used in one or more of the following methods: a) screening assays; b) predictive medicine (e.g., diagnostic assays, prognostic assays, monitoring clinical trials, and pharmacogenetics); and c) methods of treatment (e.g., therapeutic and prophylactic).

The isolated nucleic acid molecules of the invention can be used, for example, to express mNaᵥ1.3 α subunit protein (e.g., via a recombinant expression vector in a host cell in gene therapy applications), to detect mNaᵥ1.3 α subunit mRNA (e.g., in a biological sample) or a genetic alteration in a gene encoding a mNaᵥ1.3 α subunit protein, and to modulate mNaᵥ1.3 ac subunit activity, as described further below. The mNaᵥ1.3 α subunit proteins can be used to treat disorders characterized by insufficient or excessive production of a mNaᵥ1.3 α subunit substrate or production of mNaᵥ1.3 α subunit inhibitors. In addition, the mNaᵥ1.3 α subunit proteins can be used to screen for naturally occurring mNaᵥ1.3 α subunit substrates, to screen for drugs or compounds which modulate mNaᵥ1.3 α subunit activity, as well as to treat disorders characterized by insufficient or excessive production of mNaᵥ1.3 α subunit protein or production of mNaᵥ1.3 α subunit protein forms which have decreased or aberrant activity compared to mNaᵥ1.3 α subunit wild type protein. Moreover, the anti-mNaᵥ1.3 α subunit antibodies of the invention can be used to detect and isolate mNaᵥ1.3 α subunit proteins and modulate mNaᵥ1.3 α subunit activity.

### Screening Assays

The invention provides methods for identifying modulators, i.e., candidate or test compounds or agents (e.g., proteins, peptides, peptidomimetics, peptoids, small molecules or other drugs) which bind to Na⁺ channels comprising a mNaᵥ1.3 α subunit described herein. Compounds thus identified can be used to modulate the activity of these Na⁺ channels e.g., in a therapeutic protocol.

In one embodiment, the invention provides assays for screening test compounds which are substrates of Na⁺ channels that include a mNaᵥ1.3 α subunit described herein, or a biologically active portion of the subunit. In another embodiment, the invention provides assays for screening candidate or test compounds that bind to or modulate an activity of these Na⁺ channels.

Ion channel-modulating compounds can be identified through both in vitro (e.g., cell and non-cell based) and in vivo methods. In one embodiment, ion influx assays are used to measure Na⁺ channel activity.

Assays to measure ion channel activity include flux assays, patch-clamp electrophysiology, and two electrode voltage clamp electrophysiology (see, e.g., Lin et al., Neuron 18:153-166, 1997). Patch-clamp physiology can be performed as follows. Briefly, a pipette tip containing a small electrode is pressed against a cell membrane to create a tight seal between the pipette and the membrane. The electrode captures the ions flowing through the membrane defined by the edges of the pipette tip. Various configurations can be employed to measure currents within the cell or within a patch of membrane or over the entire cell.

Two electrode voltage-clamp (TEVC) physiology can be performed as follows. Briefly, two sharp microelectrodes are pressed through a cell membrane. One electrode monitors membrane potential and the other electrode injects current to hold the membrane potential at the desired level. Both patch-clamp and TEVC techniques provide information regarding both kinetics and intensity of ion channel currents.

High-throughput electrophysiology can be performed, e.g., as described in U.S. Pat. No. 6,268,168 and U.S. Pat. No. 6,048,722, the contents of which are incorporated herein by reference.

Assays that measure changes in ion concentration can be used. For example, the test system can be loaded with detectable Na⁺ (e.g., radiolabeled Na⁺). Detection of the Na⁺ can give an indication of a change in Na⁺ concentration. In one embodiment, the assay involves detection of Na⁺ following stimulation by application of a voltage to the test system (e.g., a cell or an enclosed membrane preparation). Sodium-sensitive dyes, such as sodium green or corona red, may also be used to measure changes in ion concentration.

In one embodiment, Na⁺ channel modulation is assayed using a Xenopus oocyte system. For a detailed description of transient expression of ion channels and recording from Xenopus oocytes, see, e.g., Xu and Lipscombe, J. Neurosci. 21(16):5944-5951, 2001; Lin et al., supra). In another embodiment, the assay is a mammalian-cell based assay, e.g., using a human or mouse cell. A particular Na⁺ channel, such as a mNaᵥ1.3 α subunit channel, can be studied in isolation by transfection into a cell type that does not express other Na⁺ channels.

### Compounds

The test compounds of the present invention can be obtained singly or using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2678-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

Chemical compounds to be used as test compounds (i.e., potential inhibitor, antagonist, agonist) can be obtained from commercial sources or can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In one aspect the compounds are organic small molecules, that is, compounds having molecular weight less than 1,000 amu, alternatively between 350-750 amu. In other aspects, the compounds are: (i) those that are non-peptidic; (ii) those having between 1 and 5, inclusive, heterocyclyl, or heteroaryl ring groups, which may bear further substituents; (iii) those in their respective pharmaceutically acceptable salt forms; or (iv) those that are peptidic.

The term “heterocyclyl” refers to a nonaromatic 3-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring can be substituted by a substituent.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring can be substituted by a substituent.

The term “substituents” refers to a group “substituted” on an alkyl, cycloalkyl, aryl, heterocyclyl, or heteroaryl group at any atom of that group. Suitable substituents include, without limitation, alkyl, alkenyl, alkynyl, alkoxy, halo, hydroxy, cyano, nitro, amino, SO₃H, perfluoroalkyl, perfluoroalkoxy, methylenedioxy, ethylenedioxy, carboxyl, oxo, thioxo, imino(alkyl, aryl, aralkyl), S(O)ₙalkyl (where n is 0-2), S(O)ₙ aryl (where n is 0-2), S(O), heteroaryl (where n is 0-2), S(O), heterocyclyl (where n is 0-2), amine(mono-, di-, alkyl, cycloalkyl, aralkyl, heteroaralkyl, and combinations thereof), ester(alkyl, aralkyl, heteroaralkyl), amide(mono-, di-, alkyl, aralkyl, heteroaralkyl, and combinations thereof), sulfonamide(mono-, di-, alkyl, aralkyl, heteroaralkyl, and combinations thereof), unsubstituted aryl, unsubstituted heteroaryl, unsubstituted heterocyclyl, and unsubstituted cycloalkyl. In one aspect, the substituents on a group are independently any one single, or any subset of the aforementioned substituents.

Combinations of substituents and variables in compounds envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., transport, storage, assaying, therapeutic administration to a subject).

Pharmaceutically acceptable salts of the compounds herein include those derived from pharmaceutically acceptable inorganic and organic acids and bases. Examples of suitable acid salts include acetate, adipate, alginate, aspartate, benzoate, benzenesulfonate, bisulfate, butyrate, citrate, digluconate, ethanesulfonate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, pectinate, persulfatephosphate, picrate, pivalate, propionate, salicylate, succinate, sulfate, tartrate, thiocyanate, tosylate and undecanoate.

The compounds described herein can contain one or more asymmetric centers and thus occur as racemates and racemic mixtures, single enantiomers, individual diastereomers and diastereomeric mixtures. All such isomeric forms of these compounds are expressly included in the present invention. The compounds described herein can also be represented in multiple tautomeric forms, all of which are included herein. The compounds can also occur in cis- or trans- or E- or Z-double bond isomeric forms. All such isomeric forms of such compounds are expressly included in the present invention.

### Binding Assays

The ability of the test compound to bind to a Na⁺ channel comprising a mNaᵥ1.3 α subunit can also be evaluated. While Na⁺ channel binding is not a prerequisite for channel modulatory activity, compounds that bind a Na⁺ channel can be useful in modulating activity of the channel. This can be accomplished, for example, by coupling the compound, e.g., the substrate, with a radioisotope or enzymatic label such that binding of the compound, e.g., the substrate, to Na⁺ channels can be determined by detecting the labeled compound, e.g., substrate, in a complex. Alternatively, a Na⁺ channel comprising a mNaᵥ1.3 α subunit described herein could be coupled with a radioisotope or enzymatic label to monitor the ability of a test compound to modulate the complex. For example, compounds can be labeled with ¹²⁵I, ³⁵S, ¹⁴C, or ³H, either directly or indirectly, and the radioisotope detected by direct counting of radioemmission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a test compound to interact with a Na⁺ channel comprising a mNaᵥ1.3 α subunit or without the labeling of any of the interactants can be evaluated. For example, a microphysiometer can be used to detect the interaction of a compound with a Na⁺ channel without the labeling of either the compound or the Na⁺ channel (McConnell, H. M. et al., Science 257:1906-1912, 1992). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and a Na⁺ channel.

In yet another embodiment, a cell-free assay is provided in which a Na⁺ channel described herein or biologically active portion thereof is contacted with a test compound and the ability of the test compound to bind to the channel or biologically active portion thereof is evaluated. Preferably, the cell-free assay comprises a membrane. Cell-free assays involve preparing a reaction mixture of the target gene protein and the test compound under conditions and for a time sufficient to allow the two components to interact and bind, thus forming a complex that can be removed and/or detected.

The interaction between two molecules can also be detected, e.g., using fluorescence energy transfer (FET) (see, for example, Lakowicz et al., U.S. Pat. No. 5,631,169; Stavrianopoulos et al., U.S. Pat. No. 4,868,103). A fluorophore label on the first, ‘donor’ molecule is selected such that its emitted fluorescent energy will be absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determining the ability of a test compound to bind to a Na⁺ channel described herein can be accomplished using real-time Biomolecular Interaction Analysis (BIA) (see, e.g., Sjolander, S. and Urbaniczky, C., Anal. Chem. 63:2338-2345, 1991; and Szabo et al., Curr. Opin. Struct. Biol. 5:699-705, 1995). “Surface plasmon resonance” or “BIA” detects biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal that can be used as an indication of real-time reactions between biological molecules.

In one embodiment, the sample comprising the Na⁺ channel or the test compound is anchored onto a solid phase. The channel/test compound complexes anchored on the solid phase can be detected at the end of the reaction.

It may be desirable to immobilize either the Na⁺ channel, an anti-Na⁺ channel antibody or its target molecule to facilitate separation of complexed from uncomplexed forms of one or both of the proteins, as well as to accommodate automation of the assay. Binding of a test compound to a Na⁺ channel, or interaction of a Na⁺ channel with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided which adds a domain that allows one or both of the proteins to be bound to a matrix. For example, glutathione-S-transferase/mNaᵥ1.3 α subunit fusion proteins or glutathione-S-transferase/target fusion proteins can be adsorbed onto glutathione sepharose beads (Sigma Chemical, St. Louis, Mo.) or glutathione derivatized microtiter plates, which are then combined with the test compound or the test compound and a sample comprising the Na⁺ channel comprising the GST-tagged subunit, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above.

Other techniques for immobilizing a complex of Na⁺ channel subunits on matrices include using conjugation of biotin and streptavidin. For example, biotinylated mNaᵥ1.3 α subunit proteins can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical).

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In one embodiment, this assay is performed utilizing antibodies reactive with an epitope on the Na⁺ channel but which do not interfere with binding of the channel to a target molecule. Such antibodies can be derivatized to the wells of the plate, and unbound target or Na⁺ channels trapped in the wells by antibody conjugation. Methods for detecting such complexes, in addition to those described above for the GST-immobilized complexes, include immunodetection of complexes using antibodies reactive with a component of the Na⁺ channel, as well as enzyme-linked assays which rely on detecting an enzymatic activity associated with the channel.

Alternatively, cell free assays can be conducted in a liquid phase. In such an assay, the reaction products are separated from unreacted components, by any of a number of standard techniques, including but not limited to: differential centrifugation (see, for example, Rivas, G., and Minton, A. P., Trends Biochem Sci 18:284-7, 1993); chromatography (gel filtration chromatography, ion-exchange chromatography); electrophoresis and immunoprecipitation (see, for example, Ausubel, F. et al., eds. (1999) Current Protocols in Molecular Biology, J. Wiley: New York). Such resins and chromatographic techniques are known to one skilled in the art (see, e.g., Heegaard, N. H., J Mol Recognit 11:141-8, 1998; Hage, D. S., and Tweed, S. A., J Chromatogr B Biomed Sci Appl. 699:499-525, 1997). Further, fluorescence energy transfer may also be conveniently utilized, as described herein, to detect binding without further purification of the complex from solution. Preferably, cell free assays preserve the structure of the Na⁺ channel complex, e.g., by including a membrane component or synthetic membrane components.

In a specific embodiment, the assay includes contacting the Na⁺ channel or channel comprising biologically active portions of the mNaᵥ1.3 α subunit with a known compound which binds the channel to form an assay mixture, contacting the assay mixture with a test compound, and determining the ability of the test compound to interact with a Na⁺ channel, wherein determining the ability of the test compound to interact with a Na⁺ channel includes determining the ability of the test compound to preferentially bind to the Na⁺ channel, or to modulate the activity of the channel, as compared to the known compound.

The Na⁺ channels described herein can, in vivo, interact with one or more cellular or extracellular macromolecules, such as proteins. For the purposes of this discussion, such cellular and extracellular macromolecules are referred to herein as “binding partners.” Compounds that disrupt such interactions can be useful in regulating the activity of the target gene product. Such compounds can include, but are not limited to molecules such as antibodies, peptides, and small molecules.

To identify compounds that interfere with the interaction between the Na⁺ channel and an extracellular binding partner(s), a reaction mixture containing the target gene product and the binding partner is prepared, under conditions and for a time sufficient, to allow the two products to form a complex. In order to test an inhibitory agent, the reaction mixture is provided in the presence and absence of the test compound. The test compound can be initially included in the reaction mixture, or can be added at a time subsequent to the addition of the Na⁺ channel and its cellular or extracellular binding partner. Control reaction mixtures are incubated without the test compound or with a placebo. The formation of any complexes between the Na⁺ channel and the cellular or extracellular binding partner is then detected. The formation of a complex in the control reaction, but not in the reaction mixture containing the test compound, indicates that the compound interferes with the interaction of the target gene product and the interactive binding partner. Additionally, complex formation within reaction mixtures containing the test compound and normal target gene product can also be compared to complex formation within reaction mixtures containing the test compound and a Na⁺ channel comprising one or more mutant subunits. This comparison can be important in those cases wherein it is desirable to identify compounds that disrupt interactions of mutant but not normal target gene products.

These assays can be conducted in a heterogeneous or homogeneous format. Heterogeneous assays involve anchoring either the Na⁺ channel or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the target gene products and the binding partners, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the target gene product or the interactive cellular or extracellular binding partner is anchored onto a solid surface (e.g., a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct the assay, the partner of the immobilized species is exposed to the coated surface with or without the test compound. After the reaction is complete, unreacted components are removed (e.g., by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. For example, a preformed complex of the target gene product and the interactive cellular or extracellular binding partner product is prepared in that either the Na⁺ channel subunits or their binding partners are labeled, but the signal generated by the label is quenched due to complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-binding partner interaction can be identified.

In yet another aspect, the Na⁺ channel proteins or fragments thereof can be used as “bait proteins” in a two-hybrid assay or three-hybrid assay (see, e.g., U.S. Pat. No. 5,283,317; Zervos et al., Cell 72:223-232, 1993; Madura et al., J. Biol. Chem. 268:12046-12054, 1993; Bartel et al., Biotechniques 14:920-924, 1993; Iwabuchi et al., Oncogene 8:1693-1696, 1993; and Brent WO94/10300), to identify other proteins, which bind to or interact with Na⁺ channel proteins (“Na⁺ channel-binding proteins” or “Na⁺ channel-bp”) and are involved in Na⁺ channel activity. Such Na⁺ channel-bps can be activators or inhibitors of signals by the Na⁺ channels or Na⁺-sensitive targets.

The two-hybrid system is based on the modular nature of most transcription factors, which consist of separable DNA-binding and activation domains. Briefly, the assay utilizes two different DNA constructs. In one construct, the gene that codes for a Na⁺ channel α subunitprotein or fragment thereof (e.g., corresponding to a soluble portion of an extracellular domain of the subunit) is fused to a gene encoding the DNA binding domain of a known transcription factor (e.g., GAL-4). In the other construct, a DNA sequence, from a library of DNA sequences, which encodes an unidentified protein (“prey” or “sample”) is fused to a gene that codes for the activation domain of the known transcription factor. (Alternatively, the Na⁺ channel subunit can be the fused to the activator domain.) If the “bait” and the “prey” proteins are able to interact, in vivo, forming a Na⁺ channel subunit-dependent complex, the DNA-binding and activation domains of the transcription factor are brought into close proximity. This proximity allows transcription of a reporter gene (e.g., lacZ) that is operably linked to a transcriptional regulatory site responsive to the transcription factor. Expression of the reporter gene can be detected and cell colonies containing the functional transcription factor can be isolated and used to obtain the cloned gene that encodes the protein that interacts with the Na⁺ channel subunit.

In another embodiment, modulators of Na⁺ channel subunit expression are identified. For example, a cell or cell free mixture is contacted with a candidate compound and the expression of mNaᵥ1.3 α subunit mRNA or protein evaluated relative to the level of expression of mNaᵥ1.3 α subunit mRNA or protein in the absence of the candidate compound. When expression of mNaᵥ1.3 α subunit mRNA or protein is greater in the presence of the candidate compound than in its absence, the candidate compound is identified as a stimulator of mNaᵥ1.3 α subunit mRNA or protein expression. Alternatively, when expression of mNaᵥ1.3 α subunit mRNA or protein is less (statistically significantly less) in the presence of the candidate compound than in its absence, the candidate compound is identified as an inhibitor of mNaᵥ1.3 α subunit mRNA or protein expression. The level of mNaᵥ1.3 α subunit mRNA or protein expression can be determined by methods described herein for detecting mNaᵥ1.3 α subunit mRNA or protein.

In another aspect, the invention pertains to a combination of two or more of the assays described herein. For example, a modulating agent can be identified using a cell-based or a cell free assay, and the ability of the agent to modulate the activity of a Na⁺ channel can be confirmed in vivo, e.g., in an animal such as an animal model for a pain disorder or a disorder associated with stroke or traumatic brain injury.

This invention further pertains to novel agents identified by the above-described screening assays. Accordingly, it is within the scope of this invention to further use an agent identified as described herein (e.g., a mNaᵥ1.3 α subunit channel-modulating agent, an antisense nucleic acid molecule corresponding to one or more of the Na⁺ channel subunits described herein, a channel-specific antibody, or a mNaᵥ1.3 α subunit channel-binding partner) in an appropriate animal model to determine the efficacy, toxicity, side effects, or mechanism of action, of treatment with such an agent. Furthermore, novel agents identified by the above-described screening assays can be used for treatments as described herein.

Diagnostic and prognostic assays of the invention include methods for assessing the expression level of mNaᵥ1.3 α subunit and for identifying variations and mutations in the nucleotide or amino acid sequence of mNaᵥ1.3 α subunit molecules.

Expression Monitoring and Profiling. The presence, level, or absence of mNaᵥ1.3 α subunit protein or nucleic acid in a biological sample can be evaluated by obtaining a biological sample from a test subject and contacting the biological sample with a compound or an agent capable of detecting mNaᵥ1.3 α subunit protein or nucleic acid (e.g., mRNA, genomic DNA) that encodes mNaᵥ1.3 α subunit protein such that the presence of the protein or nucleic acid is detected in the biological sample. The term “biological sample” includes tissues, cells and biological fluids isolated from a subject, as well as tissues, cells and fluids present within a subject. A preferred biological sample is brain tissue. The level of expression of the mNaᵥ1.3 α subunit gene can be measured in a number of ways, including, but not limited to: measuring the mRNA encoded by the mNaᵥ1.3 α subunit gene; measuring the amount of protein encoded by the mNaᵥ1.3 α subunit gene; or measuring the activity of the protein encoded by the mNaᵥ1.3 α subunit.

The level of mRNA corresponding to a mNaᵥ1.3 α subunit gene in a cell can be determined both by in situ and by in vitro formats.

The isolated mRNA can be used in hybridization or amplification assays that include, but are not limited to, Southern or Northern analyses, polymerase chain reaction analyses and probe arrays. One diagnostic method for the detection of mRNA levels involves contacting the isolated mRNA with a nucleic acid molecule (probe) that can hybridize to the mRNA encoded by the gene being detected. The nucleic acid probe can be, for example, a full-length Na⁺ channel α subunitnucleic acid, such as the nucleic acids described herein, or a portion thereof, such as an oligonucleotide of at least 7, 15, 30, 50, 100, 250 or 500 nucleotides in length and sufficient to specifically hybridize under stringent conditions to Na⁺ channel α subunitmRNA or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the mNaᵥ1.3 α subunit genes.

The level of mRNA in a sample that is encoded by a mNaᵥ1.3 α subunit gene can be evaluated with nucleic acid amplification, e.g., by rtPCR (Mullis (1987) U.S. Pat. No. 4,683,202), ligase chain reaction (Barany, Proc. Natl. Acad. Sci. USA 88:189-193, 1991), self sustained sequence replication (Guatelli et al., Proc. Natl. Acad. Sci. USA 87:1874-1878, 1990), transcriptional amplification system (Kwoh et al., Proc. Natl. Acad. Sci. USA 86:1173-1177, 1989), Q-BetaReplicase (Lizardi et al., Bio/Technology 6:1197, 1988), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the mNaᵥ1.3 α subunit gene being analyzed.

In another embodiment, the methods herein include further contacting a control sample with a compound or agent capable of detecting mNaᵥ1.3 α subunit mRNA, or genomic DNA, and comparing the presence of mNaᵥ1.3 α subunit mRNA or genomic DNA in the control sample with the presence of mNaᵥ1.3 α subunit mRNA or genomic DNA in the test sample. In still another embodiment, serial analysis of gene expression, as described in U.S. Pat. No. 5,695,937, is used to detect mNaᵥ1.3 α subunit transcript levels.

A variety of methods can be used to determine the level of protein encoded by mNaᵥ1.3 α subunit genes. In general, these methods include contacting an agent that selectively binds to the protein, such as an antibody with a sample, to evaluate the level of protein in the sample. In a specific embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect mNaᵥ1.3 α subunit protein in a biological sample in vitro as well as in vivo. In vitro techniques for detection of proteins include enzyme linked immunosorbent assays (ELISAs), immunoprecipitations, immunofluorescence, enzyme immunoassay (EIA), radioimmunoassay (RIA), and Western blot analysis. In vivo techniques for detection of proteins include introducing into a subject a labeled anti-mNaᵥ1.3 α subunit antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-mNaᵥ1.3 α subunit antibody positioned on an antibody array (as described below). The sample can be detected, e.g., with avidin coupled to a fluorescent label.

In another embodiment, the methods further include contacting the control sample with a compound or agent capable of detecting a mNaᵥ1.3 α subunit protein, and comparing the presence of the protein in the control sample with the presence of the protein in the test sample.

The invention also includes kits for detecting the presence of mNaᵥ1.3 α subunit proteins in a biological sample. For example, the kit can include a compound or agent capable of detecting mNaᵥ1.3 α subunit protein or mRNA in a biological sample; and a standard. The compound or agent can be packaged in a suitable container. The kit can further comprise instructions for using the kit to detect mNaᵥ1.3 α subunit proteins or nucleic acids.

For antibody-based kits, the kit can include: (1) a first antibody (e.g., attached to a solid support) which binds to a polypeptide corresponding to a marker of the invention; and, optionally, (2) a second, different antibody which binds to either the polypeptide or the first antibody and is conjugated to a detectable agent.

For oligonucleotide-based kits, the kit can include: (1) an oligonucleotide, e.g., a detectably labeled oligonucleotide, which hybridizes to a nucleic acid sequence encoding a polypeptide corresponding to a marker of the invention or (2) a pair of primers useful for amplifying a nucleic acid molecule corresponding to a marker of the invention. The kit can also include a buffering agent, a preservative, or a protein stabilizing agent. The kit can also includes components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

The diagnostic methods described herein can identify subjects having, or at risk of developing, a disease or disorder associated with Na⁺ channel expression or activity. As used herein, the term “unwanted” includes an unwanted phenomenon involved in a biological response such as neuropathic pain.

In one embodiment, a disease or disorder associated with Na⁺ channel expression or activity is identified. A test sample is obtained from a subject and one or more Na⁺ channel proteins or nucleic acids (e.g., mRNA or genomic DNA) are evaluated, wherein the level, e.g., the presence or absence, of a Na⁺ channel protein or nucleic acid is diagnostic for a subject having or at risk of developing a disease or disorder associated with Na⁺ channel expression or activity. As used herein, a “test sample” refers to a biological sample obtained from a subject of interest, including a biological fluid (e.g., serum), cell sample, or tissue, e.g., brain tissue.

The prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with mNaᵥ1.3 α subunit expression or activity. For example, such methods can be used to determine whether a subject can be effectively treated with an agent for a pain disorder or for traumatic brain injury.

In another aspect, the invention features a computer medium having a plurality of digitally encoded data records. Each data record includes a value representing the level of expression or activity of a mNaᵥ1.3 α subunit channel in a sample, and a descriptor of the sample. The descriptor of the sample can be an identifier of the sample, a compound which with the sample was treated, a subject from which the sample was derived (e.g., a patient), a diagnosis, or a treatment (e.g., a preferred treatment). In a specific embodiment, the data record further includes values representing the level of expression of genes other than the mNaᵥ1.3 α subunit channel (e.g., other genes associated with a disorder related to activity of the mNaᵥ1.3 α subunit channel, or other genes on an array). The data record can be structured as a table, e.g., a table that is part of a database such as a relational database (e.g., a SQL database of the Oracle or Sybase database environments).

Also featured is a method of evaluating a sample. The method includes providing a sample, e.g., from the subject, and determining a gene expression profile of the sample, wherein the profile includes a value representing the level of mNaᵥ1.3 α subunit channel expression or activity. The method can further include comparing the value or the profile (i.e., multiple values) to a reference value or reference profile. The gene expression profile of the sample can be obtained by any of the methods described herein (e.g., by providing a nucleic acid from the sample and contacting the nucleic acid to an array, or by assaying the activity of a mNaᵥ1.3 α subunit channel in the sample). The method can be used to diagnose a disorder in a subject wherein a change in mNaᵥ1.3 α subunit expression is an indication that the subject has or is disposed to having a disorder. The method can be used to monitor a treatment, e.g., for pain in a subject. For example, the gene expression profile can be determined for a sample from a subject undergoing treatment. The profile can be compared to a reference profile or to a profile obtained from the subject prior to treatment or prior to onset of the disorder (see, e.g., Golub et al., Science 286:531, 1999).

In yet another aspect, the invention features a method of evaluating a test compound (see also, “Screening Assays”, above). The method includes providing a cell and a test compound; contacting the test compound to the cell; obtaining a subject expression profile for the contacted cell; and comparing the subject expression profile to one or more reference profiles. The profiles include a value representing the level of mNaᵥ1.3 α subunit activity or expression. In a specific embodiment, the subject activity or expression profile is compared to a target profile, e.g., a profile for a normal cell or for a desired condition of a cell. The test compound is evaluated favorably if the subject expression profile is more similar to the target profile than an expression profile obtained from an uncontacted cell.

In another aspect, the invention features, a method of evaluating a subject. The method includes: a) obtaining a sample from a subject, e.g., from a caregiver, e.g., a caregiver who obtains the sample from the subject; b) determining a subject expression profile for the sample. Optionally, the method further includes either or both of steps:

c) comparing the subject expression profile to one or more reference expression profiles; and d) selecting the reference profile most similar to the subject reference profile. The subject expression profile and the reference profiles include a value representing the level of mNaᵥ1.3 α subunit activity or expression. A variety of routine statistical measures can be used to compare two reference profiles. One possible metric is the length of the distance vector that is the difference between the two profiles. Each of the subject and reference profile is represented as a multi-dimensional vector, wherein each dimension is a value in the profile.

The method can further include transmitting a result to a caregiver. The result can be the subject expression profile, a result of a comparison of the subject expression profile with another profile, a most similar reference profile, or a descriptor of any of the aforementioned. The result can be transmitted across a computer network, e.g., the result can be in the form of a computer transmission, e.g., a computer data signal embedded in a carrier wave.

Also featured is a computer medium having executable code for effecting the following steps: receive a subject expression profile (e.g., any subject expression profile described herein); access a database of reference expression profiles; and either i) select a matching reference profile most similar to the subject expression profile or ii) determine at least one comparison score for the similarity of the subject expression profile to at least one reference profile. The subject expression profile, and the reference expression profiles each include a value representing the level of mNaᵥ1.3 α subunit activity or expression.

### Arrays and Uses Thereof

In another aspect, the invention features an array that includes a substrate having a plurality of addresses. At least one address of the plurality includes a capture probe that binds specifically to a molecule corresponding to a Na⁺ channel α subunit, e.g., a mNaᵥ1.3 α subunit nucleic acid or polypeptide. The array can have a density of at least 10, 100, 1,000, or 10,000 or more addresses/cm², and ranges between. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In a specific embodiment, at least one address of the plurality includes a nucleic acid capture probe that hybridizes specifically to a mNaᵥ1.3 α subunit nucleic acid, e.g., the sense or anti-sense strand. A subset of addresses of the plurality of addresses can be a nucleic acid capture probe for a Na⁺ channel gene encoding a mNaᵥ1.3 α subunit. Each address of the subset can include a capture probe that hybridizes to a different region of a mNaᵥ1.3 α subunit nucleic acid. The array can be used to sequence the gene by hybridization (see, e.g., U.S. Pat. No. 5,695,940).

An array can be generated by various methods, e.g., by photolithographic methods (see, e.g., U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another embodiment, at least one address of the plurality includes a polypeptide capture probe that binds specifically to a mNaᵥ1.3 α subunit polypeptide or fragment thereof. The polypeptide can be a naturally-occurring interaction partner of a mNaᵥ1.3 α subunit polypeptide. Preferably, the polypeptide is an antibody, e.g., an antibody described herein (see “Anti-mNaᵥ1.3 α subunit Antibodies,”), such as a monoclonal antibody or a single-chain antibody.

In another aspect, the invention features a method of analyzing the expression of mNaᵥ1.3 α subunit. The method includes providing an array as described above; contacting the array with a sample and detecting binding of mNaᵥ1.3 α subunit molecule (e.g., nucleic acid or polypeptide) to the array. Optionally the method further includes amplifying nucleic acid from the sample prior or during contact with the array.

In another embodiment, the array can be used to assay gene expression in a tissue to ascertain tissue specificity of genes in the array, particularly the expression of mNaᵥ1.3 α subunits. If a sufficient number of diverse samples is analyzed, clustering (e.g., hierarchical clustering, k-means clustering, Bayesian clustering and the like) can be used to identify other genes which are co-regulated with mNaᵥ1.3 α subunits. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on mNaᵥ1.3 α subunit expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

In another embodiment, cells are contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a disease or disorder associated with Na⁺ channel activity. The method can also evaluate the treatment and/or progression of a Na⁺ channel-associated disease or disorder

The array is also useful for ascertaining differential expression patterns of one or more genes in normal and abnormal cells. This provides a battery of genes (e.g., including genes encoding mNaᵥ1.3 α subunits) that could serve as a molecular target for diagnosis or therapeutic intervention.

In another aspect, the invention features an array having a plurality of addresses. Each address of the plurality includes a unique polypeptide. At least one address of the plurality has disposed thereon a mNaᵥ1.3 α subunit polypeptide or fragment thereof. Methods of producing polypeptide arrays are described in the art, e.g., in De Wildt et al., Nature Biotech. 18: 989-994, 2000; Lueking et al., Anal. Biochem. 270:103-111, 1999; Ge, H., Nucleic Acids Res. 28:e3, I-VII, 2000; MacBeath, G., and Schreiber, S. L., Science 289:1760-1763, 2000; and WO 99/51773A1. In a specific embodiment, each addresses of the plurality has disposed thereon a polypeptide at least 60%-99% identical to a mNaᵥ1.3 α subunit polypeptide or fragment thereof. For example, multiple variants of a mNaᵥ1.3 α subunit polypeptide (e.g., encoded by allelic variants, site-directed mutants, random mutants, or combinatorial mutants) can be disposed at individual addresses of the plurality.

The polypeptide array can be used to detect a Na⁺-binding compound, e.g., an antibody in a sample from a subject with specificity for a mNaᵥ1.3 α subunit polypeptide or the presence of a Na⁺ channel-binding protein or ligand.

In another aspect, the invention features a method of analyzing a plurality of probes. The method is useful, e.g., for analyzing gene expression. The method includes: providing a two-dimensional array having a plurality of addresses, each address of the plurality being positionally distinguishable from each other address of the plurality having a unique capture probe, e.g., wherein the capture probes are from a cell or subject which express Na⁺ channels comprising mNaᵥ1.3 α subunits or from a cell or subject in which a Na⁺ channel-mediated response has been elicited, e.g., by contact of the cell with Na⁺ channel mNaᵥ1.3 α subunit nucleic acids or proteins, or administration to the cell or subject Na⁺ channel mNaᵥ1.3 α subunit nucleic acids or proteins; providing a two dimensional array having a plurality of addresses, each address of the plurality being positionally distinguishable from each other address of the plurality, and each address of the plurality having a unique capture probe, e.g., wherein the capture probes are from a cell or subject which does not express Na⁺ channel mNaᵥ1.3 α subunits (or does not express as highly as in the case of the Na⁺ channel mNaᵥ1.3 α subunit-positive plurality of capture probes) or from a cell or subject which in which a Na⁺ channel-mediated response has not been elicited (or has been elicited to a lesser extent than in the first sample); contacting the array with one or more inquiry probes (which is preferably other than a Na⁺ channel mNaᵥ1.3 α subunit nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features a method of analyzing mNaᵥ1.3 α subunits, e.g., analyzing structure, function, or relatedness to other nucleic acid or amino acid sequences. The method includes: providing a mNaᵥ1.3 α subunit nucleic acid or amino acid sequence; comparing the sequence(s) with one or more preferably a plurality of sequences from a collection of sequences, e.g., a nucleic acid or protein sequence database; to thereby analyze mNaᵥ1.3 α subunit subunits.

### Detection Assays

Portions or fragments of the cDNA sequences identified herein (and the corresponding complete gene sequences) can be used in numerous ways as polynucleotide reagents.

For example, polynucleotide reagents can be used for diagnostic assays, prognostic assays, and monitoring clinical trials are used for prognostic (predictive) purposes to thereby treat an individual prophylactically. Accordingly, one aspect of the present invention relates to diagnostic assays for determining mNaᵥ1.3 α subunit protein and/or nucleic acid expression as well as mNaᵥ1.3 α subunit activity, in the context of a biological sample (e.g., blood, serum, cells, tissue) to thereby determine whether an individual is afflicted with a disease or disorder, or is at risk of developing a disorder, associated with aberrant or unwanted mNaᵥ1.3 α subunit expression or activity. The invention also provides for prognostic (or predictive) assays for determining whether an individual is at risk of developing a disorder associated with mNaᵥ1.3 α subunit protein, nucleic acid expression or activity. For example, mutations in a gene encoding a mNaᵥ1.3 α subunit can be assayed in a biological sample. and used for prognostic or predictive purposes.

Another aspect of the invention pertains to monitoring the influence of agents (e.g., drugs, compounds) on the expression or activity of mNaᵥ1.3 α subunit in vivo.

Monitoring the influence of agents (e.g., drugs) on the expression or activity of a mNaᵥ1.3 α subunit protein (e.g., the modulation of membrane excitability) can be applied not only in basic drug screening, but also in clinical trials. For example, the effectiveness of an agent determined by a screening assay as described herein to increase mNaᵥ1.3 α subunit gene expression, protein levels, or upregulate mNaᵥ1.3 α subunit activity, can be monitored in clinical trials of subjects exhibiting decreased or increased mNaᵥ1.3 α subunit gene expression, protein levels, or down-regulated mNaᵥ1.3 α subunit. Other genes that have been implicated in, for example, a Na⁺ channel associated disorder can be used markers of the phenotype of a particular cell.

### Therapeutic Methods

Another aspect of the invention pertains to methods of modulating mNaᵥ1.3 α subunit expression or activity for therapeutic purposes. Accordingly, in an exemplary embodiment, the modulatory method of the invention involves contacting a cell with a mNaᵥ1.3 α subunit or agent that modulates one or more of the activities of mNaᵥ1.3 α subunit protein activity associated with the cell. An agent that modulates mNaᵥ1.3 α subunit protein activity can be an agent as described herein, such as a nucleic acid or a protein, a naturally-occurring target molecule of a mNaᵥ1.3 α subunit protein (e.g., a mNaᵥ1.3 α subunit substrate), a mNaᵥ1.3 α subunit antibody, a mNaᵥ1.3 α subunit agonist or antagonist, a peptidomimetic of a mNaᵥ1.3 α subunit agonist or antagonist, or other small molecule. In one embodiment, the agent stimulates one or more mNaᵥ1.3 α subunit activities. Examples of such stimulatory agents include active mNaᵥ1.3 α subunit protein and a nucleic acid molecule encoding mNaᵥ1.3 α subunit that has been introduced into the cell. In another embodiment, the agent inhibits one or more mNaᵥ1.3 α subunit activities. Examples of such inhibitory agents include antisense mNaᵥ1.3 α subunit nucleic acid molecules, anti-mNaᵥ1.3 α subunit antibodies, and mNaᵥ1.3 α subunit inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a mNaᵥ1.3 α subunit protein or nucleic acid molecule. In one embodiment, the method involves administering an agent (e.g., an agent identified by a screening assay described herein), or combination of agents that modulates (e.g., upregulates or downregulates) mNaᵥ1.3 α subunit expression or activity. In another embodiment, the method involves administering a mNaᵥ1.3 α subunit protein or nucleic acid molecule as therapy to compensate for reduced or aberrant mNaᵥ1.3 α subunit expression or activity.

Stimulation of mNaᵥ1.3 α subunit activity is desirable in situations in which mNaᵥ1.3 α subunit is abnormally downregulated and/or in which increased mNaᵥ1.3 α subunit activity is likely to have a beneficial effect. Antagonism of activity may also be desirable. For example, modulators may be desirable for treatment of pain, e.g., neuropathic pain, head trauma, and neurodegenerative diseases. Neurodegenerative diseases include multiple sclerosis, Alzheimer's Disease, Parkinson's Disease or other forms of dementia, amyotrophic lateral sclerosis, Down's Syndrome, Huntington chorea and spinal cerebellar degeneration. Modulation of Naᵥ1.3 channels can be useful, e.g., in the treatment of various disorders accompanying cerebrovascular injury or trauma including cerebral hemorrhages such as hypertensive intracerebral hemorrhage and subarachnoid hemorrhage, transient cerebral ischemic attacks, cerebroarteriosclerosis and their sequela, as well as brain damages at the time of revivification after cardiac arrest, brain dysfunction prior to or after brain surgery, disorders of the nervous system due to hypoxia, hypoglycemia, brain or spinal damage, intoxication with drugs or gases, diabetes mellitus, administration of anti-cancer agents, alcohol and the like.

### Pharmaceutical Compositions

As used herein, the compounds of this invention, e.g., Na⁺ channel modulators identified by the methods described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including ion channel-mediated disorders or symptoms thereof.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried cornstarch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds and methods described above can be used for the therapeutic modulation of Na⁺ channel function.

Specific embodiments of the invention are further describe by way of the following non-limiting Examples:

### EXAMPLE 1

### Identification of a Novel Na⁺ Channel α Subunit

1. First, cDNA was generated from mouse brain polyA RNA (Stratagene and Clontech) using SuperScript First-Strand Synthesis System for RT-PCR (Invitrogen). Primers were designed for amplification of a mouse Naᵥ1.3 α subunit. A cDNA corresponding to a partial sequence of a mouse Naᵥ1.3 α subunit (GenBank® Acc. No. NM_{—}018732) aligns with the middle of the published rat cDNA sequence (NM_{—}013119; at nucleotide 2461 from the initiation codon). To identify the full length sequence for mouse Naᵥ1.3 α subunit, the rat sequence (NM_{—}013199) was compared to the mouse genome using BLAST® (Blake J A, et al. MGD: The Mouse Genome Database. Nucleic Acids Res 31: 193-195, 2003).
2. A 5′ primer was designed to anneal 7 nucleotides upstream of the putative initiation codon and a 3′ primer was designed to anneal 124 nucleotides downstream of the putative stop codon. Each primer contained a NotI restriction site at the end. Primers were designed to anneal to the untranslated regions. Primers that anneal to untranslated regions are less likely to amplify non-Naᵥ1.3 α subunit genes, i.e., mouse Naᵥ1.1 and Naᵥ1.2 α subunit genes. The 3′ primer that was used to amplify mNaᵥ1.3 was designed to anneal in an untranslated region corresponding to a region of high variability between rat Naᵥ1.1, Naᵥ1.2, and Naᵥ1.3 α subunit 3′ untranslated regions to increase specificity for mNaᵥ1.3. In this region in rat Naᵥ1 α subunit genes, rat Naᵥ1.1 differs from rat Naᵥ1.2 by 17 out of 32 nucleotides, and rat Naᵥ1.2 differs from rat Naᵥ1.3 by 13 out of 32 nucleotides. This untranslated region also has a high degree of homology between rat and murine sequences; only 29 of 32 nucleotides are conserved.
3. Cloning of a mouse Naᵥ1.3 α subunit cDNA encoding a mouse Naᵥ1.3 α subunit was amplified from mouse brain cDNA using Herculase Hotstart DNA Polymerase (Stratagene; 30 cycles). The PCR product was resolved by agarose gel electrophoresis and extracted from the gel using Qiaquick Gel Extraction kit (Qiagen). The extracted product was subdloned into pCR-XL-TOPO vector using the TOPO XL PCR Cloning Kit (Invitrogen). The resulting vectors were transformed into XL 10 Gold Ultracompetent Cells (Stratagene). Transformed bacteria were cultured. DNA was isolated from the transformed bacteria using an Aurum Miniprep (Biorad).

Clones containing a mouse Naᵥ1.3 α subunit insert were identified by agarose gel analysis of uncut DNA and NotI (New England Biolabs) digested DNA. Sequence analysis of the 5′ and 3′ ends of two clones (A and B; SeqWright) was performed to verify the presence of the mouse Naᵥ1.3 α subunit insert

Bacteria containing the clones A and B were cultured for preparation of a larger quantity of DNA by maxi prep (Qiagen).

4. Full length sequencing of clones A and B (SeqWright) was performed. To determine if PCR errors were present in clones A and B, the nucleotide sequence of the two clones were compared to each other, to the mouse Naᵥ1.3 α subunit genomic sequence, to the sequence of the rat Naᵥ1.3 α subunit cDNA (NM_{—}013199) and to two human Naᵥ1.3 α subunit cDNAs (NM_{—}006922 and AF225986). In addition, the predicted amino acid sequences encoded by nucleotide sequence of clones A and B were generated and compared with the rat and human Naᵥ1.3 α subunit protein sequences.

Clone A contained one nucleotide change relative to the murine genomic sequence and a human Naᵥ1.3 α subunit sequence. Clone A also contained an 83 nucleotide deletion relative to clone B.

Clone A and clone B both contained an extra A in a string of six As which was most likely introduced by PCR error. The presence of this nucleotide in a coding sequence would shift the reading frame and encode a protein that is 1504 amino acids (as opposed to approximately 1940 amino acids in known Naᵥ α subunit polypeptides). Also, the extra A is not present in the genomic murine sequence. The rat and human sequences also lack an extra nucleotide at that position.

Clone B contained two nucleotide changes that were not present in clone A or corresponding genomic regions in murine, rat, and human genomic sequences, and thus were determined to be errors. Clone B also had an extra nucleotide that was not present in clone A or in the corresponding position in murine, rat, and human genomic sequences, which was also determined to be an error.

5. Repair of the mutations found in the mouse Naᵥ1.3 α subunit clones has been performed with the following steps. For step 1 of the repair, the mutation and the 83 nucleotide deletion in clone A were corrected by replacing the mutated fragment with the corresponding fragment from clone B as follows. Clones A and B were digested with XcmI (New England Biolabs). The large fragment of clone A (which lacks the mutation and deletion) and the small fragment of clone B (to replace the fragment from clone A) were resolved and extracted from an agarose gel using a Qiaquick Gel Extraction kit. The digested fragments were ligated using T4 DNA ligase (New England Biolabs). The ligated product was transformed into MAX Efficiency Stbl2 Competent Cells (Invitrogen). Bacteria were cultured and DNA was isolated with an Aurum miniprep. Clones containing the appropriate XcmI fragment were identified by agarose gel analysis of uncut DNA and DNA digested with SmaI and NarI (New England Biolabs).

Sequence analysis of several clones (SeqWright) was performed to identify DNA in which the mutation and 83 nucleotide deletion was repaired and to verify the presence of the two extra nucleotides (one extra nucleotide was present in clone A and one was carried over with the XcmI fragment from clone B).

6. For step 2 of the repair of the isolated mouse Naᵥ1.3 α subunit cDNA, a clone in which the mutation and 83 nucleotide deletion had been repaired was used. One extra nucleotide in this clone was corrected with a QuickChange XL Site-Directed Mutagenesis Kit (Stratagene). Site-directed mutagenesis primers were designed. The mutagenesis reaction was performed, and the product of the reaction was transformed into XL10 Gold Ultracompetent Cells (Stratagene). Transformed bacteria were cultured and DNA was isolated by Aurum miniprep. Clones were analyzed with SmaI digestion of DNA. Sequence analysis of several clones was performed to identify DNA in which the extra nucleotide was repaired. Bacteria from one corrected clone were regrown so the insert containing the extra nucleotide could be transferred to pcDNA6B and repaired.
7. To transfer the mouse Naᵥ1.3 α subunit cDNA that contained one extra nucleotide from pCR-XL-TOPO into pcDNA6B, the clone was digested with EcoRV and SpeI (New England Biolabs). pcDNA6B was digested with EcoRV and XbaI (New England Biolabs). Digested fragments were resolved and extracted from an agarose gel using a Quiaquick Gel Extraction kit and ligated using T4 DNA ligase. The ligated product was transformed into XL10 Gold Ultracompetent Cells. Bacteria were cultured and DNA was isolated with an Aurum miniprep. Clones containing mouse Naᵥ1.3 α subunit were identified by agarose gel analysis of uncut DNA and SmaI digested DNA. One clone was regrown for the final step of repair.
8. For step 3 of the repair process, a fragment containing the extra nucleotide in mouse Naᵥ1.3 α subunit in pcDNA6B was replaced with a newly amplified fragment as follows. Primers were designed to amplify an internal fragment of mouse Naᵥ1.3 α subunit (nucleotide 3950-4675 from the initiation codon). The fragment was amplified from mouse brain cDNA (generated in step 1) with PfuTurbo Hotstart DNA Polymerase (Stratagene), resolved by agarose gel electrophoresis and extracted from the agarose gel using a Quiaquick Gel Extraction kit. The fragment was subdloned into a pCR-XL-TOPO vector and transformed into One Shot TOP10 Competent Cells (Invitrogen). Transformed bacteria were cultured and DNA was isolated by an Aurum miniprep. Clones containing the mouse Naᵥ1.3 α subunit fragment were identified by agarose gel analysis of DNA digested with EcoRI and double digested with ScaI and BglI (New England Biolabs). Sequence analysis of several clones was performed to identify DNA clones lacking mutations. One clone was regrown.

Replacement of the fragment containing an extra nucleotide in mouse Naᵥ1.3 α subunit in pcDNA6B with the corrected fragment from pCR-XL-TOPO was performed as follows. The two clones were digested with HpaI and BstEII (New England Biolabs). Digested fragments were resolved and extracted from an agarose gel using a Quiaquick Gel Extraction kit. The fragments were ligated using T4 DNA ligase and the ligated product was transformed into MAX Efficiency Stbl2 Competent Cells. Bacteria were cultured and DNA was isolated with an Aurum miniprep. Clones were analyzed by restriction digestion with HpaI, BstEII and SmaI. Sequencing of the “swapped” fragment (SeqWright) of several clones was performed to identify clones in which the extra nucleotide was repaired. Full length sequencing of a single clone was performed to verify that no new mutations were produced during the site-directed mutagenesis procedure (step 2 of repair).

### EXAMPLE 2

### Determination of Coding Sequences

Table 1. depicts the coding sequence of the mouse Naᵥ1.3 α subunit. The coding sequence of the cDNA corresponds to nucleotide 8 through nucleotide 5947 of the sequence shown in Table 1. SEQ ID NO:1 corresponds to the coding sequence portion of the sequence shown in Table 1. The predicted amino acid sequence of SEQ ID NO:1 is shown in SEQ ID NO:2 and Table 2. SEQ ID NO:3 corresponds to the entire sequence shown in Table 1. The bold, underlined ATG sequence at nucleotides 8-10 and the bold, underlined TGA sequence at nucleotides 5945-5947 represent the initial and final codons of the coding sequence, respectively.

TABLE 1
Nucleotide sequence of mouse Naᵥ1.3 α subunit (SEQ ID NO:3)

| | |
|---|---|
| 1 | TGAAAAGATG GCCCAGGCAC TGCTGGTACC CCCGGGACCT GAGAGCTTCC |
| 51 | GCCTTTTCAC TCGAGAATCT CTTGCTGCTA TCGAAAAGCG TGCTGCAGAA |
| 101 | GAGAAAGCCA AGAAACCCAA GAAAGAACAA GACATTGACG ATGAGAACAA |
| 151 | GCCAAAGCCA AACAGTGACT TGGAAGCTGG GAAGAACCTT CCATTTATCT |
| 201 | ATGGAGACAT TCCTCCAGAG ATGGTGTCGG AGCCTCTGGA GGACCTGGAC |
| 251 | CCCTACTACG TCAGTAAGAA AACTTTTGTA GTGTTGAATA AAGGGAAGGC |
| 301 | AATTTTTCGA TTCAGTGCCA CCTCCGCCTT GTATATTTTA ACTCCACTAA |
| 351 | ACCCCGTTAG GAAAATTGCT ATTAAGATTT TGGTACACTC TTTATTCAGC |
| 401 | ATGCTTATCA TGTGCACTAT TTTGACCAAC TGTGTATTTA TGACATTGAG |
| 451 | CAATCCTCCT GACTGGACGA AGAATGTAGA GTACACATTC ACTGGGATCT |
| 501 | ATACCTTTGA GTCACTTATA AAGATCTTGG CCAGAGGATT CTGCTTAGAA |
| 551 | GATTTCACAT TTCTTCGTGA CCCATGGAAC TGGCTGGATT TCAGTGTCAT |
| 601 | CGTGATGGCA TATGTGACAG AGTTTGTGGA CCTGGGCAAT GTCTCAGCGC |
| 651 | TGAGAACGTT CAGAGTTCTC CGAGCATTGA AAACAATATC AGTCATTCCA |
| 701 | GGTTTAAAGA CCATCGTGGG GGCCCTGATC CAGTCGGTGA AGAAGCTGTC |
| 751 | TGACGTCATG ATACTCACTG TGTTCTGTCT GAGCGTCTTT GCTCTCATCG |
| 801 | GGCTGCAGCT CTTCATGGGC AACCTGAGGA ATAAATGCTT GCAGTGGCCT |
| 851 | CCAAGCGATT CTGCTTTTGA GATCAACACT ACTTCCTACT TCAATGGCAC |
| 901 | AATGGACTCA AATGGGACAT TTGTTAATGT AACAATGAGC ACATTCAACT |
| 951 | GGAAGGACTA TATCGCAGAT GACAGCCACT TTTATGTTTT GGATGGACAA |
| 1001 | AAAGATCCTT TACTTTGTGG AAATGGGTCC GATGCAGGAC AATGTCCAGA |
| 1051 | AGGGTACATC TGTGTGAAGG CTGGACGAAA CCCCAACTAC GGTTACACGA |
| 1101 | GCTTTGACAC ATTTAGCTGG GCCTTCTTAT CGCTGTTTCG ACTCATGACT |
| 1151 | CAAGACTACT GGGAGAACCT TTACCAGTTG ACATTACGTG CAGCTGGGAA |
| 1201 | AACCTACATG ATCTTTTTCG TCCTGGTAAT TTTCTTGGGC TCATTTTATT |
| 1251 | TGGTGAACTT GATCCTGGCT GTGGTGGCCA TGGCCTATGA GGAACAAAAT |
| 1301 | CAGGCCACAC TGGAGGAGGC TGAGCAGAAA GAGGCGGAGT TTCAGCAGAT |
| 1351 | GTTGGAGCAG TTGAAAAAGC AGCAAGAGGA GGCTCAGGCG GTGGCAGCTG |
| 1401 | CCTCAGCAGC GTCCAGAGAC TTCAGTGGGA TAGGAGGGTT AGGAGAACTT |
| 1451 | CTGGAGAGTT CTTCAGAAGC TTCCAAGTTG AGCTCCAAGA GTGCCAAGGA |
| 1501 | GTGGAGGAAT CGGAGGAAGA AGAGGAGACA GAGGGAGCAC TTGGAGGGAA |
| 1551 | ACCACAGACC CGAAGGAGAC AGGTTTCCCA AGTCGGAATC AGAAGACAGC |
| 1601 | GTCAAGCGAA GGAGTTTCCT GTTCTCCCTG GATGGGAACC CGCTGAGCGG |
| 1651 | CGACAAGAAG CTGTGCTCTC CCCATCAGTC TCTCTTGAGT ATCCGTGGCT |
| 1701 | CCCTGTTTTC CCCAAGACGC AATAGCAAAA CGAGCATTTT CAGCTTCAGA |
| 1751 | GGTCGGGCGA AGGACGTGGG GTCTGAGAAT GACTTTGCGG ATGATGAACA |
| 1801 | CAGCACCTTT GAAGATAGCG AGAGCAGGAG AGACTCACTG TTTGTGCCGC |
| 1851 | ACAGACCTGG AGAGCGACGC AACAGTAACG TTAGTCAGGC CAGTATGTCA |
| 1901 | TCCAGGATGG TGCCAGGGCT TCCAGCCAAT GGGAAGATGC ACAGCACTGT |
| 1951 | GGATTGCAAT GGTGTGGTTT CCTTGGGTAC CACCACTGAA ACAGAAGTCA |
| 2001 | GGAAGAGAAG GCTAAGTTCT TACCAGATCT CGATGGAAAT GCTGGAGGAT |
| 2051 | TCCTCTGGGA GACAAAGAGC CATGAGCATA GCCAGTATCC TGACCAACAC |
| 2101 | GATGGAGGAA CTTGAAGAAT CTAGACAGAA GTGTCCACCA TGCTGGTATA |
| 2151 | GATTTGCCAA TGTGTTTTTG ATCTGGGACT GTTGTGATTC ATGGTTGAAA |
| 2201 | GTAAAGCATC TTGTGAATTT GATTGTGATG GATCCATTTG TTGACCTGGC |
| 2251 | CATCACCATC TGCATCGTGT TAAACACACT GTTCATGGCC ATGGAGCACT |
| 2301 | ACCCGATGAC GGAGCAGTTC AGCAGTGTGC TGACGGTGGG AAACCTGGTC |
| 2351 | TTCACCGGGA TCTTCACAGC CGAGATGGTC CTGAAAATCA TCGCAATGGA |
| 2401 | TCCCTATTAC TATTTCCAAG AGGGCTGGAA TATCTTTGAT GGAATTATTG |
| 2451 | TTAGCCTGAG TTTAATGGAG CTTGGCCTGG CAAACGTGGA GGGGCTGTCC |
| 2501 | GTGCTTCGGT CCTTCAGACT GCTGCGAGTC TTCAAGTTGG CAAAATCCTG |
| 2551 | GCCCACACTG AATATGCTCA TTAAGATCAT CGGCAACTCG GTGGGCGCAC |
| 2601 | TGGGCAACCT GACCCTGGTG CTGGCCATCA TCGTCTTCAT TTTTGCCGTG |
| 2651 | GTCGGCATGC AGCTGTTTGG AAAGAGCTAC AAGGAGTGTG TTTGCAAGAT |
| 2701 | CAATGAGGAC TGCAAGCTCC CGCGCTGGCA CATGAACGAC TTCTTCCACT |
| 2751 | CCTTCCTGAT AGTGTTCCGC GTGCTGTGTG GGGAGTGGAT AGAGACCATG |
| 2801 | TGGGACTGCA TGGAGGTCGC GGGCCAGACC ATGTGCCTTA TTGTGTTTAT |
| 2851 | GTTGGTCATG GTGATTGGGA ACCTTGTGGT TCTGAACCTC TTCCTGGCCT |
| 2901 | TATTGTTGAG TTCCTTTAGT TCAGACAACC TTGCTGCTAC GGACGATGAT |
| 2951 | AACGAAATGA ACAACCTCCA GATCGCGGTG CGAAGGATGC AAAAGGGGAT |
| 3001 | TGATTATGTG AAAAATAAGA TACGGGAGTG CTTCCGAAAA GCGTTTTTTA |
| 3051 | GAAAGCCGAA AGTGATAGAA ATCCACGAAG GGAACAAAAT AGACAGCTGC |
| 3101 | ATGTCCAATA ACACGGGCGT AGTTGAAATA AGCAAAGAGC TTAACTACCT |
| 3151 | TAAAGACGGT AACGGAACCA CCAGTGGCGT GGGTACTGGA AGCAGTGTGG |
| 3201 | AGAAATACGT AATTGATGAA AATGACTACA TGTCATTCAT CAACAACCCC |
| 3251 | AGCCTCACCG TGACGGTGCC AATTGCCGTG GGAGAGTCTG ACTTTGAAAA |
| 3301 | TTTAAACACG GAAGAGTTTA GCAGTGAGTC AGAACTGGAA GAAAGCAAGG |
| 3351 | AGAAATTAAA TGCAACCAGC TCTTCTGAAG GAAGCACAGT TGACGTTGCT |
| 3401 | CCGCCCCGAG AAGGTGAACA AGCTGAAATT GAACCTGAGG AGGACCTTAA |
| 3451 | GCCAGAAGCT TGCTTTACTG AAGGATGCAT TAAAAAATTT CCCTTCTGCC |
| 3501 | AAGTAAGTAC GGAAGAAGGT AAAGGAAAAA TCTGGTGGAA TCTTAGGAAG |
| 3551 | ACCTGCTATA GCATTGTGGA ACACAACTGG TTTGAGACGT TCATTGTGTT |
| 3601 | CATGATTCTC CTCAGTAGTG GTGCTTTGGC CTTTGAAGAT ATATACATTG |
| 3651 | AGCAACGGAA GACCATCAAA ACCATGCTGG AGTATGCTGA CAAAGTCTTC |
| 3701 | ACTTACATCT TCATCCTGGA AATGCTCCTC AAATGGGTGG CCTATGGATT |
| 3751 | TCAAACCTAT TTCACCAATG CCTGGTGCTG GTTGGACTTC TTGATTGTTG |
| 3801 | ATGTTTCTTT GGTTAGCCTG GTGGCCAACG CTCTTGGCTA TTCAGAACTT |
| 3851 | GGTGCCATCA AATCCCTACG GACCCTGAGA GCTCTGAGGC CGCTCCGAGC |
| 3901 | CTTATCCCGC TTTGAAGGCA TGAGGGTGGT TGTGAACGCT CTTGTTGGTG |
| 3951 | CAATCCCCTC CATCATGAAT GTGCTACTGG TGTGCCTCAT CTTCTGGTTA |
| 4001 | ATCTTTAGTA TCATGGGTGT GAATCTGTTT GCTGGAAAGT TCTATCACTG |
| 4051 | TGTTAACATG ACAACGGGCA GCATGTTCGA CATGAGTGAA GTCAACAATT |
| 4101 | TCAGCGACTG TCAGGCTCTT GGCAAGCAAG CCCGATGGAA GAATGTGAAA |
| 4151 | GTCAACTTTG ACAATGTTGG GGCTGGCTAC CTGGCATTGC TGCAAGTGGC |
| 4201 | CACATTCAAA GGCTGGATGG ATATTATGTA TGCAGCTGTG GATTCACGGG |
| 4251 | ACGTCAAACT GCAGCCTGTA TATGAAGAAA ATCTGTACAT GTATCTGTAC |
| 4301 | TTTGTCATCT TCATCATCTT TGGGTCGTTC TTCACTCTAA ATCTATTCAT |
| 4351 | CGGCGTCATC ATAGACAACT TCAACCAGCA GAAGAAGAAG TTTGGAGGTC |
| 4401 | AAGACATCTT TATGACAGAA GAGCAGAAAA AGTACTACAA TGCAATGAAG |
| 4451 | AAACTTGGCT CCAAAAAACC TCAGAAGCCC ATCCCTCGAC CTGCAAACAA |
| 4501 | ATTTCAAGGA ATGGTCTTTG ACTTTGTAAC CAGACAAGTG TTTGACATCA |
| 4551 | GCATCATGAT CCTCATCTGC CTCAACATGG TGACCATGAT GGTGGAAACG |
| 4601 | GACGACCAGA GCAAATACAT GACCCTGGTT TTGTCCCGAA TCAACCTGGT |
| 4651 | ATTCATCGTC CTCTTCACTG GGGAGTTTCT GCTGAAGCTC ATCTCTCTCA |
| 4701 | GATACTACTA CTTCACGATT GGCTGGAACA TCTTTGACTT TGTGGTGGTG |
| 4751 | ATTCTCTCAA TTGTAGGAAT GTTCCTTGCT GAGCTGATAG AGAAGTATTT |
| 4801 | TGTGTCTCCT ACCCTGTTCC GAGTCATCCG CCTGGCCAGG ATTCGACGAA |
| 4851 | TCCTACGCCT GATCAAAGGC GCCAAGGGGA TCCGCACGCT GCTCTTTGCT |
| 4901 | CTGATGATGT CCCTTCCTGC GCTGTTCAAC ATCGGCCTCC TGCTTTTCCT |
| 4951 | CGTCATGTTC ATCTACGCCA TCTTTGGGAT GTCCAACTTT GCCTATGTTA |
| 5001 | AAAAAGAGGC TGGAATTGAT GACATGTTCA ACTTTGAGAC TTTTGGCAAC |
| 5051 | AGCATGATCT GCCTGTTCCA AATCACCACC TCTGCGGGCT GGGATGGACT |
| 5101 | GTTGGCCCCC ATCCTCAACA GTGCACCTCC TGACTGTGAC CCTGATGCAA |
| 5151 | TTCACCCTGG AAGCTCAGTG AAGGGAGACT GTGGGAACCC ATCTGTGGGG |
| 5201 | ATTTTCTTTT TTGTCAGCTA CATCATCATA TCCTTCCTGG TTGTGGTGAA |
| 5251 | CATGTACATT GCTGTCATCC TGGAGAACTT CAGCGTTGCC ACAGAAGAAA |
| 5301 | GTGCAGAGCC CCTGAGTGAG GACGACTTTG AGATGTTCTA CGAGGTCTGG |
| 5351 | GAGAAGTTCG ACCCTGACGC CACCCAGTTC ATAGAGTTCT GCAAGCTCTC |
| 5401 | TGACTTTGCA GCTGCCCTGG ATCCTCCCCT CCTCATCGCA AAGCCAAACA |
| 5451 | AAGTCCAGCT CATTGCCATG GACCTGCCCA TGGTGAGTGG AGACCGCATC |
| 5501 | CACTGCCTGG ACATCTTATT TGCTTTTACA AAGCGGGTCC TGGGTGAGAG |
| 5551 | TGGAGAGATG GATGCCCTTC GAATCCAGAT GGAAGATCGG TTCATGGCTT |
| 5601 | CCAATCCCTC CAAGGTCTCT TATGAGCCCA TTACCACCAC TCTGAAGCGC |
| 5651 | AAACAAGAGG AGGTGTCTGC TGCTATCATT CAGCGTAATT ACAGATGTTA |
| 5701 | TCTTTTAAAG CAAAGGTTAA AAAACATATC AAATACGTAT GACAAAGAGA |
| 5751 | CAATCAAGGG GAGGATTGTC TTGCCTATAA AAGGAGATAT GGTTATTGAC |
| 5801 | AAATTAAATG GGAATTCCAC CCCAGAAAAG ACAGATGGGA GTTCCTCTAC |
| 5851 | CACCTCCCCT CCTTCCTATG ACAGTGTAAC AAAACCAGAT AAGGAAAAGT |
| 5901 | TTGAGAAAGA CAAACCAGAA AAAGAAAGCA AAGGGAAAGA GGTCTGAGAG |
| 5951 | AATCAAAAGT AAAAAAACAA AACAAAAAAA ATTTCAAAAA TTTAAAAAGG |
| 6001 | AAACAAAGAA ATGTCTTTGT AATCAATTGT TTACAGCCTC TGAAGGTAAA |
| 6051 | GTGTCCGTGT CAACTGGACT C |

TABLE 2

Amino acid sequence of mNaᵥ1.3 α subunit
(SEQ ID NO:2)
MAQALLVPPGPESFRLFTRESLAAIEKRAAEEKAKKPKKEQDIDDENKPK
PNSDLEAGKNLPFIYGDIPPEMVSEPLEDLDPYYVSKKTFVVLNKGKAIF
RFSATSALYILTPLNPVRKIAIKILVHSLFSMLIMCTILTNCVFMTLSNP
PDWTKNVEYTFTGIYTFESLIKILARGFCLEDFTFLRDPWNWLDFSVIVM
AYVTEFVDLGNVSALRTFRVLRALKTISVIPGLKTIVGALIQSVKKLSDV
MILTVFCLSVFALIGLQLFMGNLRNKCLQWPPSDSAFEINTTSYFNGTMD
SNGTFVNVTMSTFNWKDYIADDSHFYVLDGQKDPLLCGNGSDAGQCPEGY
ICVKAGRNPNYGYTSFDTFSWAFLSLFRLMTQDYWENLYQLTLRAAGKTY
MIFFVLVIFLGSFYLVNLILAVVAMAYEEQNQATLEEAEQKEAEFQQMLE
QLKKQQEEAQAVAAASAASRDFSGIGGLGELLESSSEASKLSSKSAKEWR
NRRKKRRQREHLEGNHRPEGDRFPKSESEDSVKRRSFLFSLDGNPLSGDK
KLCSPHQSLLSIRGSLFSPRRNSKTSIFSFRGRAKDVGSENDFADDEHST
FEDSESRRDSLFVPHRPGERRNSNVSQASMSSRMVPGLPANGKMHSTVDC
NGVVSLGTTTETEVRKRRLSSYQISMEMLEDSSGRQRAMSIASILTNTME
ELEESRQKCPPCWYRFANVFLIWDCCDSWLKVKHLVNLIVMDPFVDLAIT
ICIVLNTLFMAMEHYPMTEQFSSVLTVGNLVFTGIFTAEMVLKIIAMDPY
YYFQEGWNIFDGIIVSLSLMELGLANVEGLSVLRSFRLLRVFKLAKSWPT
LNMLIKIIGNSVGALGNLTLVLAIIVFIFAVVGMQLFGKSYKECVCKINE
DCKLPRWHMNDFFHSFLIVFRVLCGEWIETMWDCMEVAGQTMCLIVFMLV
MVIGNLVVLNLFLALLLSSFSSDNLAATDDDNEMNNLQIAVGRMQKGIDY
VKNKIRECFRKAFFRKPKVIEIHEGNKIDSCMSNNTGVVEISKELNYLKD
GNGTTSGVGTGSSVEKYVIDENDYMSFINNPSLTVTVPIAVGESDFENLN
TEEFSSESELEESKEKLNATSSSEGSTVDVAPPREGEQAEIEPEEDLKPE
ACFTEGCIKKFPFCQVSTEEGKGKIWWNLRKTCYSIVEHNWFETFIVFMI
LLSSGALAFEDIYIEQRKTIKTMLEYADKVFTYIFILEMLLKWVAYGFQT
YFTNAWCWLDFLIVDVSLVSLVANALGYSELGAIKSLRTLRALRPLRALS
RFEGMRVVVNALVGAIPSIMNVLLVCLIFWLIFSIMGVNLFAGKFYHCVN
MTTGSMFDMSEVNNFSDCQALGKQARWKNVKVNFDNVGAGYLALLQVATF
KGWMDIMYAAVDSRDVKLQPVYEENLYMYLYFVIFIIFGSFFTLNLFIGV
IIDNFNQQKKKFGGQDIFMTEEQKKYYNAMKKLGSKKPQKPIPRPANKFQ
GMVFDFVTRQVFDISIMILICLNMVTMMVETDDQSKYMTLVLSRINLVFI
VLFTGEFLLKLISLRYYYFTIGWNIFDFVVVILSIVGMFLAELIEKYFVS
PTLFRVIRLARIGRILRLIKGAKGIRTLLFALMMSLPALFNIGLLLFLVM
FIYAIFGMSNFAYVKKEAGIDDMFNFETFGNSMICLFQITTSAGWDGLLA
PILNSAPPDCDPDAIHPGSSVKGDCGNPSVGIFFFVSYIIISFLVVVNMY
IAVILENFSVATEESAEPLSEDDFEMFYEVWEKFDPDATQFIEFCKLSDF
AAALDPPLLIAKPNKVQLIAMDLPMVSGDRIHCLDILFAFTKRVLGESGE
MDALRIQMEDRFMASNPSKVSYEPITTTLKRKQEEVSAAIIQRNYRCYLL
KQRLKNISNTYDKETIKGRIVLPIKGDMVIDKLNGNSTPEKTDGSSSTTS
PPSYDSVTKPDKEKFEKDKPEKESKGKEV

### EXAMPLE 3

### Expression of a Novel Na⁺ Channel α Subunit

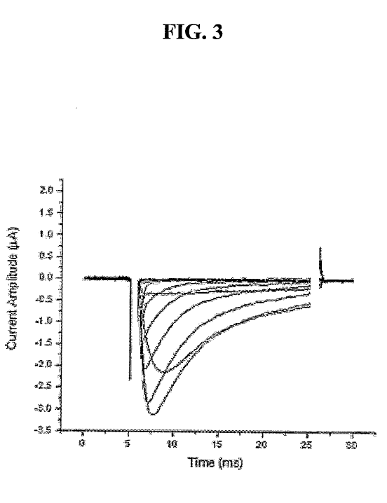

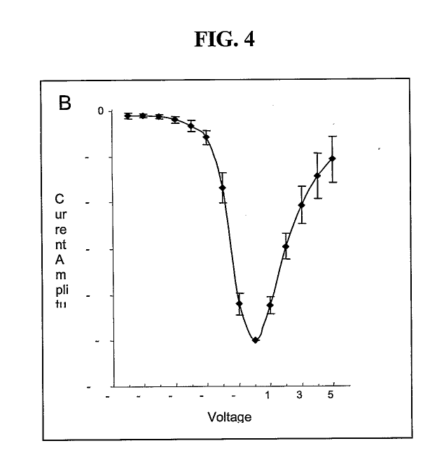

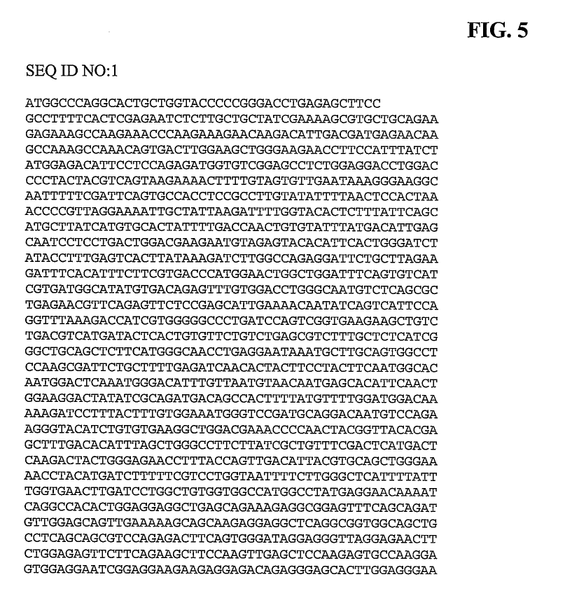

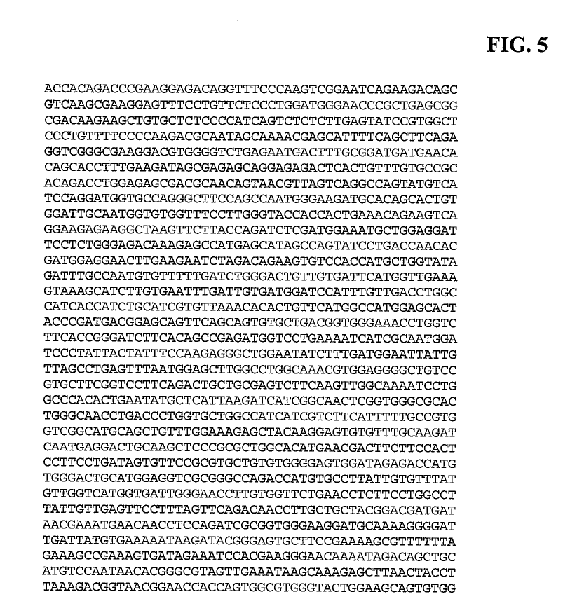

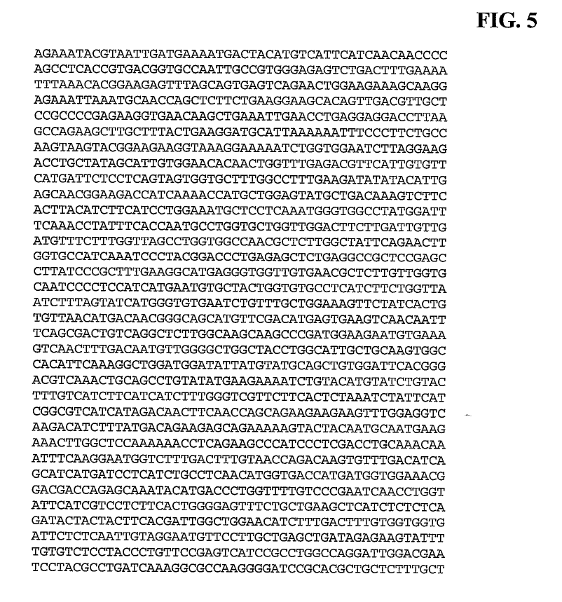

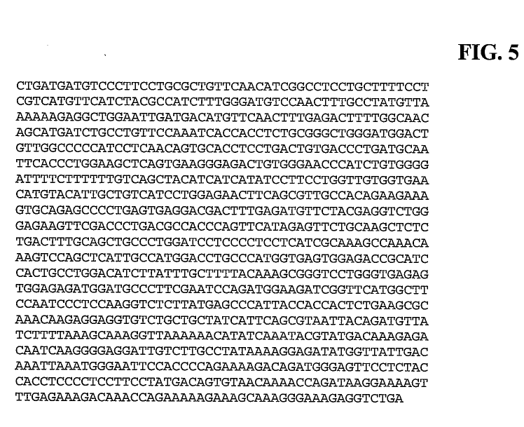

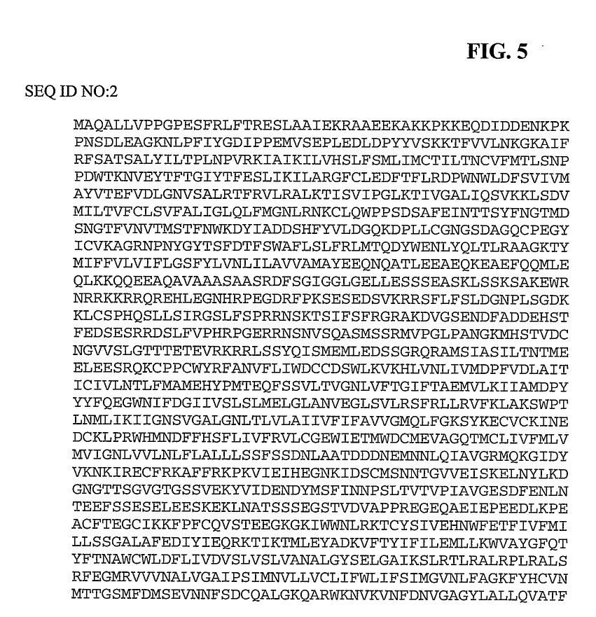

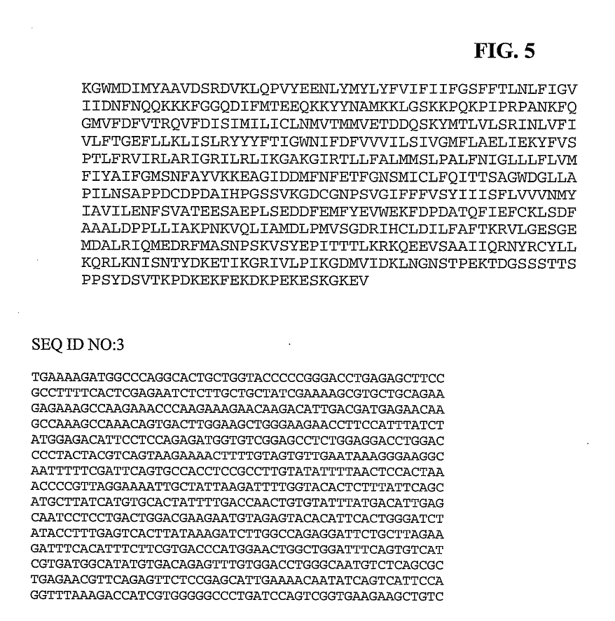

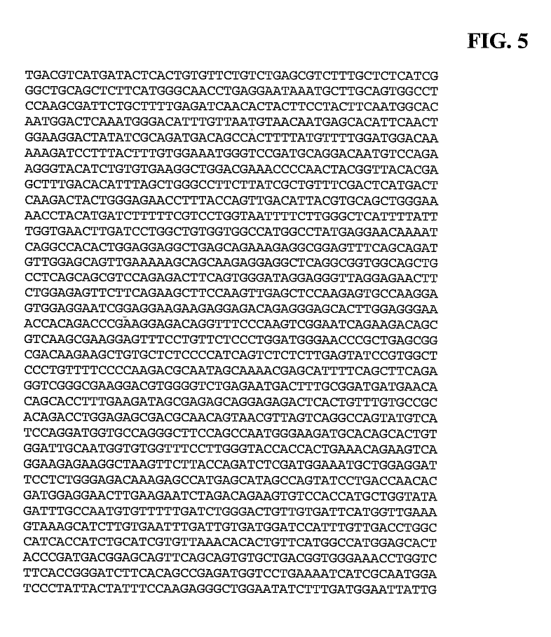

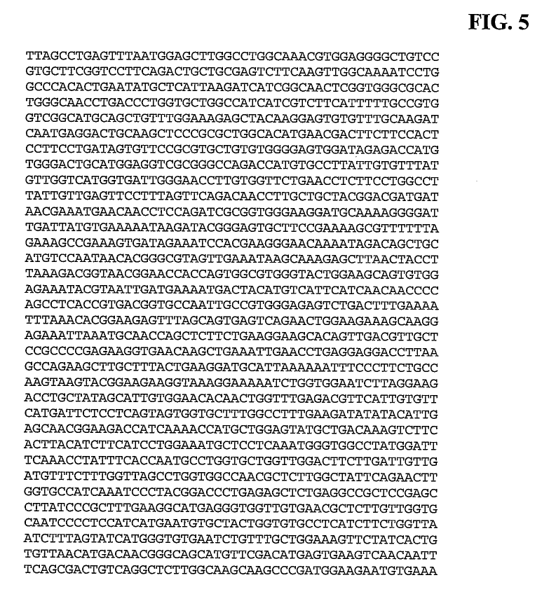

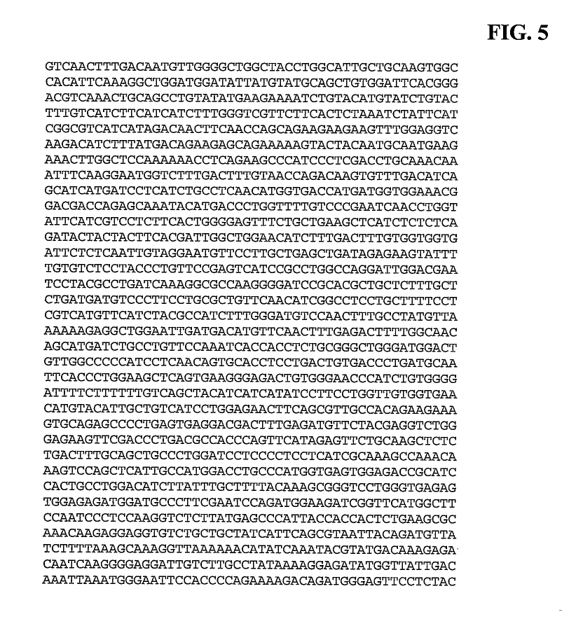

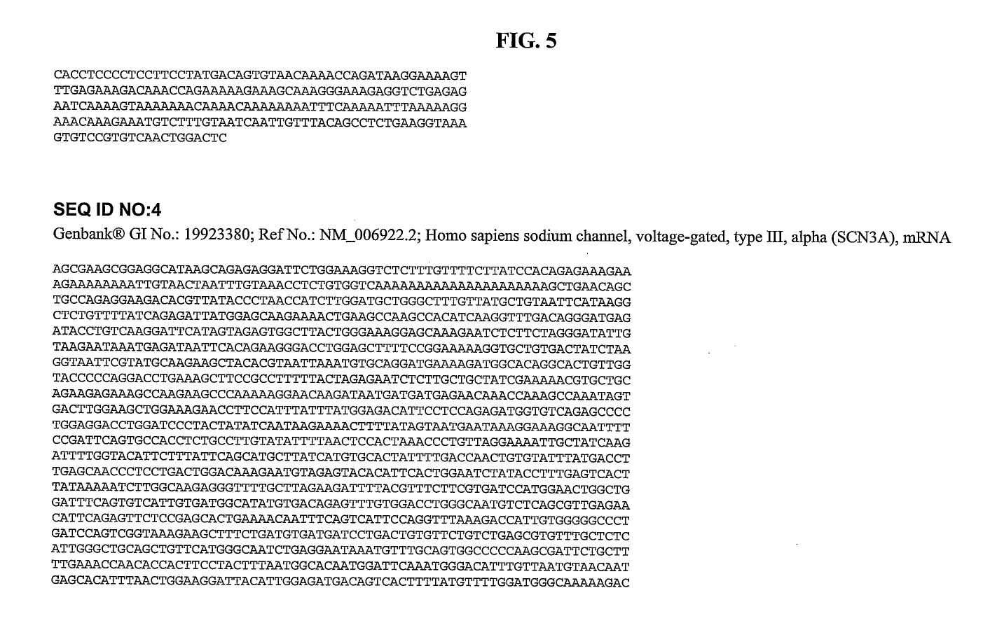

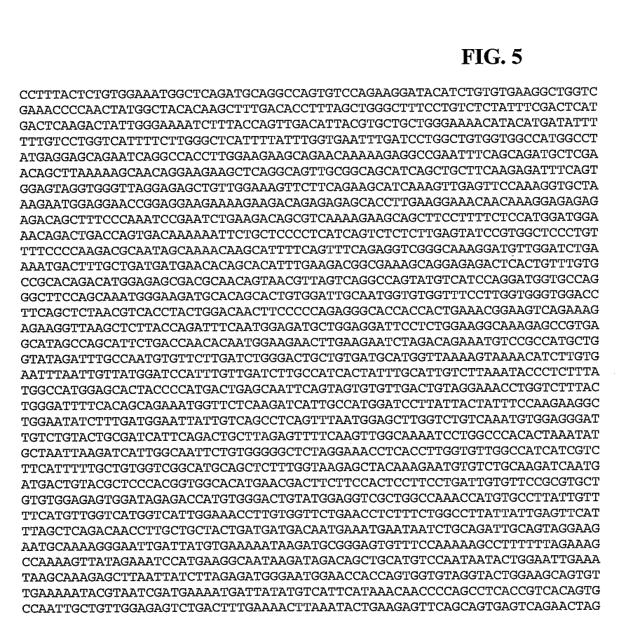

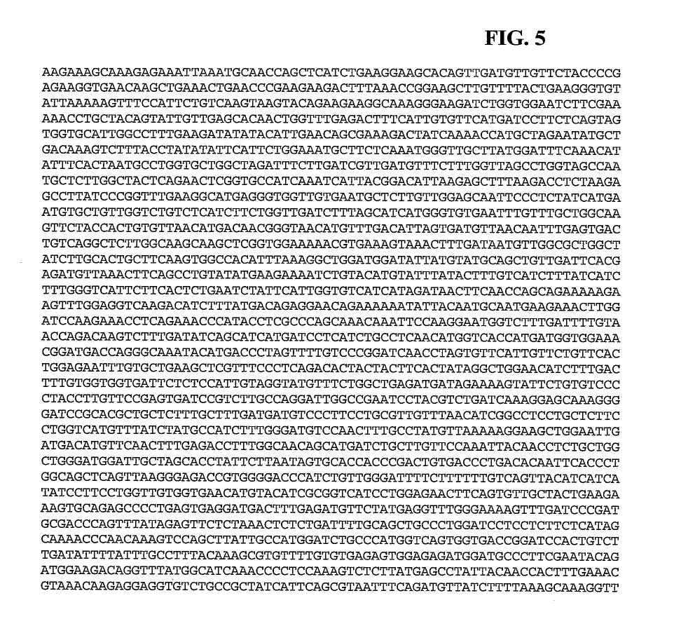

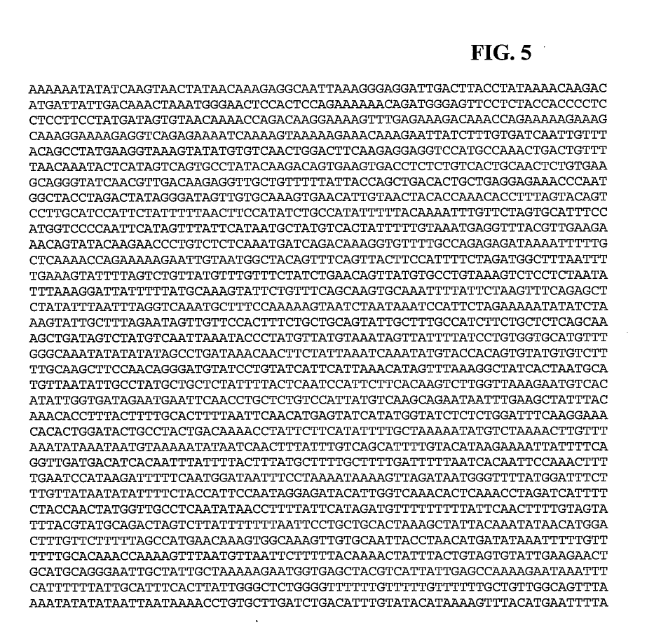

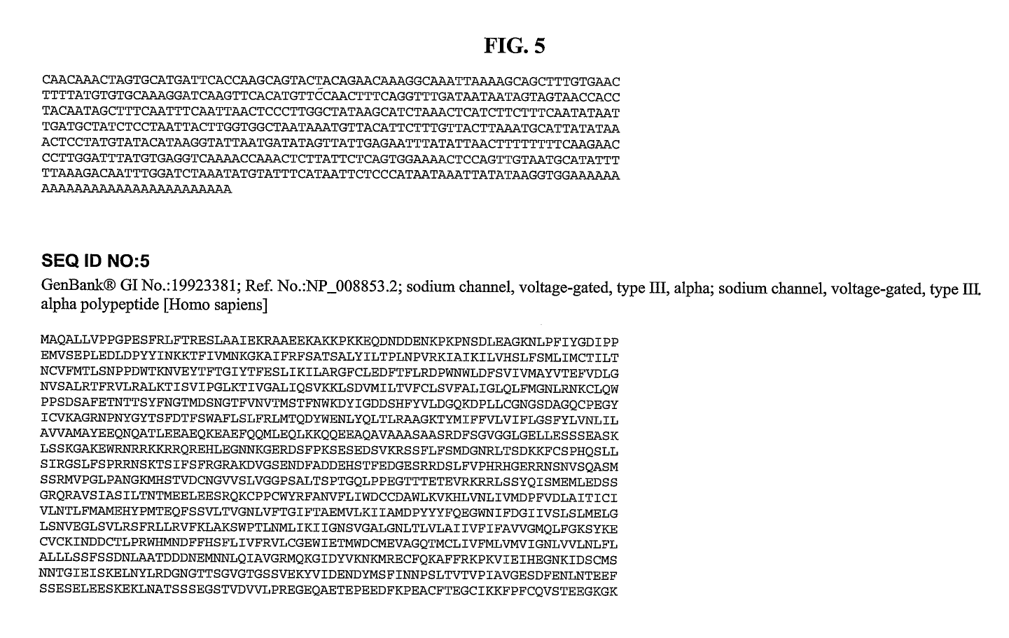

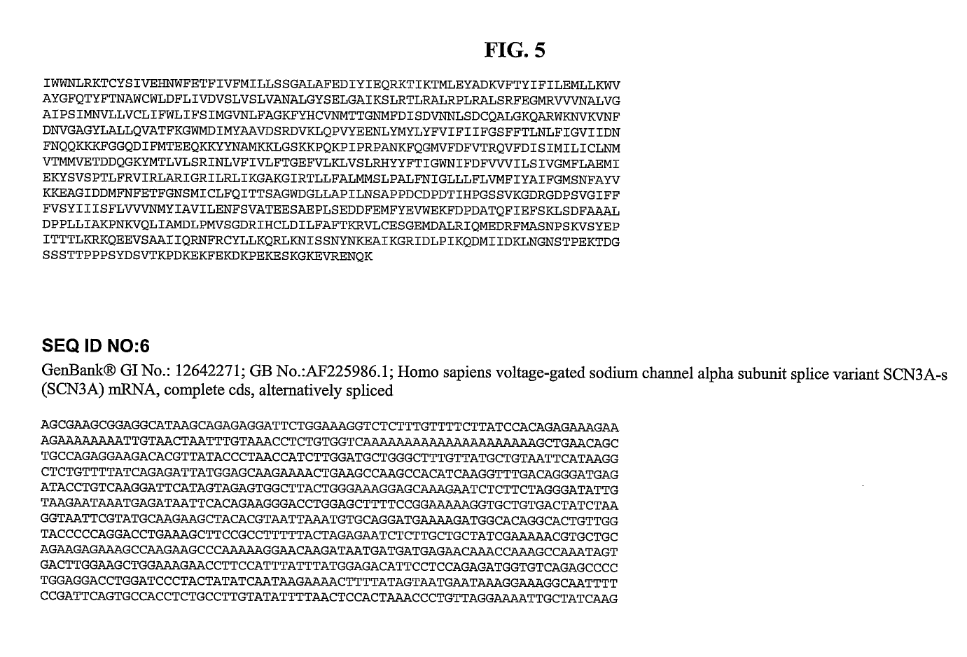

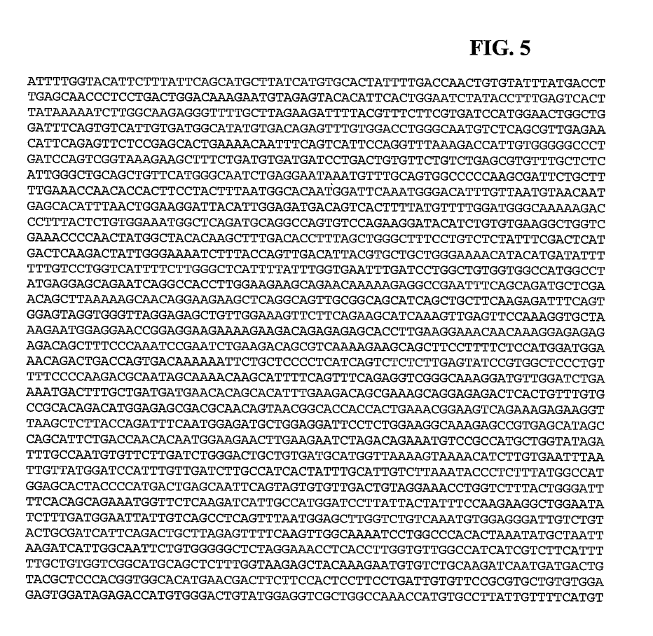

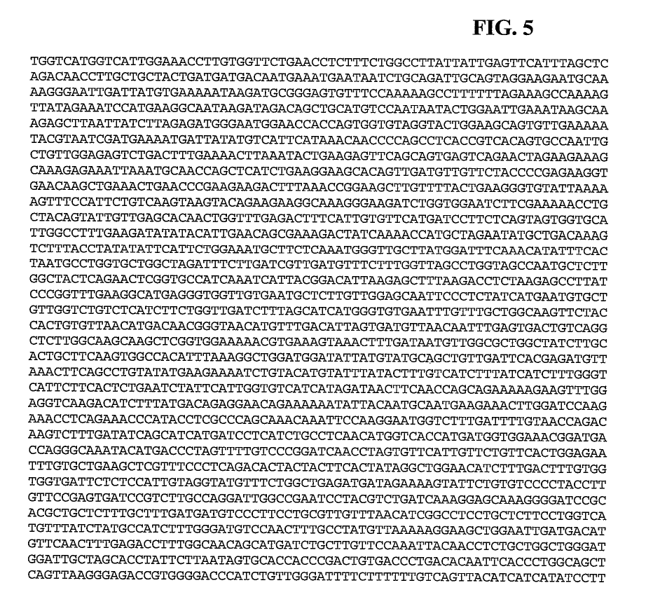

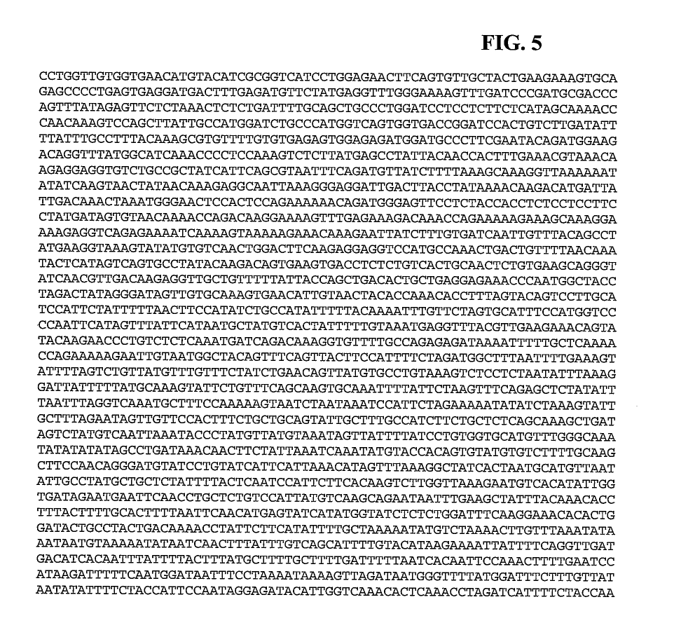

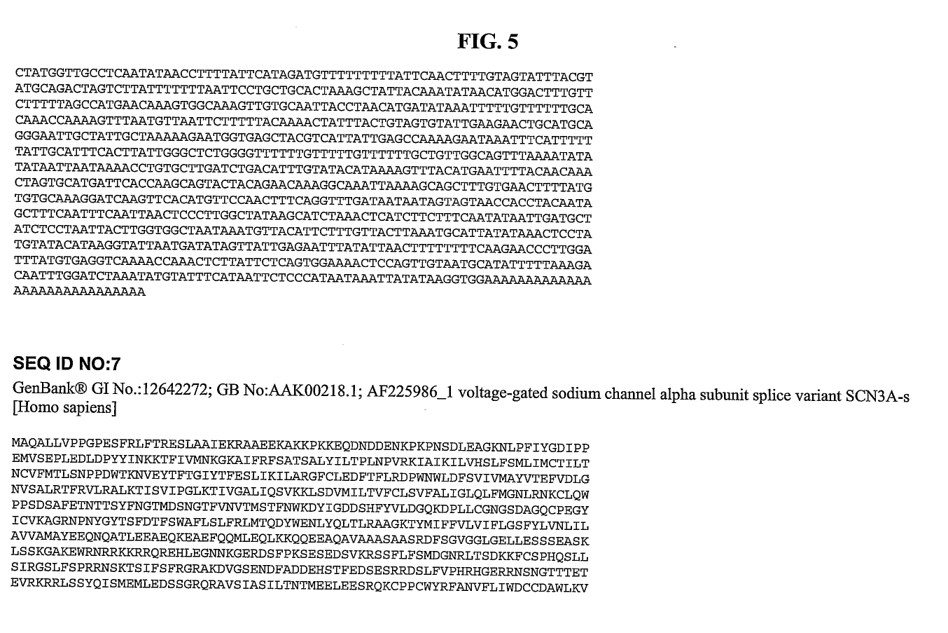

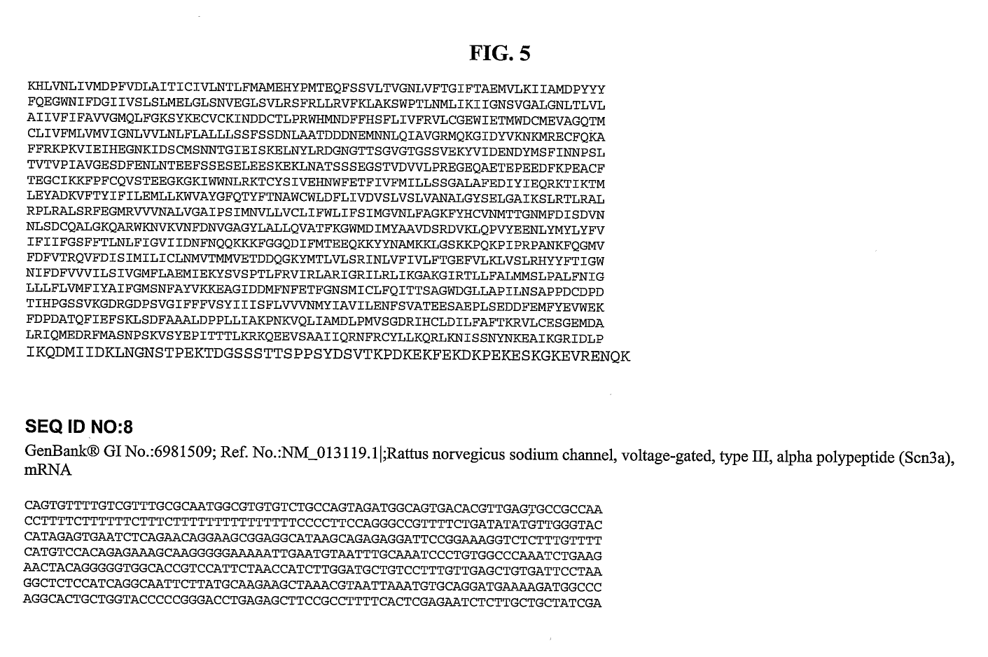

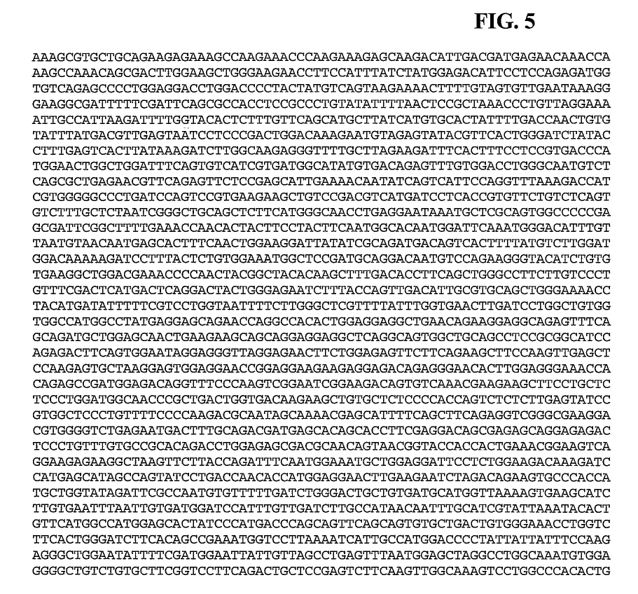

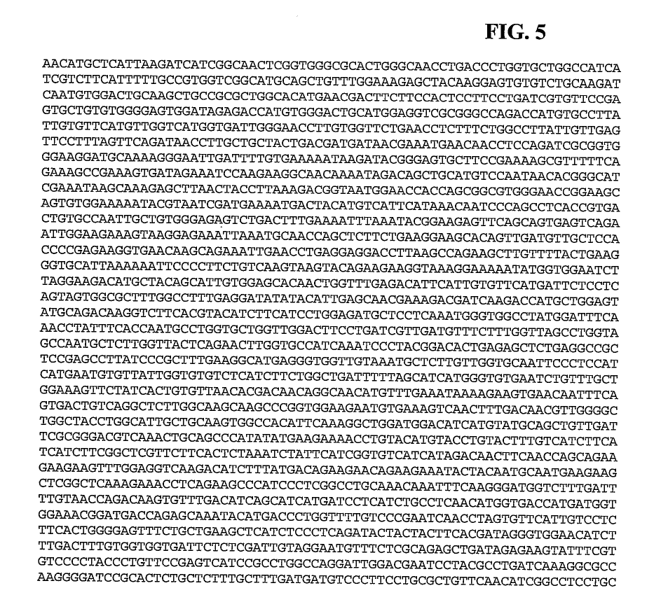

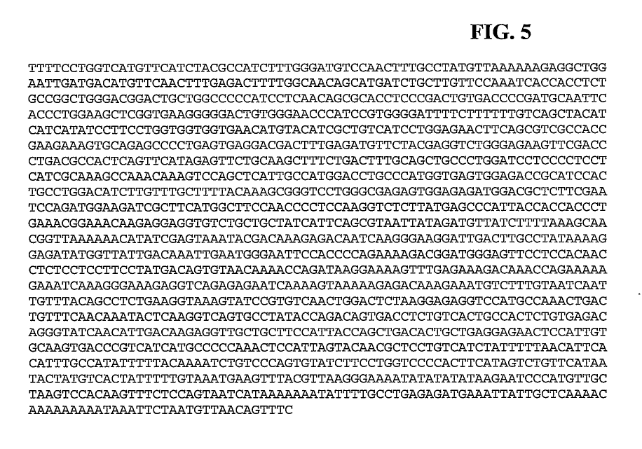

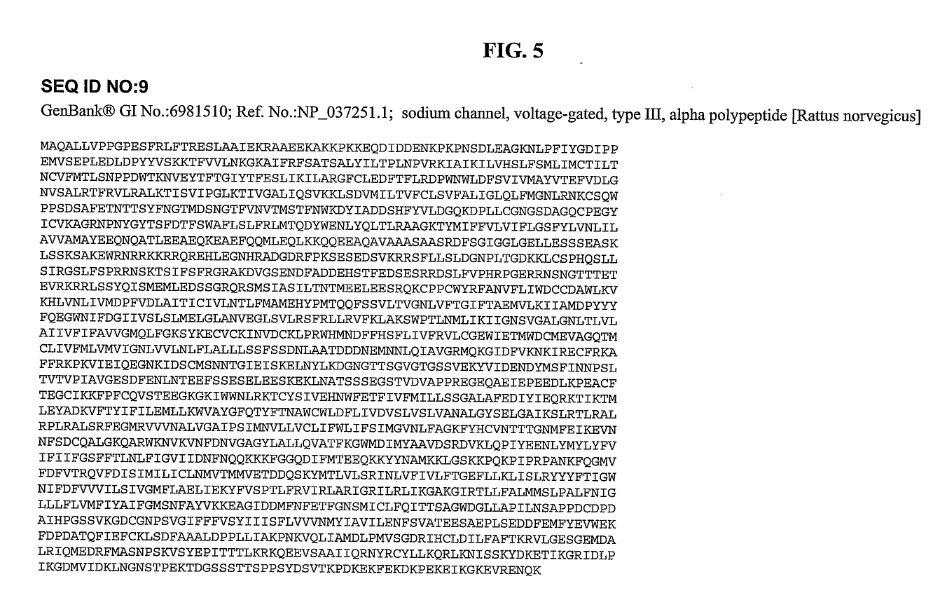

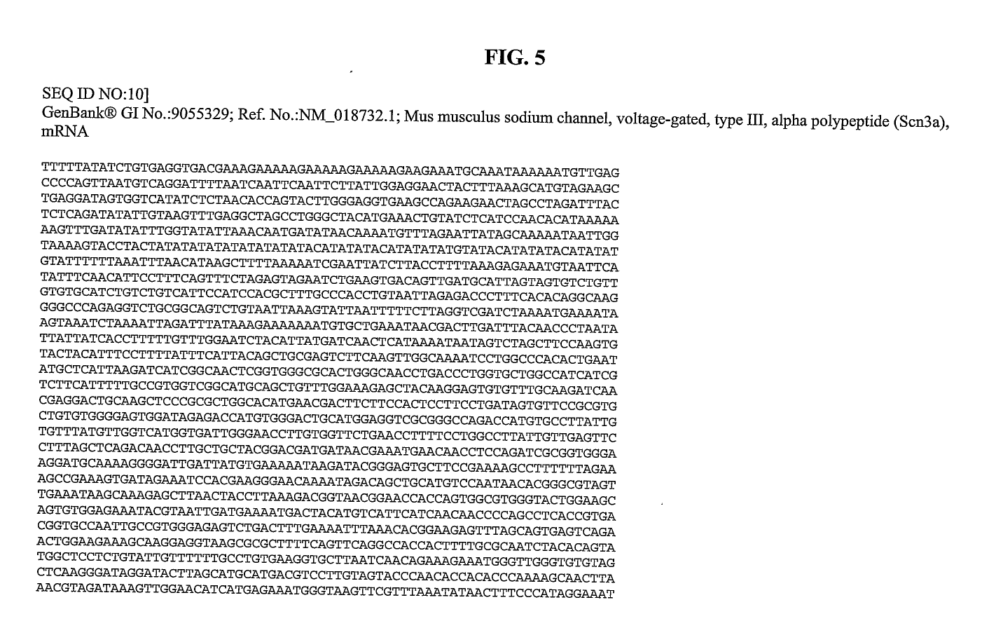

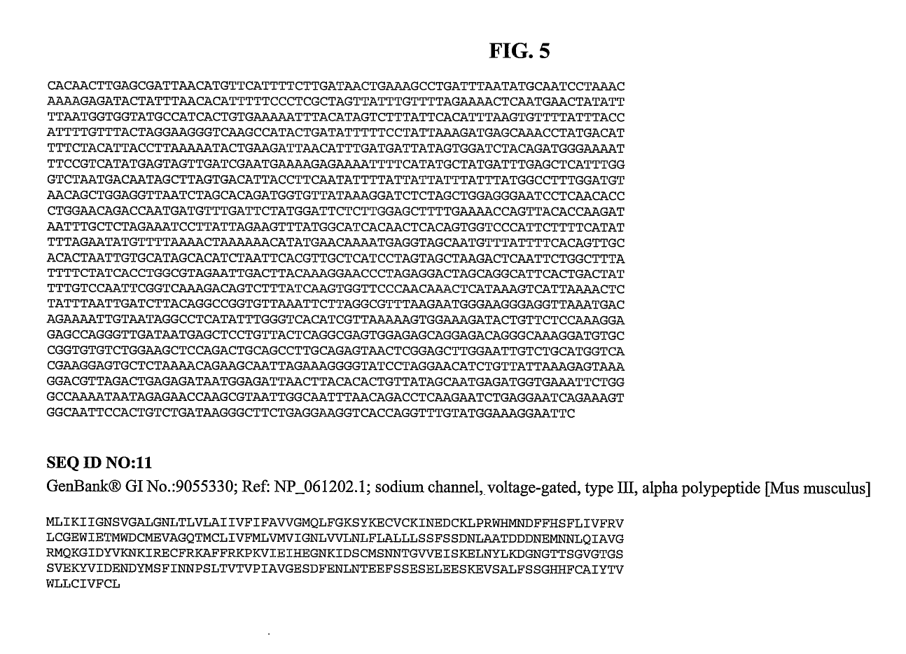

The functional properties of the mouse Naᵥ1.3 α subunit isolated in Example 1 were analyzed in the Xenopus oocyte expression system using the two electrode voltage clamp technique. The Naᵥ1.3 α subunit cDNA was amplified and subcloned into the pcDNA6 vector (Invitrogen) by standard cloning methods. Approximately forty-six nanoliters of the mouse Naᵥ1.3 α subunit (2 ng/μl) cDNA was injected into defolliculated Xenopus oocytes and currents were measured 5 days later. Sodium currents from six cells were recorded using electrodes of 0.5-1.0 MΩ resistance (3 M KCl) in a solution of 1.8 mM CaCl₂, 5 mM HEPES-Na, 2 mM KCl, 1 mM MgCl and 96 mM NaAc, pH 7.5. The oocytes were held at −100 mV and depolarized to voltages ranging from −80 mV to 50 mV. Data was acquired using a P/4 leak subtraction protocol. FIG. 3 shows representative sodium currents at different depolarizing voltages. The normalized, averaged peak current-voltage relationship of the six cells is shown in FIG. 4. The channels begin to open at −40 mV and maximum currents are reached at 0 mV. These data show that the channel encoded by the Naᵥ1.3α cDNA is functional and that it exhibits biophysical properties expected for this type of channel.

All references cited herein, whether in print, electronic, computer readable storage media or other form, are expressly incorporated by reference in their entirety, including but not limited to, abstracts, articles, journals, publications, texts, treatises, internet web sites, databases, software packages, patents, and patent publications. A number of embodiments of the invention have been described. Nevertheless, it will be understood that various modifications may be made without departing from the spirit and scope of the invention. Accordingly, other embodiments are within the scope of the following claims.

1. An isolated sodium channel type III α subunit (mNaᵥ1.3 α subunit) polypeptide, wherein the polypeptide comprises the amino acid sequence of SEQ ID NO:2.

2. The polypeptide of claim 1, wherein the polypeptide essentially consists of the amino acid sequence of SEQ ID NO:2.

3. An isolated mNaᵥ1.3 α subunit polypeptide comprising at least 10 contiguous amino acids of SEQ ID NO:2, wherein the polypeptide includes one or more of the following amino acids: isoleucine 289, proline 518, serine 728, serine 1355, asparagine 1909, threonine 1910, and valine 1921.

4. An isolated mNaᵥ1.3 α subunit nucleic acid molecule that encodes the polypeptide of claim 1.

5. The nucleic acid molecule of claim 4, wherein the nucleic acid comprises the nucleotide sequence of SEQ ID NO:1.

6. The nucleic acid molecule of claim 5, wherein the nucleic acid molecule consists essentially of the nucleotide sequence of SEQ ID NO:1.

7. (canceled)

8. A fragment of the mNaᵥ1.3 α subunit nucleic acid molecule of claim 4, wherein the fragment encodes one or more of the following amino acids: isoleucine 289, proline 518, serine 728, serine 1355, asparagine 1909, threonine 1910, and valine 1921.

9. An expression vector comprising the mNaᵥ1.3 α subunit nucleic acid molecule of claim 4 operably linked to a promoter.

10. A host cell comprising the nucleic acid of claim 4.

11. An agent which preferentially binds to the mNaᵥ1.3 α subunit polypeptide of claim 1.

12. An agent which binds selectively to the mNaᵥ1.3 α subunit polypeptide of claim 1 and not to a sodium channel type I or type II α subunit polypeptide.

13. The agent of claim 12, wherein the agent is a small molecule, a nucleic acid, or a protein.

14. (canceled)

15. (canceled)

16. (canceled)

17. A pharmaceutical composition comprising the agent of claim 12 and a pharmaceutically acceptable carrier.

18. A method for modulating a mNaᵥ1.3 α subunit polypeptide activity in a cell, the method comprising:
providing a sodium channel comprising a mNaᵥ1.3 α subunit polypeptide, wherein the mNaᵥ1.3 α subunit polypeptide is according to claim 1; and
contacting the channel with an amount of a mNaᵥ1.3 α subunit polypeptide modulator effective to modulate an activity of the mNaᵥ1.3 α subunit polypeptide.

19. (canceled)

20. A method for identifying an agent that modulates the activity of a mNaᵥ1.3 α subunit polypeptide, the method comprising:
providing a first sodium channel comprising a mNaᵥ1.3 α subunit polypeptide, wherein the mNaᵥ1.3 α subunit polypeptide is according to claim 3;
contacting the channel with a test compound; and
evaluating an activity of the sodium channel, wherein a change in activity relative to a reference value is an indication that the compound is an agent that modulates the channel.

21.-39. (canceled)

40. The method of claim 20, wherein the mNaᵥ1.3 α subunit polypeptide comprises the amino acid sequence of SEQ ID NO:2.

41. A method for identifying an agent useful in the treatment of a disorder related to sodium current modulation, the method comprising:
providing a sodium channel comprising a mNaᵥ1.3 α subunit polypeptide according to claim 3;
contacting the channel with a test compound; and
evaluating an activity of the channel, wherein a change in activity relative to a reference value is an indication that the test compound is an agent useful in a disorder related to sodium current.

42. (canceled)

43. The method of claim 41, further comprising administering the compound in vivo.

44. (canceled)

45. (canceled)

46. A method for treating a subject having a disorder related to sodium channel current, the method comprising:
identifying an agent that selectively binds a mNaᵥ1.3 α subunit polypeptide, and administering to a subject in need of such treatment a pharmacological agent which is selective for a sodium channel comprising a mNaᵥ1.3 α subunit polypeptide.

47. The method of claim 46, wherein the disorder is pain, paraesthesia, stroke, head trauma, a neurodegenerative disorder, or a disorder related to hyperexcitability of neurons.
